(19) United States
(12) Patent Application Publication
(10) Pub. No.: US 2024/0167097 A1
(43) Pub. Date: May 23, 2024

(54) CELLULAR RESPONSE ASSAYS FOR LUNG CANCER

(71) Applicants: PRECYTE INC., Seattle, WA (US); SEATTLE CHILDREN'S HOSPITAL, DBA SEATTLE CHILDREN'S RESEARCH INSTITUTE, Seattle, WA (US)

(72) Inventors: Jennifer Joy SMITH, Seattle, WA (US); Fergal Joseph DUFFY, Seattle, WA (US); Jason Douglas BERNDT, Seattle, WA (US); George Adam WHITNEY, Seattle, WA (US); Robert Jay LIPSHUTZ, Seattle, WA (US); John David AITCHISON, Seattle, WA (US); Mark David D'ASCENZO, Seattle, WA (US)

(21) Appl. No.: 18/008,043

(22) PCT Filed: Jun. 4, 2021
(86) PCT No.: PCT/US21/36000

§ 371 Date: Dec. 2, 2022

Related U.S. Application Data
(60) Provisional application No. 63/035,592, filed on Jun. 5, 2020.

(51) Int. Cl. C12Q 1/6886; C12Q 1/6809

(52) U.S. Cl. CPC C12Q 1/6886; C12Q 1/6809; C12Q 2600/118; C12Q 2600/158

(57) ABSTRACT

The present disclosure provides methods and diagnostic tools comprising classifiers and indicator cells that respond differentially to a sample derived from a subject having a disease or cancer, such as lung cancer, as compared to a control or normal sample. Also disclosed herein are methods of detecting a multicomponent gene expression readout from a genetically identical population of cells.

### CROSS-REFERENCE

This application claims the benefit of U.S. Provisional Application No. 63/035,592, filed Jun. 5, 2020, which application is incorporated herein by reference in its entirety.

### STATEMENT AS TO FEDERALLY SPONSORED RESEARCH

This invention was made with the support of the United States government under Contract numbers 1R43CA203455, 2R44CA203455, 1R44AG051282, and 2R44AG051282 by the National Institutes of Health. The government has certain rights in the invention.

### BACKGROUND

Lung cancer (both small cell and non-small cell) is the second most common cancer in both men and women. It is estimated that about 14% of all new cancers are lung cancers. Currently lung cancer is diagnosed primarily based on clinical symptoms, but for most patients, detection at this stage is often too late for effective therapy. The average 5-year survival rate is very low, but for those cases detected at an early stage (e.g., when the disease is localized), the survival rate can be increased significantly. Therefore, early cancer detection, especially detection before clinical symptoms sufficient to provide a definitive diagnosis on their own, is of critical importance.

Approaches proposed for detecting cancer in subjects have included assaying cancer cells from the cancer lesion site themselves (e.g., as opposed to assaying non-cancerous cells). The identification and use of markers from cancer cells has been suggested as a means of identifying cancer; however, a robust and accurate means for early detection of lung cancer has remained elusive. There is a need for improved methods for early detection of lung cancer in subjects and methods (e.g., non-invasive methods) of detecting lung cancer with increased diagnostic accuracy.

Low dose CT scans can be used to suggest the presence of lung cancer, either through routine screening of at-risk population, or through identification of incidental nodules in normal clinical practice. However, CT scans have greater than 90% false positive rate. Of particular concern are nodules characterized as intermediate risk, for which a full diagnosis and the development of an effective a treatment plan may be significantly more difficult. Currently available diagnostic systems result in 80% of the 5 million nodules identified each year by CT scans being characterized as intermediate risk for lung cancer, which leads to patients diagnosed with such techniques as having intermediate risk nodules being required to suffer through a diagnostic odyssey and endure invasive and dangerous follow-up tests, even though most of these patients, in fact, have benign nodules. A non-invasive diagnostic test with high sensitivity is needed to test patients with nodules of intermediate risk of malignancy and rule-out cancer for those with benign nodules.

### SUMMARY

Disclosed herein are methods and systems for detecting lung cancer and methods and systems for determining a risk for lung cancer in a subject and treatment thereof. In various aspects, a method of determining a risk for lung cancer in a subject comprises contacting an indicator cell population with a sample from the subject. In some aspects, a method of determining a risk for lung cancer in a subject comprises contacting an indicator cell population with a sample from the subject; and determining the risk for lung cancer in the subject based on a response of the indicator cell population. In some aspects, a method of determining a risk for lung cancer in a subject comprises determining the risk for lung cancer in the subject based on a response of the indicator cell population. In some aspects, the response of the indicator cell population comprises a first response pattern. In some aspects, a response pattern has one or more response pattern features. In some embodiments, a first response pattern has one or more response pattern features.

In some aspects, determining a risk for lung cancer in a subject comprises determining a first response pattern. In some aspects, the indicator cell population is a first indicator cell population. In some aspects, the subject is a first subject. In some aspects, the first subject has an unknown risk of lung cancer.

In some aspects, determining a risk for lung cancer in a subject comprises contacting a second indicator cell population with a sample from a second subject. For example, determining a risk for lung cancer in a first subject comprises contacting a second indicator cell population with a sample from a second subject, in some embodiments. In some aspects, the second subject has a known risk for lung cancer.

In some aspects, determining a risk for lung cancer in a subject comprises determining a second response pattern of a second indicator cell population. For example, determining a risk for lung cancer in a first subject comprises determining a second response pattern of a second indicator cell population, in some aspects. In some aspects, determining a risk for lung cancer in a subject comprises determining a risk for lung cancer of the first subject based on the first response pattern and the second response pattern.

In some aspects, determining a risk for lung cancer in a subject comprises determining the first response pattern, wherein the indicator cell population is a first indicator cell population and the subject is a first subject; contacting a second indicator cell population with a sample form a second subject, the second subject having a known risk for lung cancer; determining a second response pattern of the second indicator cell population; and determining a risk for lung cancer of the first subject based on the first response pattern and the second response pattern.

In some aspects, determining a risk for lung cancer in a subject comprises determining a set of key response pattern features based on the second response pattern. In some aspects, determining the risk for lung cancer of the first subject is based on the set of key response pattern features of the second response pattern and a set of key response pattern features of the first response pattern. In some aspects, the set of key response pattern features is not known before the second response pattern is determined.

In some aspects, determining a risk for lung cancer in a subject comprises determining a third response pattern of a third indicator cell population. For example, determining a risk for lung cancer in a first subject comprises determining a third response pattern of a third indicator cell population, in some embodiments. In some aspects, determining a risk for lung cancer in a subject comprises contacting the third indicator cell population with a sample from a third subject. For example, determining a risk for lung cancer in a first subject comprises contacting the third indicator cell population with a sample from the third subject, in some embodiments. In some embodiments, the third subject has a second known risk for lung cancer.

In some aspects, determining a risk for lung cancer in a subject comprises determining a response pattern for each of one or more additional indicator cell populations. For example, determining a risk for lung cancer in a first subject comprises determining a response pattern for each of one or more additional indicator cell populations, in some embodiments. In some aspects, determining a risk for lung cancer in a subject comprises contacting each of the one or more additional indicator cell populations with a sample from one or more additional subjects. In some embodiments, determining a risk for lung cancer in a first subject comprises contacting each of the one or more additional indicator cell populations with a sample from one or more additional subjects. In some embodiments, determining a risk for lung cancer in a first subject comprises contacting each of the one or more additional indicator cell populations with no more than one sample from one or more additional subjects.

In some embodiments, determining a risk for lung cancer in a subject comprises determining a differential response pattern based on two or more of the second response pattern, the third response pattern, or the response pattern for the one or more additional indicator cell populations. In some embodiments, determining a risk for lung cancer in a first subject comprises determining a differential response pattern based on two or more of the second response pattern, the third response pattern, or the response pattern for the one or more additional indicator cell populations. In some embodiments, determining a risk for lung cancer in a subject comprises determining a differential response pattern based on two or more of the second response pattern, the third response pattern, or the response pattern for each of the one or more additional indicator cell populations.

In some aspects, determining a risk of lung cancer in a subject comprises determining a set of key response pattern features based on two or more of the second response pattern, the third response pattern, or the response pattern for the one or more additional indicator cell populations. In some aspects, determining a risk of lung cancer in a first subject comprises determining a set of key response pattern features based on two or more of the second response pattern, the third response pattern, or the response pattern for the one or more additional indicator cell populations. In some aspects, determining a risk of lung cancer in a subject comprises determining a set of key response pattern features based on two or more of the second response pattern, the third response pattern, or the response pattern for each of the one or more additional indicator cell populations.

In some cases, determining a risk for lung cancer comprises measuring the set of key response pattern features of the first response pattern. In some aspects, determining the risk for lung cancer of the first subject is based on the set of key response pattern features of the first response pattern. In some aspects, determining the risk for lung cancer of the first subject is based on two or more of: the set of key response pattern features of the second response pattern, the set of key response pattern features of the third response pattern, or the set of key response pattern features of the one or more additional indicator cell populations. In some aspects, determining the risk for lung cancer of the first subject is based on: the set of key response pattern features of the first response pattern and two or more of: the set of key response pattern features of the second response pattern, the set of key response pattern features of the third response pattern, or the set of key response pattern features of the one or more additional indicator cell populations. In some aspects, determining the risk for lung cancer of the first subject is based on measured or detected properties or characteristics of an indicator cell population (e.g., measured values of detected properties or characteristics of the cells comprising the indicator cell population) comprising: the set of key response pattern features of the first response pattern and two or more of: the set of key response pattern features of the second response pattern, the set of key response pattern features of the third response pattern, or the set of key response pattern features of the one or more additional indicator cell populations.

In some aspects, the set of key response pattern features is not known before two or more of the second response pattern, the third response pattern, and the response pattern for each of the one or more additional indicator cell populations is determined.

In some aspects, the second subject is known to have lung cancer. In some aspects, the second subject is known to not have lung cancer. In some aspects, the third subject is known to have lung cancer. In some aspects, the third subject is known to not have lung cancer. In some aspects, each subject of the one or more additional subjects has a known risk for lung cancer. In some aspects, each subject of the one or more additional subjects is known to have lung cancer. In some aspects, at least one subject of the one or more additional subjects is known to not have lung cancer.

In some aspects, the set of key response pattern features is determined using a classifier. In some aspects, the set of key response pattern features is determined using a machine learning approach. In some aspects, the set of key response pattern features is determined using a supervised machine learning approach. In some aspects, the set of key response pattern features is determined using a random forest classifier. In some aspects, the set of key response pattern features is determined using a classifier, a supervised machine learning approach, or a random forest classifier. In some aspects, the set of key response pattern features is determined using an unsupervised machine learning approach. In some aspects, determining the risk for lung cancer of a subject comprises training a classifier using two or more of the second response pattern, the third response pattern, or the response pattern for each of the one or more additional indicator cell populations. In some aspects, determining the risk for lung cancer of a subject comprises training the classifier using cross-validation and a hold-out set. In some aspects, determining the risk for lung cancer of a subject comprises testing the classifier using cross-validation and a hold-out set. In some aspects, determining the risk for lung cancer of a subject comprises training or testing the classifier using cross-validation and a hold-out set. In some aspects, determining the risk for lung cancer comprises measuring one or more response pattern feature values. For example, determining the risk of lung cancer comprises measuring one or more response pattern feature values of the first response pattern, in some embodiments. For example, determining the risk of lung cancer comprises measuring one or more response pattern feature values of the second response pattern, in some embodiments. For example, determining the risk of lung cancer comprises measuring one or more response pattern feature values of the third response pattern, in some embodiments. For example, determining the risk of lung cancer comprises measuring one or more response pattern feature values of the one or more additional response patterns, in some embodiments.

In some aspects, the one or more response pattern feature values comprises one or more of: an epigenetic pattern, a gene expression level, an RNA abundance level, an intracellular protein concentration, a concentration of a low molecular weight metabolite, or a concentration of a secreted protein or cell surface protein.

In some aspects, determining the risk for lung cancer of a subject comprises measuring response pattern feature values for each response pattern feature of the set of key response pattern features in one or more of: the first population of indicator cells, the second population of indicator cells, the third population of indicator cells, or the one or more additional indicator cell populations.

In some aspects, determining the risk for lung cancer of a subject comprises measuring the one or more response pattern feature values using RNA-seq, reporter gene assay, polymerase chain reaction (PCR), enzyme-linked immunosorbent assay (ELISA), next-generation sequencing, direct nucleic acid detection with molecular barcodes, microarray analysis, analysis of cell morphology, fluorescence microscopy, cell viability, or any combination thereof. In some aspect, the sample of the first subject is a biological fluid. In some aspects, the biological fluid is blood serum or blood plasma. In some aspects, the one or more response pattern feature values comprise an expression level of a gene selected from: EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA. V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318, or any combination thereof. In some aspects, the one or more response pattern feature values comprise an expression level of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more than 35 of the genes selected from: EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA. V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some aspects, the one or more response pattern feature values comprise an expression level of at least 20 genes selected from: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some aspects, the one or more response pattern feature values comprise an expression level of each of the following genes: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318, ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2.

In some aspects, the one or more response pattern feature values comprise an expression level of at least 2, 3, 4, 5, 6, 7, 8, 9, 10, 11, 12, 13, 14, 15, 16, 17, 18, or 19 of ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2. In some aspects, the one or more response pattern feature values comprise an increase in expression level of at least 1, 2, 3, 4, 5, 6, 7, 8, 9, 10, 11, 12, 13, 14, 15, 16, 17, 18, or 19 of ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2. In some aspects, the one or more response pattern feature values comprise a decrease in expression level of at least 1, 2, 3, 4, 5, 6, 7, 8, 9, 10, 11, 12, 13, 14, 15, 16, 17, 18, or 19 of ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2. In some aspects, the one or more response pattern feature values comprise a lack of change in expression level of at least 1, 2, 3, 4, 5, 6, 7, 8, 9, 10, 11, 12, 13, 14, 15, 16, 17, 18, or 19 of ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2.

In some aspects, a method of determining a risk for lung cancer comprises measuring an expression level of a transcription factor in an indicator cell population. In some cases, the transcription factor is HIF1-alpha. In some aspects, determining a risk for lung cancer is determined based on the measured expression level of the transcription factor. In some cases, the expression level of the transcription factor is measured to be increased. In some cases, the transcription factor is HIF1-alpha. In some cases, the expression level of HIF1-alpha is measured to be increased.

In some aspects, the risk of lung cancer in the subject is determined based on data from a CT scan. In some aspects, the risk of lung cancer in the subject is determined based at least in part on data from a CT scan. In some aspects, the risk of lung cancer in the subject is determined based on data from a CT scan and one or more response pattern feature values measured (or detected) in an indicator cell population (e.g., after contacting the indicator cell population with a sample). In some aspects, the risk of lung cancer in the subject is determined based on data from a CT scan and one or more gene expression levels measured (or detected) in an indicator cell population (e.g., after contacting the indicator cell population with a sample). In some aspects, the risk of lung cancer in the subject is determined based on data from a CT scan of the patient and one or more additional aspects (e.g., clinically assessed aspects) of the patient's condition. In some aspects, the first indicator cell population comprises a clonal cell population derived from stem cells. In some aspects, the second indicator cell population comprises a clonal cell population derived from stem cells. In some aspects, the first indicator cell population comprises an alveolar cell, a lung epithelial cell, an immune cell, an endothelial cell, a fibroblast, or a combination thereof. In some aspects, the second indicator cell population comprises an alveolar cell, a lung epithelial cell, an immune cell, an endothelial cell, a fibroblast, or a combination thereof. In some aspects, the third indicator cell population comprises an alveolar cell, a lung epithelial cell, an immune cell, an endothelial cell, a fibroblast, or a combination thereof. In some aspects, the one or more additional indicator cell population comprises an alveolar cell, a lung epithelial cell, an immune cell, an endothelial cell, a fibroblast, or a combination thereof.

In some aspects, determining a risk for lung cancer of the first subject comprises determining that the first subject has lung cancer. In some aspects, determining a risk for lung cancer of the first subject comprises determining that the first subject does not have lung cancer. In some aspects, the lung cancer is selected from the group: non-small cell lung cancer, adenocarcinoma, squamous cell carcinoma, or large cell carcinoma. In some aspects, the lung cancer is pre-symptomatic or pre-invasive. In some aspects, the first subject has an indeterminate pulmonary nodule (IPN). In some aspects, the IPN is 3-25 mm or less than 30 mm. In some aspects, the first subject has a nodule or IPN with an intermediate risk for lung cancer. In some aspects, the first subject's risk for lung cancer is from 5 percent to 65 percent. In some aspects, determining a risk for lung cancer comprises determining that the IPN is a benign nodule. In some aspects, determining a risk for lung cancer comprises determining that the IPN is a non-benign nodule. In some aspects, determining risk of lung cancer comprises determining the percentage risk. In some aspects, percentage risk is calculated using pretest probability and likelihood ratio from the classifier using Fagan's nomogram or another tool.

In some aspects, the method has an accuracy rate of at least 70% in detecting lung cancer. In some aspects, the method has a sensitivity of at least 95% and a specificity of at least 45%. In some aspects, the method has a negative predictive value of at least 90%.

In some aspects, a method disclosed herein comprises determining a treatment for the first subject based on the determined risk for lung cancer of the first subject. In some aspects, a method disclosed herein comprises administering the treatment to the first subject. In some aspects, the treatment comprises gene therapy, small molecule therapy, treatment with a small molecule, chemotherapy, immunotherapy, surgery, radiosurgery, proton therapy, radiation therapy, photodynamic therapy, targeted therapy, or any combination thereof. In some aspects, chemotherapy comprises treatment with ethothrexate, everolimus, alectinib, pemetrexed disodium, brigatinib, atezolizumab, bevacizumab, carboplatin, ceritinib, crizotinib, ramucirumab, dabrafenib, docetaxel, erlotinib hydrochloride, methotrexate, afatinib dimaleate, gemcitabine hydrochloride, gemcitabine hydrochloride, gefitinib, trametinib, methotrexate, mechlorethamine hydrochloride, vinorelbine tartrate, necitumumab, nivolumab, osimertinib, paclitaxel, carboplatin, pembrolizumab, pemetrexed disodium, necitumumab, ramucirumab, dabrafenib, osimertinib, erlotinib hydrochloride, paclitaxel, docetaxel, atezolizumab, trametinib, vinorelbine tartrate, crizotinib, ceritinib, carboplatin-taxol, gemcitabine-cisplatin, doxorubicin hydrochloride, etoposide, topotecan hydrochloride, mechlorethamine hydrochloride, topotecan hydrochloride, or any combination thereof.

In some aspects, the subject is a human. In some aspects, the subject is a non-human.

In various aspects, a system for determining a risk of lung cancer in a first subject comprises a first indicator cell population. In various aspects, a system for detecting lung cancer in a first subject comprises a sample from the first subject. In various aspects, a system for detecting lung cancer in a first subject comprises an imaging module configured to detect a first signal from the first indicator cell population. In various aspects, a system for detecting lung cancer in a first subject comprises a computer in communication with the detector, comprising a processor and a non-transitory memory on which is stored instructions that, when executed, cause the processor to: determine the risk for lung cancer in the first subject based on the first signal using a classifier stored in the non-transitory memory of the computer. In various aspects, a system for detecting lung cancer in a first subject comprises a first indicator cell population; a sample from the first subject; an imaging module configured to detect a first signal from the first indicator cell population; and a computer in communication with the detector, comprising a processor and a non-transitory memory on which is stored instructions that, when executed, cause the processor to: determine the risk for lung cancer in the first subject based on the first signal using a classifier stored in the non-transitory memory of the computer. In some aspects, a system for detecting lung cancer in a first subject comprises a second indicator cell population; and a sample from a second subject having a known risk for lung cancer, wherein the imagine module is configured to detect a second signal from the second indicator cell population; and wherein the instructions, when executed further cause the processor to: determine a first response pattern based on the first signal, determine a second response pattern based on the second signal, and determine a risk for lung cancer of the first subject based on the first response pattern and the second response pattern using the classifier. In some aspects, the instructions, when executed cause the processor to determine a set of key response pattern features based on the second response pattern. In some aspects, the instructions, when executed, cause the processor to determine a set of key response pattern feature values of the first response pattern based on the set of key response pattern features and a set of response pattern feature values of the first response pattern. In some aspects, determining a risk for lung cancer in a first subject is based on the set of key response pattern feature values of the first response pattern.

In some aspects, determining the first response pattern comprises operating the imaging module to detect the first signal after the first indicator cell population is contacted with the sample from the first subject. In some aspects, determining the second response pattern comprises operating the imaging module to detect the second signal after the second indicator cell population is contacted with the sample from the second subject. In some aspects, an iCAP system or method described herein (e.g., comprising operating an imaging module) comprises detecting (or measuring) one or more parameters of one or more indicator cell populations (e.g., morphological parameters, such as cell circumference, cell area, cell volume, nucleus area, nucleus volume, nucleus location, cell membrane smoothness, nucleus roundness, cell viability, cell membrane texture, protein subcellular distribution and/or localization, cell heterogeneity, organelle structural changes cell-to-cell proximity, or cell-to-cell contact, and/or non-morphological parameters, such as cell metabolic activity, cell proliferation, biological activity, cell subpopulation redistribution, cell redox state, cell membrane potential, the presence, absence, or abundance of cell differentiation markers, cell migration, cell cycle regulation indicators such as expression level of cell cycle checkpoint proteins, molecular uptake kinetics, cell surface receptor activity, enzyme activation, protein modification, protein expression, protein translation, cell secretion, fluorescent or nonfluorescent imaging particle detection) and/or changes (increases, decreases, for example, relative to an indicator cell population uncontacted with the sample, e.g., the same population prior to contact with the sample) and/or a lack of change thereof.

In some aspects, the set of key response pattern features is not known before the second response pattern is determined.

In some aspects, the instructions, when executed, cause the processor to determine a third response pattern of a third indicator cell population after the third indicator cell population is contacted by a sample from a third subject.

In some aspects, the instructions, when executed, cause the processor to determine a response pattern for each of one or more additional indicator cell populations after the one or more additional indicator cell populations are contacted by a sample of one or more respective subjects. In some aspects, the instructions, when executed, cause the processor to determine a response pattern for each of one or more additional indicator cell populations after the one or more additional indicator cell populations are contacted by a sample of nor more than one additional subject.

In some aspects, the instructions, when executed, cause the processor to determine a differential response pattern based on two or more of the second response pattern, the third response pattern, or the response pattern for each of the one or more additional indicator cell populations. In some aspects, the instructions, when executed, cause the processor to determine a set of key response pattern features based on two or more of the second response pattern, the third response pattern, or the response pattern for each of the one or more additional indicator cell populations.

In some aspects, determining the risk for lung cancer of the first subject is based on: the set of key response pattern feature values of the first response pattern; and two or more of: a set of key response pattern feature values of the second response pattern; a set of key response pattern feature values of the third response pattern; and a set of key response pattern feature values of the one or more additional indicator cell populations.

In some aspects, the second subject is known to have lung cancer. In some aspects, the second subject is known to not have lung cancer. In some aspects, the third subject is known to have lung cancer. In some aspects, the third subject is known to not have lung cancer. In some aspects, each subject of the one or more additional subjects has a known risk for lung cancer. In some aspects, each subject of the one or more additional subjects is known to have lung cancer. In some aspects, at least one subject of the one or more additional subjects is known to not have lung cancer.

In some aspects, the set of key response pattern features is determined using a classifier. In some aspects, the set of key response pattern feature is determined using a machine learning approach. In some aspects, the set of key response pattern features is determined using a supervised machine learning approach. In some aspects, the set of key response pattern features is determined using a random forest classifier. In some aspects, the set of key response pattern features is determined using a classifier, a supervised machine learning approach, or a random forest classifier. In some aspects, the set of key response pattern features is determined using an unsupervised machine learning approach. In some aspects, the instructions, when executed, cause the processor to train the classifier using two or more of the second response pattern, the third response pattern, or the response pattern for each of the one or more additional indicator cell populations.

In some aspects, one or more response pattern feature values of the set of key response pattern features comprises one or more of: an epigenetic pattern, a gene expression level, an RNA abundance level, an intracellular protein concentration, a concentration of a low molecular weight metabolite, or a concentration of a secreted protein or cell surface protein. In some aspects, operating the imaging module comprises performing an RNA-seq assay, a reporter gene assay, a polymerase chain reaction (PCR) assay, an enzyme-linked immunosorbent assay (ELISA), next-generation sequencing, direct nucleic acid detection with molecular barcodes, microarray analysis, analysis of cell morphology, fluorescence microscopy, cell viability, or any combination thereof.

In some aspects, the sample of the first subject is a biological fluid. In some aspects, the biological fluid is blood serum or blood plasma.

In some aspects, the one or more response pattern feature values comprise an expression level of a gene selected from: EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318 or any combination thereof. In some aspects, the one or more response pattern feature values comprise an expression level of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more than 35 of the genes selected from: EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some aspects, the accuracy of an iCAP system can be improved when the one or more response pattern feature values used in an iCAP system comprise an expression level of at least 20 genes selected from: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318, ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2. In some aspects, the accuracy of an iCAP system can be improved when the one or more response pattern feature values used in an iCAP system comprise an expression level of each of the following genes: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some aspects, the accuracy of an iCAP system can be improved when the one or more response pattern feature values used in an iCAP system comprise an expression level of each of the following genes: CACNG6, PRKCA, ROR2, RSBN1, PDZD7, CCDC66, ANKRD37, HAGHL, MT-ND4, BMP6, RASAL1, CEMIP, SPOCD1, PRR22, IFNL2, TRIM2, KIRREL2, CTF1, ARMCX4, and IFNK. In some aspects, the accuracy of an iCAP system can be improved when the one or more response pattern feature values used in an iCAP system comprise an expression level of each of the following genes: CACNG6, PRKCA, ROR2, RSBN1, PDZD7, CCDC66, ANKRD37, HAGHL, MT-ND4, BMP6, RASAL1, CEMIP, SPOCD1, PRR22, IFNL2, TRIM2, KIRREL2, CTF1, ARMCX4, and IFNK.

In some aspects, the risk of lung cancer in the subject is determined based on an expression level of a transcription factor measured in an indicator cell population. In some aspects, the transcription factor is HIF1-alpha. In some aspects, the risk of lung cancer in the subject is determined based on data from a CT scan. In some aspects, the risk of lung cancer in the subject is determined based on data from a CT scan of the patient or on data from a CT scan and one or more additional aspects (e.g., clinically assessed aspects) of the patient's condition.

In some aspects, the first indicator cell population comprises a clonal cell population derived from stem cells. In some aspects, the second indicator cell population comprises a clonal cell population derived from stem cells. In some aspects, the first indicator cell population comprises an alveolar cell, a lung epithelial cell, an immune cell, an endothelial cell, a fibroblast, or a combination thereof. In some aspects, the second indicator cell population comprises an alveolar cell, a lung epithelial cell, an immune cell, an endothelial cell, a fibroblast, or a combination thereof.

In some aspects, determining a risk for lung cancer of the first subject comprises determining that the first subject has lung cancer. In some aspects, determining a risk for lung cancer of the first subject comprises determining that the first subject does not have lung cancer. In some aspects, the lung cancer is selected from the group: non-small cell lung cancer, adenocarcinoma, squamous cell carcinoma, or large cell carcinoma. In some aspects, the lung cancer is pre-symptomatic or pre-invasive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a schematic of an indicator cell assay platform (iCAP), according to some embodiments. Shades of gray in the cellular response output reflect levels of gene expression.

FIG. 2A-FIG. 2C illustrate diagrams of methods of determining a differential response pattern, according to some embodiments.

FIG. 3A-FIG. 3C show receiver operating characteristic (ROC) curves illustrating independent validation of iCAP classifiers, and principal component analysis (PCA) showing sequencing batch effect, according to some embodiments. FIG. 3A shows validation of iCAP classifiers (with 25 differentially expressed genes (DEGs) as features+nodule size (solid black line) or 100 differentially expressed genes (DEGs) as features+nodule size (solid gray line)) with a holdout set of 73 independent samples (confidence interval shown in parentheses), according to some embodiments. FIG. 3B shows validation of 25 DEG+nodule size classifier with both hold out sets of 103 (73+30) samples (confidence interval shown in parentheses), according to some embodiments. FIG. 3C shows principal component analysis (PCA) illustrating sample cluster by RNAseq library prep batch, indicating high technical variability in the data due to sequencing batch effect, according to some embodiments. Each point indicates iCAP data for an individual sample for these representative examples of collected data and the diagonal line separates samples processed in two different RNAseq library preparation batches.

FIG. 4A shows a comparison of gene expression in two indicator cell types used in cellular response assays, according to some embodiments. FIG. 4B shows a log 2 fold change comparison of differential gene expression in two indicator cell types, according to some embodiments. Count is used as a measure of transcript abundance, in these representative examples of collected data.

FIG. 5 illustrates results from an example of a factorial experiment used to evaluate plasma concentration and incubation time in an indicator cell assay according to some embodiments disclosed herein, with the RNA yield (ng) plotted across various iCAP conditions. Shades of gray reflect RNA yield with higher yields indicated by lighter intensity.

FIG. 6A-FIG. 6C illustrate box and whisker plots of the average expression of a gene of interest (e.g., WASH7P) across three iCAP batches using three separate normalization methods.

FIG. 7 illustrates receiver operator characteristic (ROC) curves showing performances of three lung cancer classifiers (dashed line indicates nodule size classifier; gray line indicates iCAP classifier; thick black line indicates iCAP+nodule size classifier), according to some embodiments. Samples in training and test sets were processed in the same RNAseq library prep batch for the production of these data.

FIG. 8A and FIG. 8B illustrate detection of differential expression for 58 genes in the iCAP between treatment with samples from patients with malignant lung cancer and benign nodules (n=6) using direct nucleic acid detection with molecular barcodes technology (nCounter technology, Nanostring) (FDR<0.1), in accordance with some embodiments. In each plot, data presented on the left and right are from benign and malignant samples, respectively.

FIGS. 9A-9C. illustrate gene expression levels of iCAP biomarkers from samples from patients with benign and malignant nodules (FDR<0.02), in accordance with some embodiments. Data from benign and malignant samples are presented on the left and right sides, respectively, of each of FIG. 9A, FIG. 9B, and FIG. 9C.

FIG. 10. illustrates unsupervised hierarchical clustering of iCAP gene expression data, in accordance with some embodiments.

FIG. 11. illustrates performance of iCAP model M6 with and without inclusion of patient clinical data as a feature in the model. ROC curves are shown for three different models: 1) M6, which uses only iCAP gene-expression data as features, 2) Special Pulmonary Nodule (SPN) malignancy score (“SPN Clinical Malignancy Score”), which is based solely on the SPN malignancy risk score for each patient (derived solely from clinical data from each patient), and 3) Modified M6 model, which comprises features from M6 (which can utilize an iCAP gene expression feature system) and a single-feature malignancy risk score.

### DETAILED DESCRIPTION

The present disclosure provides systems, compositions, and methods for the detection or early diagnosis of a physiological condition or disease, such as lung cancer. As disclosed herein, one or more biological sample from a subject can be assayed to produce a data set (e.g., a response pattern comprising response pattern feature values) indicative of one or more physiological conditions of the subject, such as the presence or absence of lung cancer or the presence or absence of a specific type of lung cancer, or a certain risk of a subject having lung cancer. In many cases, a response pattern, comprising one or more measured or determined parameters (e.g., one or more response pattern features), is used to determine the presence, absence, risk, or type of lung cancer in a subject. In many cases, a response pattern feature is assayed using a population of indicator cells, e.g., by experimentally detecting, measuring, or determining a value of a parameter for all or a portion of the indicator cell population according to the methods and systems described herein. For example, a sample from a subject can be brought into contact with an indicator cell population (e.g., one or more indicator cells), which can result in a change to the value of one or more parameters of the indicator cell(s) of the indicator cell population. The value measured or determined for one or more parameter(s) of an indicator cell population, which can include, e.g., a level or change in level of the production of a protein, the expression of one or more genes, or the epigenetic state of a nucleic acid in the indicator cell, can be a response pattern feature value in the methods and systems disclosed herein. As described herein, determining the values of a response pattern (e.g., response pattern feature values) and determining a specific set of features comprising a response pattern can be used to determine the presence of, the risk of, or the progression of a physiological condition, such as lung cancer, in a subject. Assaying indicator cells in vitro (e.g., as compared to assaying cells in situ) can be critical to obtaining a strong, clean signal from the assayed cells, for example, wherein the signal is in response to the applied sample (e.g., and not influenced by local or systemic input from the biological system) and is not affected (e.g., altered or decreased) during isolation of the assayed cells. In some cases, an indicator cell assay platform (iCAP) system or method can have high sensitivity (95% or greater), which can be important or in some cases necessary for minimizing false negative rate, which in turn can be important for avoiding the misclassification of malignant tumors as non-malignant or benign tumors.

Despite the potential for high variability in the factors present in a given biological sample, the identities and/or quantities of factors present in the sample (e.g., factors used to evaluate the sample or the physiological state of a subject from which the sample was derived, which can include biomarkers in the sample) do not need to be known prior to the use of the systems, compositions, and methods disclosed in determining a physiological state or determining that a sample is derived from a patient with a disease (such as lung cancer). In some cases, a determination of a physiological state (e.g., the presence or absence of lung cancer or a specific type or stage thereof, or a risk for lung cancer or a specific type or stage thereof) can be made and/or a distinction can be drawn between closely related physiological states even when information about a sample or indicator response pattern used in the systems, compositions, and methods disclosed herein is incomplete. For instance, a determination of whether a sample is from a patient with a specific type of lung cancer (e.g., adenocarcinoma, squamous cell lung cancer, or large cell lung cancer) can be made using the systems, methods, or compositions disclosed herein, even if the number or identity of one or more factors present in a sample that are used to make such a determination is/are not known beforehand. Further, a determination of whether a sample is from a patient with a specific condition (e.g., adenocarcinoma, squamous cell lung cancer, or large cell lung cancer) can be made using the systems, methods, or compositions disclosed herein, even if the identity of one or more features of an indicator cell response pattern used in making such a determination is/are not known beforehand.

Indicator Cell Assay Platforms (iCAPs)

An indicator cell assay platform (iCAP), or a system, kit, or method of use thereof, can be used to detect or determine a risk for lung cancer or to differentiate among different types of lung cancer in a test subject (e.g., a human or preclinical animal model). An iCAP can comprise a cellular component (e.g., one or more population of indicator cells). When contacted by a sample derived from a subject (e.g., a cellular sample or a non-cellular sample, such as a blood serum sample), an indicator cell (or population of indicator cells) can produce one or more detectable or measurable signals or characteristics (e.g., expression level or change in an expression level of a gene of an indicator cell) that are informative about one or more physiological states of the sample and/or the subject from which the sample was derived. In some cases, a signal or characteristic or change in a signal or characteristic (e.g., as detected or measured according methods disclosed herein) of an indicator cell or an indicator cell population (e.g., in response to the indicator cell(s) being contacted by a sample) comprises a feature of a response pattern of the indicator cell or indicator cell population of an iCAP system or method. In some cases, each measured or detected signal or characteristic of an indicator cell or indicator cell population (e.g., that results from the indicator cell or an indicator cell population being contacted by a sample) comprises a feature of a response pattern of the indicator cell or indicator cell population.

The response pattern produced by an indicator cell of an iCAP can comprise a value of response pattern feature (e.g., a value of a parameter measured or determined, as disclosed herein). In some cases, the response pattern produced by an indicator cell of an iCAP comprises a plurality of response pattern features. A feature of a response pattern can comprise an individual, measurable property or characteristic (e.g., a measured or detected property or characteristic) of one or more cells of the indicator cell population or a change in a characteristic of one or more cells of the indicator cell population. For example, a response pattern feature can comprise a value (e.g., a measured or detected value) or a change in a value of one or more parameter of the indicator cell (e.g., one or more properties or characteristics of the indicator cell, such as a biomarker), such as the abundance level of a specific RNA molecule or protein. For example, an expression level or change in an expression level of a gene of an indicator cell (e.g., after being contacted by a sample) can be a feature of a response pattern of an indicator cell of an iCAP. In some such cases, a response pattern feature value can be the quantitative or qualitative value measured or detected for the response pattern feature parameter obtained during a specific experiment or plurality of experiments. In some cases, a response pattern feature value (e.g., a change in an expression level of a gene) can be an increase (e.g., an increase in the expression level of the gene, for example, of an indicator cell population after contacting the cell population with a sample). In some cases, a response pattern feature value (e.g., a measured change in an expression level of a gene) can be a decrease (e.g., a decrease in the expression level of the gene, for example, of an indicator cell population after contacting the cell population with a sample). In some cases, a response pattern feature value can be a lack of change in the expression level of a gene (e.g., no change in the expression level of a gene, for example, of an indicator cell population after contacting the cell population with a sample). In some cases, a cell parameter (e.g., a biomarker) of a system, composition, or method disclosed herein is not within or attached to a cell (e.g., a secreted protein or a protein or nucleic acid of a lysed cell). Data comprising a response pattern can be analyzed (e.g., using a system or method comprising the creation and/or use of one or more classifier) to determine a physiological state (e.g., the presence or absence of a lung cancer or a risk thereof) in a subject or sample. For example, determining a risk for lung cancer of a subject can be based on a set of measured response pattern features (e.g., key response pattern features, as described herein). In some cases, determining a risk for lung cancer in a subject is based on a comparison of a set of response pattern features (or, in some cases, response pattern feature values) of a first population of indicator cells contacted with a sample from a first subject with an analogous set of response pattern features (or, in some cases, response pattern feature values) of a second population of indicator cells contacted with a sample from a second subject.

Aspects of an iCAP system, composition, or method (e.g., one or more sets of response pattern features and/or one or more sets of key response pattern features, as described herein) can be selected specifically for detection and/or evaluation of a specific physiological state (e.g., the presence of lung cancer or an increased or heightened risk of lung cancer) or a class of physiological states. An iCAP system, compositions, or method can be optimized for detection and/or evaluation of a specific physiological state or a class of physiological states through the use of specific elements, components, or steps, as disclosed herein. In some cases, an iCAP system or method is improved or optimized by determining a set of response pattern features and/or key response pattern features for a detection or determination of a specific physiological state or set of physiological states in a subject (e.g., wherein the subject's sample is used in the iCAP system). In some cases, determining a set of response pattern features and/or key response pattern features comprises selecting a set of response pattern features and/or key response pattern features from one or more larger sets of possible response pattern features and/or key response pattern features, e.g., as described herein.

In some cases, one or more cells of a certain cell type or specific cell population can be selected for use as an indicator cell in an iCAP for the detection or evaluation of a physiological state for reasons that include empirical data supporting the cells' utility in such an iCAP system or method. For example, an epithelial cell population may be used in an iCAP system, kit, or method (e.g., as an indicator cell population) for the detection or evaluation of lung cancer (e.g., a lung cancer iCAP) because of the cell type's ability to produce a response pattern (e.g., when contacted by a sample) that is useful in distinguishing between the presence or absence of lung cancer when compared to a second response pattern (e.g., in the training of a lung cancer classifier or in the evaluation of a test sample derived from a subject). In some cases, it can be advantageous to select an indicator cell derived from a subject with similar biographical or medical background information (e.g., race, gender, age, risk history, or clinical presentation) as a test subject (or a plurality of test subjects) for use in an iCAP system to assay the test subject (or test subjects') sample(s). In some cases, indicator cells derived from or immortalized from a cell derived from a subject of similar biographical or medical background can improve the accuracy and/or robustness of the detection, identification, and/or predictive capacity of an iCAP system, composition, or method. In some cases, an indicator cell derived from an induced pluripotent stem cell (e.g., that has been derived from the subject or from a family member of the subject) may be advantageous to the accuracy or robustness of an iCAP system or method. In some cases, it may be advantageous to select or create a cell population or cell line that has been modified with an inducible expression system (e.g., a fluorescent protein-based reporter system, such as a doxycycline-inducible expression system, or a reporter system that is not fluorescence-based, such as a luciferase-based system) for use in an iCAP system or method, for example to more clearly measure a factor (e.g., a biomarker) in an assayed sample or expression of a gene of interest in an indicator cell population (e.g., a gene used as a response pattern feature or key response pattern feature in an iCAP system or method).

In some cases, a classifier, a computational model, or a method of training, validating or using a classifier of an iCAP system can be selected in order to optimize detection or evaluation of a specific physiological state or class of physiological states. For example, a physiological state wherein many samples are available for training of a classifier may include a neural networks, a decision tree (e.g., classification decision tree), and/or a k-nearest neighbor computational model so that large data sets may be handled efficiently and the classifier can be trained using a larger training and/or validation data set. If samples of a physiological state are in limited supply (e.g., because of a limited number of subjects exhibiting the physiological state, as may be the case for rare diseases, or because of technical difficulty involved in obtaining samples), it may be advantageous to include a support vector machine (e.g., Gaussian kernel or one-against-one), a naive Bayes, and/or a linear discriminant analysis module in a classifier of the iCAP system.

In some aspects, a lung cancer iCAP can be used as a blood-based test for patients with IPNs having a size (e.g., diameter) of 3 mm to 30 mm, 3 mm to 25 mm, 5 mm to 20 mm, 10 mm to 15 mm, no larger than 30 mm, no larger than 25 mm, no larger than 20 mm, no larger than 15 mm, no larger than 10 mm, no larger than 5 mm, no larger than 3 mm, less than 30 mm, less than 25 mm, less than 20 mm, less than 15 mm, less than 10 mm, less than 5 mm, or less than 3 mm (e.g., as identified by chest CT scan) to determine a physiological state in the one or more patients, e.g., to identify patients among the one or more patients with malignant nodules and/or benign nodules, potentially avoiding invasive biopsy in patients with benign nodules and/or identifying patients requiring such treatments. In some aspects, iCAP can be used as a test for patients with one or more nodules that have an intermediate risk of cancer (e.g., a 5% to 65% risk of cancer). In some cases, an iCAP system can be used as a test for patients with one or more nodules having a risk of cancer (e.g., malignancy) of from 5% to 70%, from 5% to 65%, from 10% to 60%, from 15% to 55%, from 20% to 50%, from 25% to 45%, or from 30% to 40%. In some embodiments, iCAP can be used in combination with a CT scan to improve early detection of lung cancer and/or to distinguish benign nodules from malignant nodules or nodules with high risk of developing lung cancer. Using iCAP to make such distinction can lower false positive rate and can avoid situations wherein patients with benign nodules are subjected to invasive and/or expensive follow-up tests.

In some aspects, compositions and methods disclosed herein are based on blood biomarkers (e.g., one or more factors present in a blood sample of a subject). The present disclosure also contemplates compositions and methods for diagnosing lung cancer using an indicator cell assay or a cellular response assay. In some cases, such cellular response assay can be used before or after a CT scan, or can be used in combination or in conjunction with a CT scan, e.g., to improve the accuracy of the diagnosis, to facilitate early diagnosis of lung cancer, and/or to reduce false positives of CT scans to prevent unnecessary follow-up procedures (e.g., biopsy).

In some embodiments, indicator cell assays can use standardized, cultured indicator cells, for example, which can interact differentially with a biological sample or fluid, such as serum, blood, or cell lysate, from normal tissue (e.g., tissue from a healthy subject) or immortalized cell source, which may be known not to have a negative or detrimental physiological state of interest (e.g., such as lung cancer), or which may be known not to have a high risk of having such a physiological state as compared to samples from diseased, abnormal, or unhealthy tissue (e.g., a tissue source known to have a negative or detrimental physiological state of interest, such as lung cancer, or which is known not to have a high risk of having such a physiological state). A cellular response of an indicator cell population (e.g., measured or detected values of parameters which comprise an iCAP response pattern feature set or iCAP key response pattern feature set) can capture or detect complex differences in samples. In some aspects, such cellular response assays provide greater sensitivity and specificity, especially when used in combination with existing diagnostic methods such as CT scans.

### Indicator Cells

An iCAP system or method can comprise one or more of a wide range of cell types that have known responsiveness to extrinsic signals of disease and disease-specific response signatures (which can comprise, for example, a set of key response pattern features, as described herein). Cells used in an iCAP system or method to generate a response pattern in response to a diseased or abnormal sample, such as serum from a subject, can be referred to as indicator cells.

As contemplated herein, indicator cells can be of one or more cell types that are capable of producing a response to a lung cancer cell or a lung cancer biomarker. A cell type or specific cell population can be selected for the reproducibility, robustness, and/or uniqueness of its response (e.g., a set of parameter values or changes in parameter values of the cell after contact with a sample) to one or more specific samples (e.g., samples derived from patients with a specific physiological condition, such as lung cancer).

iCAP systems, compositions, and methods for determining a physiological state of a subject (e.g., detecting lung cancer or determining a risk for lung cancer in a subject) disclosed herein can comprise one or more population of indicator cells. An indicator cell population can comprise one or more cells. An indicator cell population can comprise a plurality of cells. An indicator cell population can comprise one type of cell or two or more different cell types. In some cases, a first indicator cell population can comprise cells of the same source, type (e.g., phenotype or genotype), and/or disease state as one or more cells comprising a second, third, or additional indicator cell population. In some cases, a first indicator cell population can comprise cells of a different source, type (e.g., phenotype or genotype), and/or disease state as one or more cells comprising a second, third, or additional indicator cell population. In some embodiments, indicator cells (e.g., responder cells), can be used for a cellular response assay (e.g., an iCAP assay), comprising one or more cells (e.g., one or more indicator cells) capable of producing a change in 1 or more, 2 or more, 3 or more, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, or more than 10, more than 20, or more than 50, more than 100, more than 150, more than 200, more than 500, or more than 1000 cell response pattern features (e.g., cell parameters or biomarkers) when contacted with a sample. In some cases, a response pattern feature value (e.g., cell parameter value) of an indicator cell can comprise an expression level of a gene encoding a protein (or a concentration of the protein encoded by a corresponding gene) selected from epidermal growth factor receptor (EGFR), anaplastic lymphoma kinase (ALK), hepatocyte growth factor receptor (MET), ROS proto-oncogene 1 (ROS-1), Kirsten rat sarcoma viral oncogene homolog (KRAS), KIT proto-oncogene receptor tyrosine kinase (C-KIT), WASP family homolog 7 pseudogene (WASH7P), BRAF (V600E), HER2 (ERBB2), Janus kinase 2 (JAK2), programmed cell death protein 1 (PD-1), pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, C-reactive protein (CRP), hepatocyte growth factor (HGF), cancer/testis antigen 1B (NY-ESO-1), prolactin, ABL proto-oncogene 2, non-receptor tyrosine kinase (ABL2), adhesion G-protein coupled receptor G1 (ADGRG1), adrenoreceptor alpha 1B (ADRA1B), AKT serine/threonine kinase 3 (AKT3), alpha kinase 3 (ALPK3), ankyrin repeat domain 22 (ANKRD22), ankyrin repeat domain 37 (ANKRD37), armadillo repeat containing X-Linked 4 (ARMCX4), voltage-dependent calcium channel gamma-6 subunit (CACNG6), coiled-coil domain-containing protein 66 (CCDC66), cell migration-inducing and hyaluronan-binding protein (CEMIP), cardiotrophin 1 (CTF1), DEPP1, fatty acid hydroxylase domain containing 2 (FAXDC2), F-box and leucine rich repeat protein 5 (FBXL5), G protein-coupled receptor 17 (GPR17), hydroxyacylglutathione hydrolase-like protein (HAGHL), hypoxia inducible factor 1 (HIF1A or HIF1-alpha), interferon lambda 2 (IFNL2), interferon kappa (IFNK), interleukin 1 receptor 2 (IL1R2), kirre like nephrin family adhesion molecule 2 (KIRREL2), lysyl oxidase like 2 (LOXL2), mitochondrially encoded NADH:ubiquinone oxidoreductase core subunit 4 (MT-ND4), neural precursor cell expressed developmentally down-regulated protein 9 (NEDD9), PDZ domain containing protein 7 (PDZD7), protein kinase C alpha (PRKCA), proline-rich protein 22 (PRR22), periodic tryptophan protein 2 (PWP2), RAS protein activator like 1 (RASAL1), ring finger protein 223 (RNF223), receptor tyrosine kinase like orphan receptor 2 (ROR2), round spermatid basic protein 1 (RSBN1), solute carrier family 2 member 3 (SLC2A3), tripartite motif containing 2 (TRIM2), alanyl aminopeptidase (ANPEP), arylsulfatase A (ARSA), chromosome 2 open reading frame 69 (C2ORF69), caldesmon 1 (CALD1), chromobox 1 (CBX1), CAP-Gly domain containing linker protein family member 4 (CLIP4), collagen type VI alpha 1 chain (COL6A1), coenzyme Q4 (COQ4), dimethylarginine dimethylaminohydrolase 1 (DDAH1), disks large homolog 1 (DLG1), dual specificity phosphatase 6 (DUSP6), EPH Receptor B6 (EPHB6), family with sequence similarity member A (FAM72A), fibroblast growth factor 1 (FGF1), filamin A interacting protein 1 (FLIP1L), gap junction protein alpha 5 (GJA5), G-protein coupled receptor 143 (GPR143), interleukin 18 (IL18), laminin subunit alpha 1 (LAMA1), leptin receptor (LEPR), leucine rich repeat neuronal 4 (LRRN4), matrix metalloproteinase 9 (MMP9), myotubularin-related protein 10 (MTMR10), metallothionein 1F (MT1F), metallothionein 1M (MT1M), metallothionein 1X (MT1X), nuclear speckle splicing regulatory protein 1 (NSRP1), polo like kinase 2 (PLK2), pregnancy specific beta-1-glycoprotein 5 (PSG5), sphingosine-1-phosphate receptor 1 (S1PR1), surfactant associated 1 pseudogene (SFTA1P), solute carrier family 39 member 10 (SLC39A10), syntaxin 3 (STX3), sushi domain containing 2 (SUSD2), synaptopodin-2 (SYNPO2), transcription factor 25 (TCF25), transforming growth factor beta 2 (TGFB2), transmembrane 4 L6 family member 1 (TM4SF1), tripartite motif-containing protein 65 (TRIM65), tsukushin (TSKU), thioredoxin reductase 1 (TXNRD1), ubiquitin conjugating enzyme E2 J1 (UBE2J1), WW domain-containing adapter protein with coiled coil (WAC), WD repeat domain 13 (WDR13), metastasis-associated in colon cancer protein 1 (MACC1), chloride intracellular channel 4 (CLIC4), metallothionein 1E (MT1E), A-kinase anchor protein 12 (AKAP12), ephrin B2 (EFNB2), intersectin 2 (ITSN2), prolyl 4-hydroxylase subunit alpha 1 (P4HA1), pyruvate dehydrogenase kinase 1 (PDK1), stanniocalcin 1 (STC1), insulin growth factor-like family member 1 (IGFL1), serpin family B member 5 (SERPINB5), beta-1,4-galactosyltransferase 4 (B4GALT4), kruppel like factor 7 (KLF7), dysferlin (DYSF), interferon regulatory factor 6 (IRF6), tropomyosin 4 (TPM4), coagulation factor III (F3, tissue factor), SEC14 domain and spectrin repeat-containing 1 (SESTD1), bone morphogenetic protein 6 (BMP6), chromosome 1 open reading frame 74 (C1orf74), endoplasmic reticulum oxidoreductase 1 alpha (ERO1A), dihydrouridine synthase 1 like (DUS1L), ERBB receptor feedback inhibitor 1 (ERRFI1), procollagen-lysine, 2-oxogluarate 5-dioxygenase 2 (PLOD2), dickkopf related protein 1 (DKK1), nidogen-2 (NID2), lysine demethylase 6A (KDM6A), endothelin-1 (EDN1), TNF receptor superfamily member 10D (TNFRSF10D), oncostatin M receptor (OSMR), transferrin receptor (TFRC), Ras associated domain family member 3 (RASSF3), myristoylated alanine rich protein kinase C substrate (MARCKS), epithelial membrane protein 1 (EMP1), growth arrest specific 2 like 1 (GAS2L1), CUB domain containing protein 1 (CDCP1), Dnaj heat shock protein family Hsp40 member C3 (DNAJC3), SRY-Box transcription factor 4 (SOX4), golgi membrane protein 1 (GOLM1), serine incorporator 5 (SERINC5), lactate dehydrogenase A (LDHA), SPOC domain-containing protein 1 (SPOCD1), proline-serine-threonine phosphatase interacting protein 2 (PSTPIP2), Par-6 family cell polarity regulator beta (PARD6B, Partitioning defective 6 homolog beta), protein phosphatase 1 regulatory subunit 3B (PPP1R3B), hexokinase-2 (HK2), transmembrane protein 45A (TMEM45A), BTG anti-proliferation factor 1 (BTG1), pannexin 1 (PANX1), myosin VB (MYO5B), ankyrin repeat domain-containing protein 33B (ANKRD33B), sorting nexin 9 (SNX9), mortality factor 4-like protein 2 (MORF4L2), glial cell derived neurotrophic factor (GDNF), tripartite motif containing 58 (TRIM58), hematological and neurological expressed 1-like protein (HN1L, JPT2, Jupiter microtuble associated homolog 2), branched chain amino acid transaminase 1 (BCAT1), phosphodiesterase 8A (PDE8A), egl nine homolog 1 (EGLN1), keratin associated protein 2-3 (KRTAP2.3), solute carrier family 9 member a2 (SLC9A2), jun proto-oncogene, AP-1 transcription factor subunit (JUN), integrin subunit alpha 3 (ITGA3), Ras-related protein Rap-2b (RAP2B), SH3 domain containing kinase binding protein 1 (SH3KBP1), phosphoglycerate kinase 1 (PGK1), insulin induced gene 2 (INSIG2), cysteine rich C-terminal 1 (CRCT1), tumor associated calcium signal transducer 2 (TACSTD2), activated leukocyte cell adhesion molecule (ALCAM), torsin-1A-interacting protein 2 (TOR1AIP2), neuromedin B (NMB), trophoblast glycoprotein (TPBG), occludin (OCLN), threonyl-tRNA synthetase (TARSL2), sterile alpha motif domain containing 4A (SAMD4A), eukaryotic elongation factor selenocysteine-TRNA specific (EEFSEC), ATP binding cassette subfamily C member 4 (ABCC4), integrin subunit alpha V (ITGAV), aminopeptidase puromycin sensitive (NPEPPS), Ras-related protein Ral-A (RALA), AC006262.5, galectin-related protein (LGALSL), hydroxycarboxylic acid receptor 2 (HCAR2), solute carrier organic anion transporter family member 2a1 (SLCO2A1), formin homology 2 domain containing 1 (FHOD1), Rab GTPase-binding effector protein 2 (RABEP2), solute carrier family 25 member 37 (SLC25A37), vascular endothelia growth factor A (VEGFA), cadherin 1 (CDH1), insulin like growth factor binding protein 3 (IGFBP3), BRCA associated ATM activator 1 (BRAT1), family with sequence similarity 174 member B (FAM174B), PR/SET domain 1 (PRDM1), steroid sulfatase (STS), ubiquitin specific peptidase 53 (USP53), platelet endothelial aggregation receptor 1 (PEAR1), deleted in malignant brain tumors 1 (DMBT1), natriuretic peptide receptor 1 (NPR1), BCL2 interacting protein 3 like (BNIP3L), basic helix-loop-helix family member E40 (BHLHE40), midline 1 (MID1), cyclin G2 (CCNG2), lysine demethylase 3A (KDM3A), transmembrane protein 154 (TMEM154), noggin (NOG), kielin cysteine rich BMP regulator (KCP), KiSS-1 metastasis suppressor (KISS1), serine protease 22 (PRSS22), major histocompatibility complex class 1.V (HLA.V, HLA-V), ArfGAP with GTPase domain ankyrin repeat an PH domain 1 (AGAP1), apoptosis inhibitor 5 (API5), CCR4-NOT transcription complex subunit 11 (CNOT11), DnaJ homolog subfamily C member 5 (DNAJC5), exosome component 4 (EXOSC4), F-box protein 41 (FBXO41), integrin subunit beta 3 binding protein (ITGB3BP), Jrk helix-turn-helix protein (JRK), potassium calcium-activated channel subfamily M alpha 1 (KCNMA1), LETM1 domain containing 1 (LETMD1), long intergenic non-protein coding RNA 1588 (LINC01588), methyltransferase like 21A (METTL21A), mitochondrial ribosomal protein S15 (MRPS15), multivesicular body subunit 12A (MVB12A), Myb like SWIRM and MPN domains 1 (MYSM1), NAD kinase 2 mitochondrial (NADK2), NIPA magnesium transporter 1 (NIPA1), pirin (PIR), phospholipid transfer protein (PLTP), peptidylprolyl isomerase E (PPIE), protein phosphatase 1 regulatory subunit 12A (PPP1R12A), protein kinase C iota type (PRKCI), splicing factor 45 (RBM17), ring finger protein 24 (RNF24), sorting nexin 33 (SNX33), tubulin beta class I (TUBB), UL16 binding protein 2 (ULBP2), vestigial like family member 4 (VGLL4), tryptophanyl-TRNA synthetase 1 (WARS), WD repeat domain 45 (WDR45), zinc finger protein 318 (ZNF318) or any combination thereof. In some cases, a homolog of one or more of the genes listed herein is used, for example, if an indicator cell population (and, optionally, a sample) is from a non-human source (e.g., mouse, rat, cow, horse, cow, rabbit, bird, guinea pig, zebrafish, amphibian, cat, or dog). In some cases, the accuracy of a classifier used in the methods and systems disclosed herein can be improved when the classifier is used with a response pattern comprising one or more response pattern feature values selected from the genes encoding a protein selected from the group consisting of EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318.

A response pattern can comprise one or more response pattern features (e.g., cell parameters or biomarkers) that have been demonstrated to indicate the presence or absence of one or more physiological state or have been correlated with the presence or absence of one or more physiological state, such as a stage of lung cancer (e.g., pre-symptomatic, pre-clinical, stage I, stage II, stage III, or stage IV), or the absence of lung cancer (e.g., through statistical analysis of published or unpublished data).

In some cases, a response pattern or portion thereof can comprise one or more response pattern features (e.g., cell parameters or biomarkers) that have been identified as parameters indicative of a certain risk or range of risk for lung cancer. In some cases, a response pattern or portion thereof can comprise one or more response pattern features that, if present (e.g., if detected), if absent (e.g., if assayed but not detected), or if present at a level (e.g., detected or measured) above a threshold value, below a threshold value, or within a range of values, indicate a certain risk or range of risk for lung cancer. For example, a value of a response pattern feature (e.g., as measured from an indicator cell that has been contacted by a sample from a subject) can indicate a risk for lung cancer or another physiological state in a subject (e.g., the subject from whom the sample used to contact the indicator cell was derived) if the measured value of the response pattern feature is above a specific value, below a specific value, or within a range of values, e.g., that has been shown to indicate or has been correlated with the presence of lung cancer or another physiological state. In some cases, a response pattern or portion thereof can comprise one or more response pattern features that, if present (e.g., if detected) or if present above a value, below a value, or within a range of values, indicates that a subject does not have a physiological condition, such as lung cancer, or that the risk of lung cancer is no greater or no less than that of a larger population (e.g., a population that shares one or more demographic trait with the subject).

Although large quantities of time and resources have been expended in the search for biomarkers or cell parameters that can be reliably shown to indicate or have been reliably correlated with lung cancer (or another physiological state); very few such biomarkers or cell parameters have been identified. An advantage of many iCAP methods and systems described herein is that a set of response pattern features (e.g., the set of key response pattern features, which can be a subset of the features comprising the entire response pattern) that indicate or are correlated with the presence or absence of a physiological state, such as lung cancer, do not need to be known prior to the determination of the response pattern. In some cases, this means that identities of each response pattern feature (e.g., each cell parameter or biomarker) comprising the set of response pattern features sufficient to determine a subject's risk for a physiological state, such as lung cancer, (e.g., the set of key response pattern features, which can be a subset of the features comprising the entire response pattern) do not need to be known before the values for the features of the response pattern are determined. In some cases, iCAP systems and methods disclosed herein can be used as accurate and reproducible means of diagnosing and/or treating physiological states (e.g., diseases or risks of having a disease) that have recently been discovered and/or which may not be fully characterized (e.g., wherein a mechanism of action is not yet known to serve as the basis for a diagnosis or therapy, for example wherein subjects with and/or without the condition can be identified but symptoms or mechanistic pathways are not fully understood). In some cases, iCAP systems and methods can be used to determine that a subject has a novel physiological state or to determine a risk of the subject having a novel physiological state. In some cases, an iCAP system or method described herein can be used to determine if a patient has an infectious disease (e.g., a novel viral infection such as influenza, coronavirus, herpes, or a subtype or variant thereof or a novel bacterial infection or subtype or variant thereof) or to determine a risk of a patient having the infectious disease. In some cases, such systems and methods can provide a determination of whether a subject has the infectious disease (e.g., novel infectious disease) even when the infectious disease has not been identified mechanistically or fully characterized (e.g., through virus isolation). For example, an iCAP system or method can be used to determine the presence, absence, or risk of a novel physiological state (e.g., novel infectious disease or subtype or variant thereof) in a test subject, e.g., by using one or more samples from one or more positive control subjects known to have the novel physiological state (e.g., novel infectious disease or subtype or variant thereof) to contact a first indicator cell population, one or more samples from the test subject to contact a second indicator cell population, and, optionally, one or more samples from one or more negative control subjects known not to have the novel physiological state to contact a third indicator cell population (e.g., in the process of building and/or testing the iCAP system or method). The ability to use iCAP systems and methods in cases wherein one or more of (e.g., potentially all of) the parameters (e.g., features) sufficient and/or necessary to define a response pattern or key response pattern can be valuable when the response pattern is determined from one or more indicator cell population (e.g., by measuring values for the response pattern features of the response pattern), as interactions between the cells of an indicator cell population and the sample(s) with which the cells are contacted can be very unpredictable. For example, if the type(s) of cells comprising an indicator cell population contacted with a sample to produce a response pattern do not normally come into contact with the type of sample (e.g., directly from the subject or after processing performed on the sample after extraction from the subject) with which they are contacted (e.g., under physiological or laboratory settings), the response of the cells to the sample will likely be difficult to predict beforehand. For example, many embodiments of iCAP systems and methods comprise contacting an indicator cell population with a sample that they would never contact in vivo. In many embodiments, it is likely that a set of key response pattern features that are sufficient to indicate a risk for a physiological state (e.g., the presence, absence, or stage of lung cancer) has not been previously determined for the indicator cells and sample used (e.g., for reasons including those described above).

A response pattern or portion thereof (e.g., a set of response pattern features of the response pattern) can comprise one or more response pattern features (e.g., cell parameters or biomarkers). A response pattern or portion thereof (e.g., a set of response pattern features of the response pattern) can comprise one or more features (e.g., parameters or biomarkers) that have not been specifically identified as parameters indicative of a risk for lung cancer (or another physiological state). In some cases, methods and systems disclosed herein comprise determining a set of response pattern features (e.g., a set of key response pattern features) that indicate the presence, absence, and/or risk for a physiological state, such as one or more stages of lung cancer (e.g., pre-symptomatic lung cancer, pre-clinical lung cancer, stage I lung cancer, stage II lung cancer, stage III, lung cancer, or stage IV lung cancer). For example, methods and systems disclosed herein can comprise determining a set of key response pattern features based on one or more response pattern feature values of one or more response patterns. As described herein, a set of key response pattern features (which can be a subset of the features comprising a response pattern) can be determined from one or more response patterns (e.g., using one or more classifier, e.g., as described herein). In some cases, a subject's risk for lung cancer can be determined based on a set of key response pattern features. In some cases, a subject's risk for lung cancer can be determined based on a set of key response pattern features and a response pattern or portion thereof (e.g., comprising one or more response pattern feature values for one or more features of the set of key response pattern features) that has been determined, at least in part, by measuring one or more features (e.g., cell parameters or biomarkers) of an indicator cell population contacted by a sample from the subject.

One or more cell parameter (e.g., biomarker) can comprise a feature of a response pattern. In some cases, a response pattern feature value can be a cell parameter value (e.g., a biomarker value). In some cases, a response pattern feature value comprises one or more of: an epigenetic pattern, a gene expression level, an RNA abundance level (which can, for example, result from an RNA transcription level and RNA splicing levels), an intracellular protein concentration, a concentration of a low molecular weight metabolite, or a concentration of a secreted protein or a cell surface protein. A response pattern feature value (e.g., a response pattern feature value of a first, second, third, or one or more additional subject, a differential response pattern feature value, and/or a key response pattern feature value) can be measured or determined using one or more of the experimental techniques or assays disclosed herein. In many cases, a response pattern feature value can be measured or otherwise determined (e.g., from an assay or technique disclosed herein) after an indicator cell has been contacted with a sample from a subject.

In some embodiments, an indicator cell population comprises a clonal cell or a plurality of clonal cells derived from stem cells. In some embodiments, a population of indicator cells is a mixture of a plurality of different clonal cell populations. In some cases, an indicator cell population (e.g., comprising a plurality of indicator cells) of an iCAP system, kit, or method may be useful in responding to one or more different factors in a sample. For example, each indicator cell type of an indicator cell population comprising a plurality of indicator cell types may be responsive to one or more substances indicative of the presence (or absence) of lung cancer. In some embodiments, indicator cells produce a change in one or more value of a cell parameter (e.g., which may comprise a portion of the feature(s) of a response pattern) when contacted by a sample. In some cases, a set of one or more cell parameters can be used to detect, distinguish, classify, or diagnose the presence of lung cancer or a risk of lung cancer in a biological sample from a subject.

Examples of indicator cells can include, but may not be limited to, primary cells; immortalized cells; or cultured or engineered cells derived from stem cells, progenitor cells, or induced pluripotent stem cells; partially differentiated cells; or terminally differentiated cells. In some embodiments, indicator cells can be physically incorporated into a system or kit, or a method (e.g., cells can be cultured in a vessel of the system or method). In some embodiments, indicator cells used in iCAP lung cancer include, but are not limited to, lung epithelial cells, epithelial cell line cells, and endothelial or epithelial cells derived from induced pluripotent stem cells.

Immune cells can be used as indicator cells in the systems, compositions, and methods described herein for determining a physiological state like lung cancer. In some embodiments, indicator cells can include immune cells, such as lymphocytes, B cells, and/or T cells. In some embodiments, immune cells, e.g., lymphocytes, T cells, B cells, CAR-T cells, can be engineered to be responsive to one or more substance indicative of lung cancer.

In some embodiments, an indicator cell can be a fibroblast. In some cases, an indicator cell can be an endothelial cell. In some cases, an indicator cell can be a lung cell (such as an alveolar cell or a lung epithelial cell), an immune cell, or a combination thereof. In some embodiments, lung cancer indicator cells can be a clonal cell population that is responsive to lung cancer or a substance indicative of lung cancer. In some embodiments, an indicator cell can be an engineered cell, a cultured cell, a cell of a cell line (e.g., an immortalized cell), a cell derived from an animal model, or a cell derived from a human cell.

In some embodiments, indicator cells can be of a cell type that is known to be relevant or affected by lung cancer, such as tracheal cells, epithelial cells (e.g., bronchial epithelial cells), smooth muscle cells, alveolar cells, and pneumocytes.

Surprisingly, non-immune cells and/or cells not known to be directly affected by a physiological state (e.g., lung cancer) can be excellent indicator cells in systems and methods disclosed herein even though they may not be understood to respond to a physiological state of interest by producing a representative or reproducible response pattern. For example, cells used in systems, compositions, kits, or methods of use in determining a physiological state of a sample or subject as disclosed herein can be a general stromal cell (e.g., a fibroblast) or a specialized cell (e.g., an epithelial cell, or an endothelial cell). In some cases, indicator cells, or any derivative or cell line derived therefrom, can respond to factors in a sample (e.g., substances indicative of the presence of lung cancer or substances indicative of the absence of lung cancer, such as proteins and/or nucleic acids) to yield differential response patterns that can be measured directly or indirectly to determine patterns related to or indicative of a disease (e.g., lung cancer) or a disease stage (e.g., the extent to which a disease has progressed, which can be represented by a defined stage of cancer). In some cases, the use of non-immune cells as indicator cells can be advantageous in that they may be less expensive or technically difficult to procure, maintain, or use in systems, compositions, or methods disclosed herein.

In some embodiments, indicator cells can be cultured, engineered, cloned, or immortalized. In some embodiments, indicator cells can be clonal cell population derived from stem cells, such as endothelial cells derived from induced pluripotent stem cells (iPSCs) or lung epithelial progenitor cells derived from embryonic and induced pluripotent stem cells. In some embodiments, indicator cells can be derived from animal models or from human cells. In some embodiments, indicator cells can be alveolar cells, lung epithelial cells, endothelial cells, immune cells, or a combination thereof. In some embodiments, indicator cells can be capable of multicomponent gene expression readout.

A cell parameter of indicator cells that can be measured, detected, or analyzed in a system, kit, or method described herein can be an identity, quantity, or change in quantity of a fluid, peptide, polypeptide, nucleic acid, oligonucleotide, ion, enzyme, or other cellular product produced by the indicator cell. In some cases, a cell parameter is measured, detected, or analyzed while the cell is intact. In some cases, a cell parameter can be measured, detected, or analyzed after the cell is no longer intact (e.g., as an extract of a cell). As described herein, a cell parameter can be a feature of response pattern or key response pattern of an iCAP system or method.

In some instances, cells can be used to produce fluids, peptides, polypeptides, nucleic acids, oligonucleotides, ions, enzymes, or other cellular products. Cells (or cell lines or derivative products thereof) can be modified, differentiated, genetically manipulated or engineered, stimulated, inhibited, or fragmented, and/or isolated prior to or during incorporation into the iCAP system or methods of use thereof.

In some embodiments, indicator cells can be selected from cells that are responsive to changes in compositions associated with an abnormal or diseased condition. For example, conditions that are associated with abnormalities in the lung, lung epithelial cells and/or alveolar cells may be used as indicator cells or indicator cell lines or cultures in some embodiments.

In some embodiments, identification of optimal indicator cells can be accomplished by running the iCAP system or method of use thereof with standard conditions using three indicator cell types including: 1) two types of normal large-airway epithelial cells, and 2) endothelial cells differentiated from iPSCs (e.g., which may be especially responsive to tumors during malignant transformation). For each cell type, iCAP analysis can be performed using aliquots of serum from the same subject or group of subjects, RNA-seq, genome alignment and analysis of differential expression between test samples (e.g., case or patient samples, for example, wherein subject from which the test sample is obtained has an unknown physiological state at the time of sample collection) and control samples.

To identify the optimal indictor cells, iCAP expression profiles of candidate cell types can be compared to identify cells that show characteristics for: 1) maximizing the number of significantly differentially expressed genes and magnitude of differential expression, 2) minimizing median intra-class coefficient of variation (CV) to reduce noise in the assay, and/or 3) maximizing significant enrichment of lung cancer-related gene sets amongst the differentially expressed genes.

In some embodiments, identification of optimal parameters of the assay (e.g., optimal indicator cells or optimal parameters for incubation of a patient sample with indicator cells) can include performing an iCAP assay under various conditions with pooled samples from case subjects and pooled samples from control subjects. In some cases, identification of optimal parameters (e.g., optimal conditions) of the assay can include measuring the levels of response pattern features (e.g., response pattern features that have been shown previously to indicate the presence or absence of the physiological state in the iCAP). In some cases, optimal parameters can be identified as the conditions resulting in maximal magnitude and/or maximal statistical significance of differential abundance of the response pattern features.

For assessment of abnormal conditions (e.g., diseases such as lung cancers like small cell lung cancer, non-small cell lung cancer, mesothelioma, or carcinoid tumors) that exhibit their biological effects on the pulmonary system, lung cells may be used. In some embodiments, a cell type different from the diseased cell type that shows a sufficiently nuanced pattern in response to an abnormal condition can be used as indicator cells.

In some embodiments, iCAP systems or methods can comprise the production of a differential response pattern from lung epithelial cells contacted with serum from different subjects (e.g., patients) with IPNs. In some cases, a differential response pattern can be used to diagnose a subject as having lung cancer or benign nodules.

In some embodiments (e.g., where conditions are not directly specific to a particular type of cell or where malignancy affects multiple cell types), indicator cells can be selected from cell types known to be associated with the disease or cancer. In some cases, such cells can be selected from normal tissue affected by the disease or cancer.

In some embodiments, indicator cells can be selected from primary target cell types of a disease or cancer, such as lung epithelial cells for iCAP directed to lung cancer. In other embodiments, indicator cells may not be primary target cell types of a disease or cancer, but may be capable of responding to changes in the target cell types of a disease or cancer.

In some embodiments, iCAP system or method of use thereof for diagnosing lung cancer, as described herein, can comprise indicator cells, which can be cultured from or derived from stem cells or progenitor cells. A stem cell can be a cell with the capacity to differentiate into more than one cell type (e.g., produce daughter cells of a different phenotype or epigenetic state). A stem cell can be a renewable cell with the capacity to produce daughter cells indefinitely. A progenitor cell can be a cell with the capacity to differentiate into more than one cell type. Stem cells and progenitor cells can be identified by functional or structural characteristics, such as epigenetic marking, genetic activity, or nucleic acid conformation (e.g., histone modifications, chromatin conformation, gene expression, protein expression, transcription factor expression, etc.).

Angiogenesis or blood vessel formation can be a fundamental step in malignant tumor formation and is also associated with other health concerns including proliferative retinopathy associated with diabetes, and ischemia associated with stroke and heart disease. It can be mediated by mobilization and recruitment of bone marrow-derived endothelial precursor cells (including endothelial progenitor cells (EPCs), and hematopoietic stem and progenitor cells. Recruitment of these cells to the target location can be mediated by signaling molecules in the serum (including cytokines, angiogenic factors, platelet-derived growth factors, and as of yet uncharacterized factors), which comprise organ-specific signatures. Upon reaching the target tissue, differentiation of the progenitor cells can be influenced by specific signaling molecules including local extracellular matrix components. Therefore, a potential biosensor assay for tumor development and other cell proliferative conditions can include the use of detector cells that are EPCs and other vascular progenitor cells (which can be isolated from bone marrow, or derived from embryonic stem cells), and the use of blood serum as the biofluid.

In some embodiments, iCAP assay can be used to detect and characterize tumors by detecting or responding to secretion of proteases (both matrix metalloproteases and serine and threonine proteases) or molecules from tumor cells. For example, certain proteases that break down extracellular matrix components and release locally confined growth factors and polysaccharides that regulate cell behavior into the blood stream can be detected by indicator cells or indicator cells that come in contact with a sample of the blood.

A response pattern feature value (e.g., biomarker value) can include any measurement indicative of an interaction between a biological system and a risk for lung cancer, which may be chemical, physical, or biological. The measured response (e.g., value) for a response pattern feature (e.g., biomarker) can be functional, physiological, biochemical at the cellular level, or a molecular interaction. Examples of response pattern features (e.g., biomarkers) can include, but may not be limited to, blood pressure, medical history, smoking status, age, serological marker, a gene, a protein, a metabolite, a cell, a receptor, cell-surface marker, oncogene, antibodies, immunoglobulin, etc. A response pattern feature (e.g., biomarker) can be measurable and/or detectable and contributes to one's assessment of a lung cancer risk. In some cases, one or more value for a response pattern feature or a plurality of response pattern features can be a portion of a response pattern or differential response pattern of the indicator cells (e.g., in response to being contacted with a sample of a subject, such as a biological fluid). In some cases, one or more value for a response pattern feature or a plurality of response pattern feature can be the entirety of a response pattern or differential response pattern of the indicator cells in response to biological fluid(s) of subject(s).

In some cases, it may be advantageous to inhibit differentiation of a cell. For example, an iCAP system can be used to study response patterns using cancer stem cells as an indicator cell. In some cases, stem cell differentiation can be arrested or inhibited. Inhibition or arrest of cell differentiation can be accomplished in various ways, including the application or the deprivation of chemical, mechanical, electrical, or magnetic stimuli. In some cases, inhibition or arrest of differentiation can be accomplished by genetic engineering of a cell.

In some embodiments, iCAP system or method of use thereof for diagnosing lung cancer, as described herein, uses immortalized cells, cell line, or culture. An immortalized cell can result from natural mutagenesis or induced mutagenesis (e.g., through the use of chemical reagents or mutagens or through genetic engineering strategies, which can include delivery of peptides, proteins, or nucleic acids through viral vectors or plasmids). An immortalized cell can produce identical cells (e.g., through mitosis) indefinitely.

In some cases, immortalized cells or cell lines can be derived from naturally occurring cancer cells. In other embodiments, methods for generating immortalized cells can include introducing a viral gene that deregulates cell cycle, introducing an expression construct that expresses proteins that induce immortality, or hybridoma technology for generating immortalized antibody-producing B cells. In some cases, established immortalized cell lines can be used for iCAP assays, including, but not limited to, HBEC3-KT (which are derived from normal lung tissue), NuLi-1 cells (which are derived from normal lung tissue), 16HBE, MRC5, 3T3 cells, A549 cells (which are derived from lung tumor of a cancer patient), HeLa cells, HEK 293 cells, and Jurkat cells.

Indicator cells used in the systems and methods described herein can be modified in various ways to enhance their function as indicator cells. In some cases, cells (e.g., indicator cells) can be genetically engineered, chemically stimulated, mechanically stimulated, electrically or magnetically stimulated, fragmented, or differentiated.

In some cases, cells can be genetically engineered to comprise a certain genotype or phenotype. For instance, a cell can be engineered through viral, non-viral, chemical transfection, transformation, or transduction methods. In some cases, transfection reagents, such as FuGene®, HeLaMONSTER®, or Lipofectamine®, or chemicals such as calcium phosphate, can be used to alter cells.

In some embodiments, a cell can be modified to express, to contain, or to be associated with a detectable marker. Engineering of a cell can involve genome editing, which can involve homologous recombination, CRISPR/Cas-based systems, zinc-finger nucleases, or TALENs. Delivery of cellular or genetic engineering reagents can involve viral vectors, plasmids, transfection reagents, or electroporation.

In some embodiments, the transcription and/or translation products of the cell can be a cell parameter (e.g., biomarker or detectable marker) detected, measured, identified, or analyzed (e.g., in the methods and use of systems disclosed herein). In some embodiments, the transcription and/or translation products can be produced from an exogenously introduced nucleic acid cassette (such as a plasmid, a single-stranded RNA oligonucleotide, a non-coding RNA, a single-stranded DNA oligonucleotide, an RNA/DNA hybrid, or a double-stranded DNA oligonucleotide). In some embodiments, transcription and/or translation products (e.g., elements or signals that generate a response pattern) can be produced from a endogenous nucleic acid (e.g., a nucleic acid present in the original cell, stem cell, progenitor cell, or immortalized cell or a nucleic acid produced or replicated from the nucleic acid present in the original cell, stem cell, progenitor cell, or immortalized cell).

A parameter of an indicator cell (e.g., a biomarker or feature of an indicator cell) can be detected, measured, identified, or analyzed using one or more analytical method or technique. Metrics used to measure parameters of the systems and methods disclosed herein can include one or more of: radiation intensity (e.g., light intensity), radiation frequency (e.g., frequency or wavelength of light), mass (e.g., mass of a protein or nucleic acid), concentration, activity (e.g., enzymatic activity, binding efficiency, inhibition activity), size (e.g., height, width, length, depth, thickness, radius of curvature, diameter, perimeter, radius, surface area, cross-sectional area, volume), location (e.g., spatial proximity, spatial distribution), density, viscosity, refraction index, shape, and quantity. In some cases, a parameter of an indicator cell can be quantified (e.g., for use as a response pattern feature value in an iCAP system or method). Quantification of a parameter of an indicator cell (e.g., a biomarker) can be binary (e.g., present or absent), discrete (e.g., as represented in data with discrete increments or quantities), or continuous (e.g., as represented in data that can be represented with precision at least equal to that of the measurement method).

Analytical methods may be used to detect, measure, identify, or analyze a parameter of the systems and methods disclosed herein. Representative examples of methods and techniques used to detect, measure, identify, or analyze parameters or to obtain substances (e.g., isolated or purified proteins or nucleic acids), for example from indicator cells or an extract thereof, can include: microscopy (including fluorescence microscopy, confocal microscopy, electron microscopy, light microscopy), mass spectrometry, electrophoresis (e.g., capillary electrophoresis, gel electrophoresis), chromatography (e.g., gas chromatography), colorimetry, polymerase chain reaction (e.g., qPCR, RT-PCR, rolling circle PCR, isothermal PCR), migration assays, colony formation assays, enzymatic assays, ELISA, flow cytometry, cytotoxicity assays, proliferation assays, phagocytosis assays, immunoprecipitation, Western blot assays, Northern blot assays, Southern blot assays, and a combination of one or more thereof.

### Classifiers

A classifier disclosed herein can be a tool used (e.g., in an iCAP system or method) to determine or clarify the class or category of a subject, the class or category of a subject's condition (e.g., risk for lung cancer), and or the class or category of one or more assessed aspect of a subject's condition (e.g., risk of one or more nodules for malignancy). In some cases, a determination produced by or informed by a classifier can be based at least in part on values of data points (e.g., comprising all or a portion of clinically-assessed and/or non-clinically assessed data obtained via one or more evaluations or tests) and/or data about the subject (e.g., background and/or biographical data), which can be included in the classifier as classifier features. The features used by the classifier can be a specific set of features (e.g., key features) that are inferential about the class of a subject. For example, a classifier to predict a presence of cancer in a subject may include tumor size (e.g., diameter) as a classifier feature. In some cases, a classifier disclosed herein may predict the class (e.g., physiological state) of a subject (e.g., risk for the subject having a cancer, such as lung cancer) by comparing the values of features of that subject with the values of the same features from one or more other subjects (e.g., wherein the classes of the one or more other subjects are known). For example, a system or method comprising a classifier disclosed herein may predict the risk of a subject having a condition (e.g., lung cancer) at least in part by comparing the values of features determined from the use of a sample from that subject with the values of the same features determined from the use of a sample (or plurality of samples) from one or more other subjects (e.g., wherein the physiological state(s) of the one or more other subjects is known).

In some cases, a classifier can comprise a computational model and/or a means of creating a computational model. Disease classifiers disclosed herein can be developed using a method comprising, in part, one or more machine learning approaches. In some aspects, machine learning can be a computer-based process, which can comprise generating and, optionally, testing various computational models (e.g., for use in a classifier system), whereby the performance of the preceding tests are used to modify the parameters of the next test. In some aspects, developing a classifier using machine learning can involve the use of a training set of data pertaining to one or more subjects or subjects' conditions (for example, wherein the class is known) and tested using a held-out validation or test set of samples (e.g., wherein the held-out validation or test set data comprises data where the class is known but blinded). In some cases, a classifier's performance can be evaluated by how frequently it predicts the correct classes of the blinded samples. For example, in some cases, a first classifier (e.g., a classifier disclosed herein or system comprising a classifier disclosed herein) for determining or predicting a class (e.g., a state, risk, or condition of a patient or nodule) may be considered to perform better than a second classifier or second system if the first classifier correctly identifies positive results (e.g., high risk, affected, or diseased subjects) more frequently than a second classifier. In some cases, a first classifier (e.g., a classifier disclosed herein or system comprising a classifier disclosed herein) for determining or predicting a class (e.g., a state, risk, or condition of a patient or nodule) may be considered to perform better than a second classifier or second system if the first classifier correctly identifies negative results (e.g., low risk, unaffected, or healthy subjects) more frequently than a second classifier. In some cases, a first classifier (e.g., a classifier disclosed herein or system comprising a classifier disclosed herein) for determining or predicting a class (e.g., a state, risk, or condition of a patient or nodule) may be considered to perform better than a second classifier or second system if the first classifier correctly identifies positive results (e.g., high risk, affected, or diseased subjects) and negative results (e.g., low risk, unaffected, or healthy subjects) more frequently, in sum, than a second classifier. In some cases, a first classifier (e.g., a classifier disclosed herein or system comprising a classifier disclosed herein) for determining or predicting a class (e.g., a state, risk, or condition of a patient or nodule) may be considered to perform better than a second classifier or second system if the first classifier correctly identifies both positive results (e.g., high risk, affected, or diseased subjects) more frequently than a second classifier and negative results (e.g., low risk, unaffected, or healthy subjects) more frequently than a second classifier.

Machine learning approaches for classifier development can use multiple features from each subject and the relationship or set of relationships between features to predict the class of a sample. In some cases, a machine learning approach can utilize ensemble methods (e.g., wherein classification is based on the results of several different tests). Models for classification generated by machine learning approaches can be complex and non-intuitive, and methods and systems utilizing or generated in part by using one or more machine learning approaches often achieve performance not otherwise attainable by other means. For example, a system or method comprising or generated using ensemble methods can achieve performance not otherwise attainable by other means, including some systems or methods comprising or generated using a single machine learning method or test.

Systems, compositions, and methods for determining a physiological state of a subject (e.g., determining or detecting the presence of, absence of, or risk for a lung cancer) can comprise one or more classifiers. A classifier can be used to analyze, parse, integrate, or classify data from one or more experiment in which an indicator cell is contacted with a sample. In some cases, data used by a classifier (e.g., to generate a response pattern or in the determination of the presence or absence of lung cancer, in accordance with systems, compositions, and methods described herein) can comprise one or more response pattern feature values (e.g., indicator cell parameters or biomarkers). In some cases, data used in a classifier comprises one or more response pattern feature values (e.g., indicator cell parameters or biomarkers) differentially expressed when contacted with a first sample vs. indicator biomarkers expressed when contacted with a second sample. In some embodiments, indicator cell features (e.g., response pattern features determined from an indicator cell population) used in a classifier can comprise one or more gene expression levels, one or more methylation states, one or more protein production levels, one or more protein activity levels, one or more nucleic acid transcription or degradation rates, one or more quantities or relative abundances of a nucleic acid, or data indicating spatial localization of one or more protein and/or one or more nucleic acid (e.g., spatial position inside of or on the outer membrane of a cell).

Data analyzed by a classifier can comprise metadata. In some cases, a classifier of the systems, compositions, and methods disclosed herein can be used to analyze, parse, integrate, or classify all or a portion of the data comprising a response pattern (e.g., one or more features of a response pattern). In some cases, a classifier can be used to produce a differential response pattern (e.g., by analyzing, parsing, integrating, or classifying all or a portion of the data comprising a response pattern). In some cases, the data of a response pattern does not have similar statistical characteristics (e.g., similar variances, averages, sizes, etc.) as data used to produce the response pattern (e.g., a set of indicator cell biomarkers). It can be advantageous (e.g., in terms of processing speed, required processing power, or accuracy of classifier results) to use a different type of classifier to analyze data sets having different statistical characteristics.

Data used by a classifier can comprise medical history data, data indicating gender, data indicating age, smoking history or status data, co-morbidity data, diagnostic imaging data (such as CT scan data, Mill data, ultrasound data, X-ray data), or data indicating a size, shape, texture, spatial position, density of a lesion or nodule, or number of nodules present. In some cases, a size of a lesion or nodule can be a diameter, a length, a width, a depth, a perimeter length, a circumference, a surface area, a cross-sectional area, a volume of the lesion or nodule. In some cases, a shape of a lesion or nodule can comprise a surface feature or texture.

Data used by a classifier can also comprise data obtained from cancer cells themselves (e.g., cancer cell lysate, biomarkers expressed by cancer cells, or substances secreted by a cancer cell, including but not limited to expression levels, quantities, or activity levels of nucleic acids or proteins).

Systems, compositions, and methods disclosed herein can comprise a plurality of classifiers. In some cases, a plurality of separate classifiers can be used to analyze separate sets of data. For example, separate classifiers of the same type can be used to analyze data from separate indicator cell experiments (e.g., separate indicator cell experiments involving different subject samples). In some cases, separate classifiers of different types can be used to analyze data from separate indicator cell experiments. In some embodiments, at least one, two, three, four, five, six, seven, eight, nine, or ten classifiers are used to evaluate a test sample. In some embodiments, two or more, three or more, four or more, five or more, six or more, seven or more, eight or more, nine or more, ten or more, or more than ten, more than fifteen, or more than twenty classifiers are used to evaluate a response pattern, make a diagnosis, or to treat a subject based on the response pattern.

In some cases, an ensemble classifier (e.g., a classifier comprising two or more classifier modules) can be used to analyze, parse, integrate, or classify data produced or received in the systems, compositions, or methods disclosed herein. In some cases, an ensemble classifier (e.g., a sequential ensemble classifier) can pass analyzed data from a first classifier module of the ensemble classifier to a second classifier module of the ensemble classifier for subsequent analysis. In some cases, an ensemble classifier (e.g., a parallel ensemble classifier) passes portions of a data set to separate classifier modules of the ensemble classifier and analyzed individually. An ensemble classifier can be a homogenous ensemble classifier (e.g., a classifier having a plurality classifier modules of the same type) or a heterogeneous ensemble classifier (e.g., a classifier comprising a plurality of classifier modules of different types).

An ensemble classifier can provide improved predictive power in the systems, compositions, and methods for determining a physiological state disclosed herein. For example, a heterogeneous ensemble classifier comprising a first classifier module having low variance (e.g., linear regression models, linear discriminant analysis models, or logistic regression models) and a second classifier module having low bias (e.g., decision tree classifiers, k-nearest neighbor classifiers, and support vector machines (SVM)) can provide improved predictive power. A representative example of an ensemble classifier useful in the systems and methods disclosed herein is the random forest classifier.

In some cases, an ensemble classifier for use in the systems and methods disclosed herein can comprise a meta-model (e.g., through classifier stacking). Training a meta-model of a classifier for use in the systems and methods disclosed herein can comprise training a first classifier on a first dataset, training a second classifier on a second dataset, and training the meta-model on the output of the first and second classifiers (e.g., after the first and second classifiers have been trained). A meta-model can be trained on a plurality of classifiers. A first and second classifier of a meta-model can be different classifiers.

The selection of which type or types of classifier(s) used to analyze a given data set can be informed by the characteristics of data to be analyzed. Data sets comprising a plurality of samples derived from one or more subjects often reflect high variability both with respect the factors present in the sample (e.g., biomarkers present in a blood sample) and with respect to the relative abundance of each factor in each sample. A notable advantage of the systems, compositions, and methods described herein is that data sets having high variance can be efficiently and accurately evaluated in the determination of a physiological state, e.g., in the detection of a lung cancer.

Various embodiments of the systems, compositions, and methods described herein can be used to determine a physiological state (e.g., detect a lung cancer) from small or large data sets efficiently and with reliable accuracy. A large dataset can be a dataset comprising data obtained across multiple systems, which may comprise individual datasets that do not include values for all features being analyzed by the classifier (e.g., all features of a response pattern) or which comprise multiple datasets that have substantially different variances. In some cases, the size of a dataset can depend on the type(s) and/or complexity of classifier being used. For example, a dataset may be considered large for a classifier that requires large amounts of processing power to execute or train and considered small for a classifier that does not require large amounts of processing power to execute or train. In some cases, the size of a dataset depends on the processing power of the computer system on which the classifier is trained or used. For example, a dataset may be considered large when a classifier is trained or used on a standard desktop computer but may be considered not to be large if the classifier is trained or used on a supercomputer. In some cases, the size of a dataset depends on the number of categories or features of the dataset. In some cases, a large dataset comprises at least 100, at least 1,000, at least 10,000, at least 20,000, at least 30,000, at least 40,000, at least 50,000, at least 100,000, at least 1,000,000, or at least 1,000,000,000 categories or features. In some cases, a small dataset comprise at most 1, at most 2, at most 3, at most 4, at most 5, at most 6, at most 7, at most 8, at most 9, at most 10, at most 15, at most 20, at most 25, at most 30, at most 35, at most 40, at most 50, at most 60, at most 70, at most 80, at most 90, at most 100, at most 1,000, at most 10,000, from 25 to 35, from 20 to 30, from 30 to 40, from 20 to 40, from 10 to 50, or from 1 to 100 categories or features.

A classifier used herein can be supervised, semi-supervised, or unsupervised. A supervised classifier may be trained (e.g., built or developed) by providing the classifier with known inputs (e.g., one or more response patterns from positive and/or negative control samples) and labels for the known inputs (e.g., providing information to the classifier with respect to the identity of the variables, metrics, or features of the input). In some cases, supervised classifiers are trained with one or more training datasets of known inputs with labels identifying the category (e.g., positive control or negative control) in which the training dataset (e.g., the set of features, which can comprise biomarker data) belongs. Training of the classifier can also comprise providing the classifier with one or more validation datasets, which may be provided to the classifier to determine the accuracy and robustness of the classifier's prediction ability. In some cases, the use of validation datasets can be useful in signaling when a supervised classifier is overtrained (e.g., when prediction error rate begins to increase). Training of a classifier can also comprise providing the classifier with a holdout dataset (e.g., a dataset that has not been provided to the classifier as either a training dataset or validation training set). In some cases, training of a classifier comprises using a holdout dataset as a final validation dataset. A supervised classifier can offer advantages to defining the how many and which types of features are to be included in a response pattern or differential response pattern (e.g., which may later be used to classify a response pattern from a test sample) in when incorporated into systems and/or methods for determining a physiological state of a subject or sample. Advantageously, supervised classification approaches can allow the user to define case (e.g., “unknown” or “experimental”) and control (e.g., positive control and/or negative control) classes and direct the analysis to identify differences between the defined classes. In contrast, unsupervised approaches can categorize or group samples based on the strongest differential patterns across all the samples without regard to the subject classes. Therefore, whereas unsupervised approaches can be useful in exploring data space and identifying potential features useful for disease classification, supervised approaches can be used for training a model or classifier with features (e.g., such as those features identified using an unsupervised approach).

The predictive power of methods and systems described herein can be compared to the predictive power of other techniques. For example, a holdout dataset and/or a dataset from one or more test subjects can be independently scored by using manual or known methods or systems and by using a method or system described herein and then comparing the accuracy of the predicted results using each method versus the true result, which may be known beforehand (e.g., as with a holdout dataset) or which may be determined subsequently (e.g., as can be the case for subject-derived data). In many cases, the predictive power of various methods and systems described herein is statistically superior to manual or known techniques. In some cases, the statistical significance of the improvement in predictive power of various methods and systems described herein over existing methods and systems is reflected by a p-value of less than 0.1, less than 0.05, less than 0.01, less than 0.005, less than 0.001, less than 0.0005, or less than 0.0001.

The number of rounds of training and/or validation used to train a classifier (e.g., a supervised classifier) can be influenced by the availability of response patterns to be used as training or validation datasets. In some cases, a greater number of rounds of classifier training may be preferable to a fewer number of rounds of training. In some cases, a classifier can be overtrained by too many rounds of training (e.g., as can be the case with decision tree classifiers). In some cases, classifier training can be ended after additional rounds of training result in increased error in the classifier's accuracy (e.g., as determined through validation). In some cases, classifier training can be ended when the increase in accuracy error between training rounds is at least 0.01%, at least 0.1%, at least 0.5%, at least 1.0%, at least 1.5%, at least 2.0%, at least 3%, at least 4%, at least 5%, at least 10%, at least 15%, from 0.01% to 1%, from 1% to 5%, from 5% to 15%, or greater than 15%. In some cases, prevention of overtraining of a classifier or overfitting of data can be accomplished with top-down or bottom-up decision tree pruning (e.g., through reduced error pruning or cost complexity pruning, which can remove portions of a decision tree constructed by a classifier that contribute relatively little additional classification power to the decision tree) or by defining a specific number of rounds of training for the classifier (e.g., after which point the classifier is no longer subjected to training). Using a random forest ensemble classifier in place of a decision tree classifier can also help to prevent overfitting of data. In some cases, a classifier can be trained using training datasets for 1, 2, 3, 4, 5, 6, 7, 8, 9, 10, 11, 12, 13, 14, 15, 16, 17, 18, 19, 20, at least 1, at least 2, at least 3, at least 4, at least 5, at least 6, at least 7, at least 8, at least 9, at least 10, at least 20, at least 50, at least 100, at least 200, at least 300, at least 400, at least 500, at least 1,000, at least 2,000, at least 5,000, at least 10,000, at least 100,000, at least 1,000,000, from 1 to 10, from 10 to 20, from 20 to 50, from 50 to 100, from 100 to 500, from 500 to 1,000, from 1,000 to 10,000, or from 10,000 to 1,000,000 rounds.

Unsupervised classifiers can be useful in determining categories (e.g., defining case and control classes, or defining features of a response pattern) for analysis of a dataset when the quantity or identities of the categories have not been determined or provided to the classifier. In some cases, an unsupervised classifier can involve segregating datasets (e.g., segregating subjects or feature values of response patterns, such as biomarker values or cell parameter values) into groups or clusters based on similarities and/or differences in the datasets. Unsupervised classifiers can also offer the advantage of low requirements for computing power, which can be useful if the classifier is to be used on desktop computer systems that may lack extra computing power. Examples of supervised classifiers include support vector machines, linear regression models, logistic regression models, and multi-class classification models. Examples of unsupervised classifiers include k-means clustering models, principal component analysis models, and association rules models. A classifier useful in the systems and methods described herein can comprise one or more supervised classifier and/or one or more unsupervised classifier.

Semi-supervised classifiers can be useful in determining analyzing, parsing, integrating, or classifying datasets obtained using the systems, compositions, and/or methods described herein or provided from a medical history or other relevant assays or experiments. In some cases, semi-supervised classifiers can be trained using unlabeled data and labeled data, as described above in regard to supervised and unsupervised classifiers, and can result in a classifier with improved predictive accuracy compared to unsupervised classifiers and/or lower computational power requirements than supervised classifiers.

Classifiers useful in the systems and methods disclosed herein can include Naive Bayes classifier, support vector machines (SVM), k-nearest neighbor classifier, linear regression models, logistic regression models, relevance vector machines (RVM), decision tree classifiers. Classifiers useful in the systems and methods disclosed herein can be an ensemble classifier comprising one or more of the classifier types listed herein.

In some embodiments (e.g., wherein the factors in a sample biomarkers associated with lung cancer are known), one can use iCAP, or method of use or a kit thereof, to generate test response patterns that are compared directly against a response pattern of a sample positive for lung cancer and a response pattern of a sample negative for lung cancer and measuring known lung cancer biomarkers. A statistically significant similarity between a test response pattern and a positive response pattern as compared to the negative response pattern can suggest an increase risk or presence of lung cancer. Alternatively, one may compare the response patterns of a positive sample and a negative sample to generate a differential response pattern (which comprises biomarker(s) for lung cancer), which can then be used to evaluate the test response pattern to determine the test response pattern's similarity to a positive or negative response pattern, wherein a greater similarity to a positive response pattern as compared to the negative response pattern suggests an increased risk or presence of lung cancer.

In some embodiments, data from a cellular response assay (e.g., a set of one or more indicator cell biomarkers) as disclosed herein can be used in combination with one or more additional parameters related to the subject from which the test sample was obtained, such as medical history, gender, age, smoking history or status, co-morbidity, diagnostic imaging data, such as CT scans, size of a lesion or nodule, etc.

In some embodiments, a response pattern adapted for cellular response assay can comprise one or more response pattern features (cell parameters or biomarkers) that is detectable by an indicator cell, which can be any chemical or biological factor to which the indicator cell is responsive, and can result in a measurable change in the indicator cell. Different classifiers can be combined to increase the accuracy of a test. In some embodiments, a cellular response assay can comprise one, two, three, four, five, six, seven, eight, nine, ten, or more than two, more than five, or more than ten response pattern features.

In some embodiments, a response pattern can comprise at least 1, 2, 3, 4, 5, 6, 7, 8, 9, 10, 20, 30, 40, 50, 60, 70, 80, 90, 100, 200, 500, 1000, or more than 5, more than 10, more than 50, more than 100, or more than 500 response pattern features (e.g., biomarkers, elements or factors associated with lung cancer, such as, elements of a transcriptome, proteome, metabolome, or secretion profile of the responder cells, such as level of a protein, mRNA, RNA, DNA methylation, post-translational modification, cytokine secretion, or a metabolite). In some embodiments, one or more classifiers can be used to determine whether a test sample, e.g., a biological sample or fluid, from a subject comprises a benign or malignant nodule. In some embodiments, one or more classifiers can be used to determine the type of lung cancer, e.g., non-small cell lung cancer, adenocarcinoma, squamous cell carcinoma, and large cell carcinoma. In some embodiments, given a type of lung cancer, a classifier can be used to determine the progression stage (e.g., stage I, II, III, or IV) of a given lung cancer detected or identified using the systems, compositions, and methods described herein. In some embodiments, a classifier can be used to determine if a subject or sample thereof should be subjected to additional testing (e.g., biopsy). In some embodiments, a classifier can be used to identify a subject's risk for developing lung cancer, or to differentiate pre-invasive from invasive or metastatic lung cancer. In some embodiments, a classifier can be used to determine a treatment that is most efficacious or responsive to a subject's lung cancer. In some embodiments, one or more classifiers can be used as companion diagnostics to identify the subset of lung cancer patients who are most responsive to a specific therapy, e.g., chemotherapy or a combination therapy. In some embodiments, one or more classifiers can be used as a follow-up to a CT scan or to increase the accuracy of an imaging tool.

In some embodiments, a response pattern can comprise a set of genes belonging to a signaling pathway, used in the cellular response assay to provide information on an aspect of the biological sample. Response patterns can include, but may not be limited to, a set of biomarkers that can be used to distinguish a benign nodule from a malignant or cancerous nodule, or a nodule having a high risk of becoming cancerous; a set of biomarkers for distinguishing different stages of lung cancer; a set of biomarkers for distinguishing different types of lung cancer, e.g., non-small cell lung cancer, adenocarcinoma, squamous cell carcinoma, and large cell carcinoma. In some embodiments, a response pattern can comprise one biomarker, or a set, panel, or group of biomarkers. In some embodiments, indicator cells can be responsive to at least 2, 3, 4, 5, 6, 7, 8, 9, 10, or more than 2, more than 5, or more than 10 biomarkers. In some embodiments, indicator cells can be cultured, cloned, or engineered to be responsive to at least 2, 3, 4, 5, 6, 7, 8, 9, 10, or more than 10 biomarkers for lung cancer. In some embodiments, classifiers can be configured or designed to detect or diagnose different stages of lung cancer, e.g., stage 1, 2, 3, or 4.

A biomarker from a sample (e.g., a factor present in a sample from a subject, such as a lung cancer biomarker) can be a gene, DNA, RNA, cytokine, protein, immunoglobulin, cell receptor, or metabolite that is associated with lung cancer. Examples of lung cancer biomarkers can include, but may not be limited to, EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, or any combination thereof, one or more response pattern features of a trained iCAP lung cancer classifier (which can include ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, and/or ZNF318).

In some embodiments, a reporter gene or a marker, e.g., a gene encoding a fluorescent protein or an enzyme producing a luminescent or colored product, may be engineered into indicator cells to facilitate detection of response patterns, e.g., gene expression profile, of indicator cells.

Classifiers can use gene sets as parameters or features. Examples of gene sets tested include genes of the KEGG lung cancer pathways, lung cancer secretome gene sets, and several lung cancer-related gene expression gene sets. Normal large-airway epithelial cells can transcriptionally respond to aggressive lung cancer in vivo. In some embodiments of indicator cell assay classifiers, differential regulation of specific gene set(s) can be measured and evaluated instead of individual genes in order to reduce the number of interrogated cell parameters or to reduce the number of features informing the classifier and improve the classifier performance. In some embodiments, a robust differential expression between patient/case samples and control serum samples may be used while a specific gene set is not.

Systems and methods disclosed herein can comprise a computer having a processor and a non-transitory memory. The memory of computer of a system described herein can comprise instructions that, when executed, cause the computer to perform method steps as disclosed herein. For example, a computer of a system disclosed herein can comprise instructions and memory for storing and/or training one or more classifier or dataset disclosed herein. In some cases, a computer of a system or method disclosed herein can comprise a server and means for communication with other devices, such as one or more instrument used in the collection and/or analysis of samples or indicator cells, one or more remote user terminals, one or more database, and/or one or more remote processor (e.g., a remote processing cluster for performing data analysis or classifier training, validation, or analysis).

### Response Patterns

Response pattern can refer to the output, signal, or read-out of indicator cells. Response patterns can be grouped according to known attributes of one or more subjects (e.g., a known physiological condition or state) or according to the values measured or determined for the set of features of the response pattern.

In some cases, a response pattern feature (e.g., cell parameter or biomarker) can be a characteristic that can be measured and evaluated to indicate presence of normal or pathological process, pathological state, environmental exposure, outcome of disease or response to therapy. In some embodiments, a biomarker can be a substance or process in the plasma, or one or more features of the differential response pattern of indicator cells. An indicator cell can comprise a biomarker or a set of biomarkers. In some cases, the measurement and evaluation of one or more indicator cell biomarkers can be used to indicate the presence of a physiological state (e.g., a normal or pathological process, a pathological state such as lung cancer, an environmental stimulus, an outcome of a disease, or a response to therapy). A classifier can allow one to classify, diagnose, or differentiate a sample from a subject. For example, a classifier can be used to identify a disease state, stage of lung cancer, risk for lung cancer, type of lung cancer, or whether an indeterminate nodule is benign or malignant. In some embodiments, a classifier can comprise a computational model that has been trained (and, preferably, validated) for classifying a test sample using the cellular response assay described herein. In some embodiments, a classifier can be used to determine a differential response pattern, which can comprise a set of features, elements, or parameters. In some embodiments, the set of features, elements, or parameters of the differential response pattern (e.g., as determined by comparing response patterns produced in contacting a first indicator cell population with a first sample, such as a sample from a patient with unknown risk for lung cancer or a positive control sample, and a second sample, such as a positive or negative control sample), allow for the determination of the similarities and/or differences between a test response pattern (e.g., a response pattern determined by contacting an indicator cell population with a sample from a patient of unknown risk for lung cancer) and the response pattern generated when an indicator cell population is contacted by a sample known to have lung cancer.

A response pattern can comprise features (e.g., parameters of one or more indicator cell, such as a biomarker). In some cases, the features of a response pattern (e.g., a key response pattern) can be selected based on their individual utility in determining the physiological state or risk of a physiological state (e.g., lung cancer) in a subject from which an assayed sample is derived. The features of a response pattern can be selected to allow or improve the ability of a method or system disclosed herein to distinguish between a subject having lung cancer and one that is free of lung cancer or between a subject having a first risk of lung cancer and a subject having a second (e.g., known) risk of lung cancer. In some cases, the selection of features for use in a response pattern (e.g., a key response pattern) can be performed or aided, using a classifier. In some cases, a response pattern can comprise response pattern features that are indicative of a sample from a subject having lung cancer that make the classifier more specific to lung cancer. An iCAP system or method disclosed herein can be generated and tested using cross-validation techniques and then validated using independent test sets of data from new subjects. iCAP systems and methods of using iCAP systems can comprise a classifier and can be trained through an iterative process. Such classifiers can be based on features of a differential response pattern determined either from global expression response patterns of indicator cells to samples, or based on targeted response pattern of a subset of biomarkers known or predicted to be specific for lung cancer. In some embodiments, a differential response pattern can comprise quantitative or qualitative changes in levels of iCAP biomarkers between affected and unaffected samples.

In some cases, a response pattern can comprise features (e.g., indicator cell parameters measurable or detectable in an indicator cell population, such as a gene expression level or protein concentration) other than the measurable or detectable factors present in a sample obtained from a subject.

In addition to the features of a response pattern (e.g., a response pattern determined by a cellular response pattern or a differential response pattern), the presence or progression of a physiological condition, such as lung cancer, in a subject can be determined based partially or fully on biographical information and/or additional medical or experimental data. For example, one or more features used to determine the presence or progression of a physiological condition in a subject can comprise biographical information (e.g., one or more of: gender, height, weight, family medical history, cancer history, impaired lung function, history of exposure to environmental or occupational toxins or ionizing radiation (e.g., asbestos, radon, or uranium), genetic predisposition, low consumption of fruits and vegetables, and/or history of smoking or smokeless tobacco use) and/or additional medical or experimental data (e.g., results from one or more additional tests or experiments, such as MRI results, CT scan results, X-ray results, stress test results, traditional clinical blood tests, or biopsies). In some embodiments, one or more features comprising biographical information and/or additional medical or experimental data can be used to train a classifier or to generate a differential response pattern, as described herein. In some cases, one or more features comprising biographical information and/or additional medical or experimental data can be used to train a classifier or to generate a differential response pattern in combination with all or a portion of the features comprising a response pattern determined using a cellular response assay.

In some embodiments, a response pattern can include a panel of indicator cell parameters (e.g., features) known to be associated with lung cancer. In some cases, the presence of lung cancer or a risk of lung cancer in a subject can be indicated by or can be detected from a measured or detected value one or more indicator cell parameters (e.g., EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, or any combination thereof) in a sample by differentially expressing one or more biomarkers (e.g., indicator cell parameters). For example, an indicator cell contacted by a sample may be used to obtain a response pattern comprising data (e.g., response pattern feature values) comprising levels (e.g., gene expression levels of one or more of genes selected from ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, or ZNF318 (including, e.g., gene expression data, protein expression data, protein activity data, and/or nucleic acid transcription data). In some embodiments, classifiers for lung cancer can comprise different combinations of response pattern features indicative of or correlated with the presence of, the absence of, or a risk for lung cancer. In some embodiments, a classifier can comprise a panel of elements/factors (e.g., features) in the differential response pattern based on comparison of response patterns of a sample positive for lung cancer and a sample negative for lung cancer wherein the panel of elements/factors in the differential response pattern may not be identified or known previously to be associated with lung cancer (for example, some iCAP systems and methods described herein include features that are strongly predictive of the presence of lung cancer in a subject, which have not previously been shown to be associated with cancer, including CACNG6, HAGHL, IFNL2, KIRREL2, CTF1, ARMCX4, and IFNK). Such classifiers can comprise one or more elements of a transcriptome, proteome, metabolome, or secretion profile of the indicator cells (e.g., protein, mRNA, RNA, DNA modification, DNA methylation, cytokine, or cellular byproduct). Such elements or factors can be evaluated individually or in combination. In some cases, 5 or more, 10 or more, 15 or more, 20 or more, 30 or more, 40 or more, 50 or more, 60 or more, 70 or more, 80 or more, 90 or more, 100 or more elements or factors (e.g., features) can be identified in a differential response pattern and/or used to evaluated response patterns of test samples by indicator cells.

In some cases, a response pattern useful in the methods and system described herein can comprise a plurality of response pattern features. In some cases, a set of response pattern features (and measured values thereof) useful in the methods and systems described herein can comprise data (e.g., response pattern feature values). In some cases, a response pattern can comprise values (e.g., gene expression levels) of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more than 35 of the genes selected from: EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNERSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some cases, the accuracy of a method or system described herein can be increased if one or more response patterns comprises response pattern feature values (e.g., gene expression levels) of at least 20 genes selected from: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some cases, the accuracy of a method or system described herein can be increased if one or more response patterns comprises response pattern feature values (e.g., gene expression levels) of each of the following genes: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some aspects, the accuracy of an iCAP system can be improved when the one or more response pattern feature values used in an iCAP system comprise an expression level of each of the following genes: CACNG6, PRKCA, ROR2, RSBN1, PDZD7, CCDC66, ANKRD37, HAGHL, MT-ND4, BMP6, RASAL1, CEMIP, SPOCD1, PRR22, IFNL2, TRIM2, KIRREL2, CTF1, ARMCX4, and IFNK. In some aspects, the accuracy of an iCAP system can be improved when the one or more response pattern feature values used in an iCAP system comprise an expression level of each of the following genes: CACNG6, PRKCA, ROR2, RSBN1, PDZD7, CCDC66, ANKRD37, HAGHL, MT-ND4, BMP6, RASAL1, CEMIP, SPOCD1, PRR22, IFNL2, TRIM2, KIRREL2, CTF1, ARMCX4, and IFNK.

In some cases, a response pattern of an iCAP method or system indicating the presence of a physiological state of interest (e.g., lung cancer) or an increased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an increase in the value of the feature (e.g., measured in an indicator cell population), for example, as compared to corresponding response pattern feature value(s) measured (e.g., in a separate indicator cell population) using a negative control sample.

In some cases, a response pattern of an iCAP method or system indicating the presence of a physiological state of interest (e.g., lung cancer) or an increased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an increase in the value of the feature (e.g., measured in an indicator cell population), for example, as compared to corresponding response pattern feature value(s) measured (e.g., in a separate indicator cell population) using a positive control sample.

In some cases, a response pattern of an iCAP method or system indicating the presence of a physiological state of interest (e.g., lung cancer) or an increased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an decrease in the value of the feature (e.g., measured in an indicator cell population), for example, as compared to corresponding response pattern feature value(s) measured (e.g., in a separate indicator cell population) using a negative control sample.

In some cases, a response pattern of an iCAP method or system indicating the presence of a physiological state of interest (e.g., lung cancer) or an increased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an decrease in the value of the feature (e.g., measured in an indicator cell population), for example, as compared to corresponding response pattern feature value(s) measured (e.g., in a separate indicator cell population) using a positive control sample.

In some cases, a response pattern of an iCAP method or system indicating the presence of a physiological state of interest (e.g., lung cancer) or an increased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing a lack of change (or lack of significant change) in the value of the feature (e.g., measured in an indicator cell population), for example, as compared to corresponding response pattern feature value(s) measured (e.g., in a separate indicator cell population) using a negative control sample.

In some cases, a response pattern of an iCAP method or system indicating the presence of a physiological state of interest (e.g., lung cancer) or an increased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing a lack of change (or lack of significant change) in the value of the feature (e.g., measured in an indicator cell population), for example, as compared to corresponding response pattern feature value(s) measured (e.g., in a separate indicator cell population) using a positive control sample.

In some cases, a response pattern of an iCAP method or system indicating the absence of a physiological state of interest (e.g., lung cancer) or a decreased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an increase in the value of the feature (e.g., measured in an indicator cell population), for example, as compared to corresponding response pattern feature value(s) measured (e.g., in a separate indicator cell population) using a negative control sample.

In some cases, a response pattern of an iCAP method or system indicating the absence of a physiological state of interest (e.g., lung cancer) or a decreased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an increase in the value of the feature (e.g., measured in an indicator cell population), for example, as compared to corresponding response pattern feature value(s) measured (e.g., in a separate indicator cell population) using a positive control sample.

In some cases, a response pattern of an iCAP method or system indicating the absence of a physiological state of interest (e.g., lung cancer) or a decreased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an decrease in the value of the feature (e.g., measured in an indicator cell population), for example, as compared to corresponding response pattern feature value(s) measured (e.g., in a separate indicator cell population) using a negative control sample.

In some cases, a response pattern of an iCAP method or system indicating the absence of a physiological state of interest (e.g., lung cancer) or a decreased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing an decrease in the value of the feature (e.g., measured in an indicator cell population), for example, as compared to corresponding response pattern feature value(s) measured (e.g., in a separate indicator cell population) using a positive control sample.

In some cases, a response pattern of an iCAP method or system indicating the absence of a physiological state of interest (e.g., lung cancer) or a decreased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing a lack of change (or lack of significant change) in the value of the feature (e.g., measured in an indicator cell population), for example, as compared to corresponding response pattern feature value(s) measured (e.g., in a separate indicator cell population) using a negative control sample.

In some cases, a response pattern of an iCAP method or system indicating the absence of a physiological state of interest (e.g., lung cancer) or a decreased risk of a physiological state of interest (e.g., lung cancer) can comprise 0, 1, 2, 3, 4, 5, 6, 7, 8, 9, 10, 11, 12, 13, 14, 15, 16, 17, 18, 19, 20, 20 to 25, 25 to 30, 30 to 40, 40 to 50, or greater than 50 features representing a lack of change (or lack of significant change) in the value of the feature (e.g., measured in an indicator cell population), for example, as compared to corresponding response pattern feature value(s) measured (e.g., in a separate indicator cell population) using a positive control sample.

In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent an decrease in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample). In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent an increase in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent a decrease in the value of the feature in the second response pattern. In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a lack of change (or lack of significant change) in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent a decrease in the value of the feature in the second response pattern.

In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent an increase in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample). In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a decrease in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent an increase in the value of the feature in the second response pattern. In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a lack of change (or lack of significant change) in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent an increase in the value of the feature in the second response pattern.

In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a lack of change (or lack of significant change) in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample). In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent an increase in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent a lack of change (or lack of significant change) in the value of the feature in the second response pattern. In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a decrease in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent a lack of change (or lack of significant change) in the value of the feature in the second response pattern.

In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a decrease in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent an increase in the value of the feature in the second response pattern, while a portion (e.g., 1 to 10) of the response pattern feature values of the first response pattern represent a lack of change (or lack of significant change) in the value of the feature in the second response pattern. In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent an increase in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent a decrease in the value of the feature in the second response pattern, while a portion (e.g., 1 to 10) of the response pattern feature values of the first response pattern represent a lack of change (or lack of significant change) in the value of the feature in the second response pattern. In some cases, a portion (e.g., 1 to 5, 5 to 10, 10 to 15, 15 to 20, 20 to 30, 30 to 50, or greater than 50) of the response pattern feature values of a first response pattern of an iCAP method or system can represent a lack of change (or lack of significant change) in the value of the feature (e.g., measured in an indicator cell population), for example, versus corresponding response pattern feature value(s) of a second response pattern of the iCAP method or system (e.g., a response pattern comprising response pattern feature values measured from indicator cells contacted with a positive or negative control sample) while a portion (e.g., 1 to 10) of the response pattern feature values of the first response pattern represent an increase in the value of the feature in the second response pattern, while a portion (e.g., 1 to 10) of the response pattern feature values of the first response pattern represent a decrease in the value of the feature in the second response pattern.

In some cases, a response pattern feature can comprise levels (e.g., measured or determined values) or changes in levels of a transcription factor. For example, a response pattern feature value can comprise an expression level of a transcription factor (e.g., as measured, detected, or determined using an iCAP system or method). In some cases, incorporating measured or determined values of transcription factors into a response pattern can improve the accuracy of methods or systems disclosed herein. For example, transcription factors can influence the types and quantities of proteins expressed by a cell. In some cases, an expression level of a transcription factor can be used to determine a risk of lung cancer in a subject. In some cases, a level of hypoxia in a subject or tissue of a subject can affect the composition of a sample of a subject. An expression level or change in expression of HIF1-alpha can be used (e.g., along with other response pattern features and response pattern feature values) to improve the determination of a risk of lung cancer in a subject, using an iCAP system or method.

In some cases, a response pattern does not need to reflect the underlying biology of disease progression in order to be used as a classifier of disease state, but can reflect the underlying disease if the response pattern comprises disease-specific cell parameters (e.g., response pattern features). For example, genes (e.g., measured or detected values thereof, such as a gene expression level) such as EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, or any combination thereof, genes involved in lung cancer-related cellular processes such as cell proliferation or hypoxia, one or more response pattern features of a trained iCAP lung cancer classifier, including ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318 can be present and/or enriched in the response pattern.

In some cases, a differential response pattern need not reflect the underlying biology of disease progression in order to be used as a biomarker or response pattern feature indicating a disease state. In some embodiments, overrepresentation (e.g., increased measured or detected values relative to a control) of disease-relevant genes, such as lung cancer biomarkers, in the signature response pattern can indicate active disease-relevant pathways in the indicator cells. In some embodiments, the presence of known signal transduction pathways or receptors in the indicator cell response can indicate specific biomarkers in the blood to which the cells are responsive. For example, measured or detected activity of one or more genes known to participate in signal transduction pathways or receptor pathways in the indicator cell population response pattern can indicate the presence or absence of lung cancer in a subject from whom a sample was taken and used to contact the indicator cell population. In some embodiments, one way to increase the signal-to-noise ratio of a classifier can be increased by including lung cancer specific biomarkers in a response pattern feature set of the classifier. In some embodiments, the set of key response pattern features (e.g., the signature response pattern) is the differential response pattern generated by comparing a response pattern of a sample positive for lung cancer and a response pattern of a sample negative for lung cancer. In some embodiments, two or more differential response patterns (e.g., differential response patterns from different indicator cell types) are combined to generate a composite set of key response pattern features (e.g., a composite signature response pattern), comprising a plurality of elements or factors that allow one to determine classify or diagnose a test subject.

In some embodiments, response pattern refers to expression pattern or profile of RNA, DNA, protein, metabolite, cytokine, miRNA, cellular co-factor, cell receptor, or any combination thereof. In some embodiments, response pattern refers to gene expression, transcriptome, proteome, metabolome, and/or secretion profile. In some embodiments, the expression pattern is gene expression. In some embodiments, response or expression pattern is measured by RNA-seq, PCR, direct measurement of RNA by digital optical bar codes, next-generation sequencing, reporter gene assay, or microarray. In some embodiments, a response patterns comprises the transcriptome, and/or proteome and/or the secretion profile (e.g., secretome), and/or metabolome, and/or lipidome of said cells.

In some embodiments (e.g., in some methods or systems for determining the presence of lung cancer and/or risk of lung cancer in a test sample), the response pattern generated from applying a test sample with a plurality of indicator cells may be compared to a negative control and/or a positive control. In some embodiments, a negative control can refer to a response pattern generated from applying a sample obtained from a healthy individual, or a sample without any lung cancer, to a plurality of indicator cells. In some embodiments, a negative control can comprise a sample obtained from a benign nodule, or a tissue that is not cancerous.

In some cases, a positive control can refer to a response pattern generated from applying a sample with a known risk of developing lung cancer (e.g., non-small cell lung cancer, adenocarcinoma, squamous cell carcinoma, and large cell carcinoma), or a sample with a known stage of a lung cancer, or a sample from a previously identified lung cancer tissue, to a plurality of indicator cells. In some embodiments, a positive control can comprise a sample from a malignant nodule. In some embodiments, a positive control comprises a sample from a subject who was previously diagnosed with a lung cancer. In some embodiments, a positive control can comprise a sample from a subject with a positive diagnosis for a lung cancer and is known to be responsive to a lung cancer therapy, such that the cellular response assay can be used to identify other patients who are likely to be responsive to the lung cancer therapy.

By comparing the response pattern from a test sample (e.g., a test response pattern), or a sample that needs classification or identification using the cellular response assay disclosed herein, with that of a negative and/or positive control, a differential response pattern, or a difference between the response patterns (e.g., between a control and the test response pattern), can be analyzed to determine how closely a test sample resembles a control, e.g., a negative control or a positive control, which can then be used to assess a subject's risk for lung cancer, stage of cancer, etc.

In various embodiments, the differential response pattern or any difference or alteration in response pattern analyzed using the cellular response assay may be statistically significant. In some cases, statistical methods known in the art can be used to determine the error bars, statistical significance, and the confidence intervals of the resultant response patterns. In some embodiments, samples can be tested in duplicates or triplicates to verify the results. In some embodiments, results of repeated assays can be averaged.

In some embodiments, a threshold is used to determine if the test sample is more like the negative control or the positive control, or used to assign different levels of lung cancer risk for the test sample. In some embodiments, a threshold for determining a response pattern from a test sample is similar to that of a negative and/or positive control is based on an overlap in their response patterns, wherein the overlap is at least 25%, 30%, 35%, 45%, 50%, 55%, 60%, 65%, 70%, 75%, 80%, 85%, 90%, 91%, 92%, 93%, 94%, 95%, 96%, 97%, 98%, 99%, or 100%. In some embodiments, the threshold refers to an overlap between a test sample response pattern and a control that is 30-40%, 35-45%, 40-50%, 45-55%, 50-60%, 55-65%, 60-70%, 65-75%, 70-80%, 75-85%, 80-90%, 85-95%, or 90-100%. In some embodiments, the threshold refers to an overlap between a test sample response pattern and a control that is 30-50%, 40-60%, 50-70%, 60-80%, or 70-90%. In some embodiments, the threshold refers to an overlap of at least 2, 3, 4, 5, 6, 7, 8, 9, 10, 11, 12, 13, 14, 15, 16, 17, 18, 19, or 20; or >2, >3, >4, >5, >6, >7, >8, >9, >10, >11, >12, >13, >14, >15, >20, >30, >40, >50 response features (e.g., measured cell parameters). For example, a response pattern or a set of key response pattern features indicative of a presence of malignant nodule comprises 10 response pattern features, wherein overlap or confirmation of any 5, 6, 7, 8, or 9 of such response pattern features in a test subject is assigned a 60%, 70%, 75%, 80%, or 85% chance, respectively, of developing a malignant nodule. In some embodiments, the threshold is validated by refining the set of response pattern features (e.g., cell parameters) selected for a response pattern (e.g., through the analysis of indicator response pattern feature values (e.g., cell parameter values) with a classifier).

In some embodiments, a measured or detected alteration or a change in a differential response pattern feature value that reflects a statistically significant difference (e.g., between a first response pattern and a second response pattern) is evaluated to classify a biological sample. In some embodiments, expression pattern differences or differential response patterns can be obtained by comparing response patterns of a plurality of indicator cells having been contacted with a test biological sample or a sample of unknown identity or risk for lung cancer with the same indicator cells having been contacted with a control sample, such as a biological sample from a healthy or cancer-free subject or a sample with a known risk of lung cancer. By comparing response patterns between pluralities of indicator cells exposed to different samples (e.g., negative control or positive control), one can detect and/or measure the differences in their response patterns to classify the test sample, or to ascertain the lung cancer risk of a test sample.

In some embodiments, a differential response pattern can be established by comparing response patterns obtained from fluids or biological samples from abnormal subjects, e.g., those diagnosed with lung cancer, with those obtained from fluids of normal subjects. The response patterns can be compared by identifying individual transcripts that are significantly differentially expressed between the two responses, such as EGFR, ALK, MET, ROS-1, KRAS, C-KIT, WASH7P, BRAF (V600E), HER2 (ERBB2), JAK2, PD-1, pro-gastrin-releasing peptide, carcinoembryonic antigen (CEA), neuron-specific enolase (NSE), cytokeratin 19 (CYFRA-21-1), alpha-fetoprotein, carbohydrate antigen-125 (CA-125), carbohydrate antigen-19.9 (CA-19.9), ferritin, CRP, HGF, NY-ESO-1, prolactin, or any combination thereof, one or more of ABL2, ADGRG1, ADRA1B, AKT3, ALPK3, ANKRD22, ANKRD37, ARMCX4, CACNG6, CCDC66, CEMIP, CTF1, DEPP1, FAXDC2, FBXL5, GPR17, HAGHL, HIF1A, IFNL2, IFNK, IL1R2, KIRREL2, LOXL2, MT-ND4, NEDD9, PDZD7, PRKCA, PRR22, PWP2, RASAL1, RNF223, ROR2, RSBN1, SLC2A3, TRIM2, ANPEP, ARSA, C2ORF69, CALD1, CBX1, CLIP4, COL6A1, COQ4, DDAH1, DLG1, DUSP6, EPHB6, FAM72A, FGF1, FLIP1L GJA5, GPR143, IL18, LAMA1, LEPR, LRRN4, MMP9, MTMR10, MT1F, MT1M, MT1X, NSRP1, PLK2, PSG5, S1PR1, SFTA1P, SLC39A10, STX3, SUSD2, SYNPO2, TCF25, TGFB2, TM4SF1, TRIM65, TSKU, TXNRD1, UBE2J1, WAC, WDR13, MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, VEGFA, CDH1, IGFBP3, BRAT1, FAM174B, PRDM1, STS, USP53, PEAR1, DMBT1, NPR1, BNIP3L, BHLHE40, MID1, CCNG2, KDM3A, TMEM154, NOG, KCP, KISS1, PRSS22, HLA.V, AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318, or by generating and testing a more complex disease classification model using mathematical or machine-learning approaches, support vector machines, or random forest algorithms. In some embodiments, analysis can be expanded to obtain a longitudinal or cross sectional set of disease signatures, by obtaining complex multicomponent readouts from indicator cells (e.g., gene expression microarrays) after exposure to samples obtained from normal or diseased subjects taken at various stages of disease progression. In longitudinal studies, a single subject at various disease stages may be assessed, whereas in cross-sectional diagnoses, multiple subjects at various disease stages can be used as subjects.

In some embodiments, an indicator cell assay platform (iCAP) system or method can overcome barriers such as low abundance of disease marker molecules, high levels of noise, and the potential diagnostic complexity of disease by circumventing the need to directly identify molecules in blood, and instead capitalizing on the natural ability of cells to detect and respond to disease signatures in blood. An iCAP assay can involve exposing standardized, cultured cells to serum from diseased and normal patients, identifying a global differential transcriptional response of the cells to the serum (e.g. using RNA sequencing (RNA-seq)), and using disease classification tools to identify a subset of features that can reliably classify disease state. In some cases, instead of using a global approach to identify the differential transcriptional response of cells to serum, differential response is measured from only a subset of features known or predicted to be related to the disease or condition.

In some cases, deploying the assay can involve analyzing gene expression changes that inform the classifier using cost-effective approaches known in the field, e.g., microarray, next generation sequencing, PCR, Taqman® or Nanostring® technology.

In some embodiments, a lung cancer iCAP system or method can comprise performing a blood test (e.g., obtaining a blood sample) for patients with IPNs (for example IPNs with a diameter of 3-25 mm) identified by chest CT, to identify those with benign nodules without the need for invasive biopsy, while focusing further diagnostic tests on those with higher risk of lung cancer. iCAP can be applied at the time of identifying a suspicious nodule by CT that will give patients a probability of disease using a continuous variable. An iCAP system or method can comprise a visual representation of the data which convey to patients a lung cancer risk and may allow patients to choose the best course of action.

Interactions between indicator cells and a test sample (or a test substrate, such as a blood biomarker for lung cancer) can be used to produce a set of key response pattern features (e.g., a signature) indicative of a disease or cancer, such as lung cancer. In some cases, such key response patterns (e.g., indicator cell response signatures, fingerprints, or profiles, which can comprise the set of key response pattern features) can be produced by evaluating the response pattern(s) (e.g., values of the features of a first response pattern) obtained when one or more control samples (e.g., a positive control sample and/or a negative control sample) brought into contact with one or more respective indicator cell populations can be used to determine whether a sample is positive or negative for having or developing a disease or cancer, such as lung cancer.

Changes in measurable or detectable indicator cell parameters (e.g., values of one or more features of the response pattern or signature response patterns) can result when an indicator cell interacts with one or more components or factors present in a biological fluid or fraction thereof, or a sample that corresponds to a particular disease, condition, or stage of progression of a disease or condition. These parameters can be referred to as “indicators” in some cases, e.g., because they can be used in part as indicators of a disease, condition, or stage of a physiological condition or state. In some cases, their identity need not be apparent from the resultant pattern or understood for indicator cells to detect or present with a differential response pattern relative to a control sample. In some cases, a strength of the iCAP system is that individual biomarkers, or response pattern features, do not need to be known in advance. In some cases, a set of biomarkers present in cancerous or pre-cancerous lung tissues of a subject may not be identical to a set of response pattern features, key response pattern features, or differential response pattern features used or determined using an iCAP system or method. In some embodiments, a response pattern can also comprise detectable markers or dyes, such as exogenously incorporated dyes or fluorescent tags (e.g., through exogenously introduced plasmids or nucleic acids).

In some cases, indicator cells can interact with a plurality of factors in a sample to produce a signature or response pattern. The number of features (e.g., parameters, elements or signals) in the response pattern or profile can be at least 3 or more, or at least 5, at least 10, at least 15, at least 20, at least 25, at least 30, at least 35, at least 40, at least 45, or at least 50. In some embodiments, indicator cells can interact with more than 5, 10, 20, 30, 40, or 50 factors in a sample. The number of features of a response pattern (e.g., parameters, elements, or signals) may be 3 to 50 or more than 50, including all integer numbers between 3 and 50.

In some embodiments, more than one type of indicator cell can be used to create a response pattern. In some cases, more than one type of indicator cells may be maintained or cultured in the same culture or as separate cultures. In some cases, at least 1, at least 2, at least 3 at least 4, at least 5, at least 6, at least 7, at least 8, at least 9, or at least 10 types of indicator cells can be used in an iCAP assay, either together, in tandem, or sequentially.

Some indicator cells may be of the type related to the disease or condition, while other cell types may be unrelated. In some embodiments, one or more indicator cells can be used to validate a result or response pattern.

More complex measurements can also be obtained by measuring components of cellular regulation, such as protein synthesis, RNA, microRNA, and variations in RNA splicing. Gene expression profiles can be obtained using one or more microarray, sequencing, and/or immunoprecipitation methods. In some embodiments, one or more gene expression detection methods can be used, including, but not limited to, PCR, RNA-seq, direct detection with digital barcodes or next-generation sequencing methods.

In some embodiments, the parameters or indicators (e.g., factors in a sample, indicator cell parameters and/or data from additional assay(s) or biographical or medical background) that result in the profile or response pattern can include, but are not limited to, biomarkers or factors known to characterize or be associated with the disease or cancer, such as lung cancer. In other cases, measured parameters (e.g., indicator cell parameters measured in an iCAP system or method) may not require the sample biomarkers or disease indicators to be known. In some embodiments, the utility of the iCAP methods does not require knowledge or understanding of the factors (e.g., biomarkers from a sample) that are measured or detected by indicator cells, which allows for broader application of iCAP than assays based on specific biomarkers.

In one aspect of the iCAP methods and systems, biological fluid from a subject or subjects with a known abnormal condition may be used to establish a baseline pattern in indicator cells. In some cases, it is not necessary to know in what manner the components of the biological fluid are altered by the abnormal condition or disease as the differential response pattern obtained from the indicator cells can be used to diagnose a disease or cancer, such as lung cancer.

In some embodiments, the response pattern of indicator cells in the abnormal condition can be compared with the response pattern of indicator cells contacted with normal biological fluid or a control. The differential pattern exhibited by the indicator cells can be used for comparing to the response patterns from test samples. In some embodiments, a differential pattern can be established by identifying elements of a response pattern exhibited as a result of contact with test samples representing an abnormal condition or disease from elements in patterns established by a control or a normal sample.

In some cases, the detection rate or accuracy of the iCAP assay can be enhanced by excluding elements or factors that do not vary between the normal/control sample and test samples. Excluding such non-varying parameters that do not contribute to the differential response patterns or are not indicative of a disease can increase the signal over background noise and improve the performance of the iCAP assay. In some aspects, the detection rate or accuracy of the iCAP assay can be enhanced by excluding elements or factors in the differential response pattern that provide redundant information to other elements or factors in the differential response pattern.

In some embodiments, the response patterns of parameters obtained from a subject with known abnormal conditions or stage of disease with that of a test subject can be compared directly. A strong correlation or similarity between such test response pattern against the response pattern of a positive control can be used to determine the subject's risk of developing or having the abnormal condition, disease, or cancer.

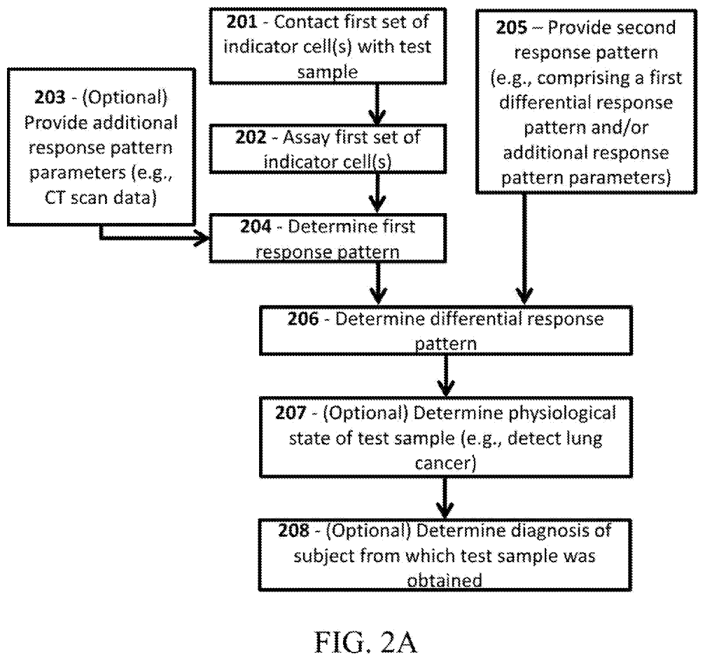

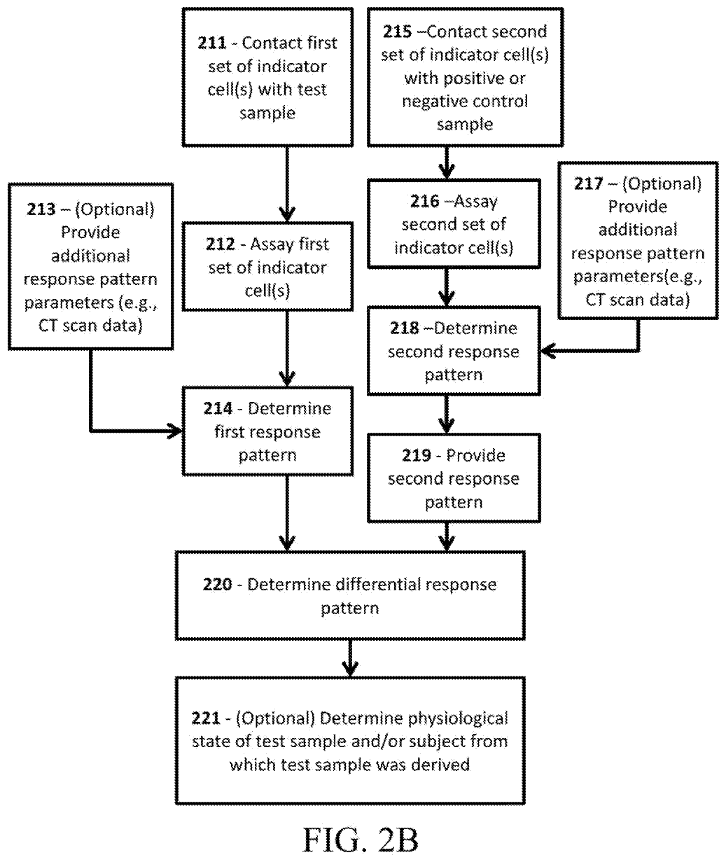

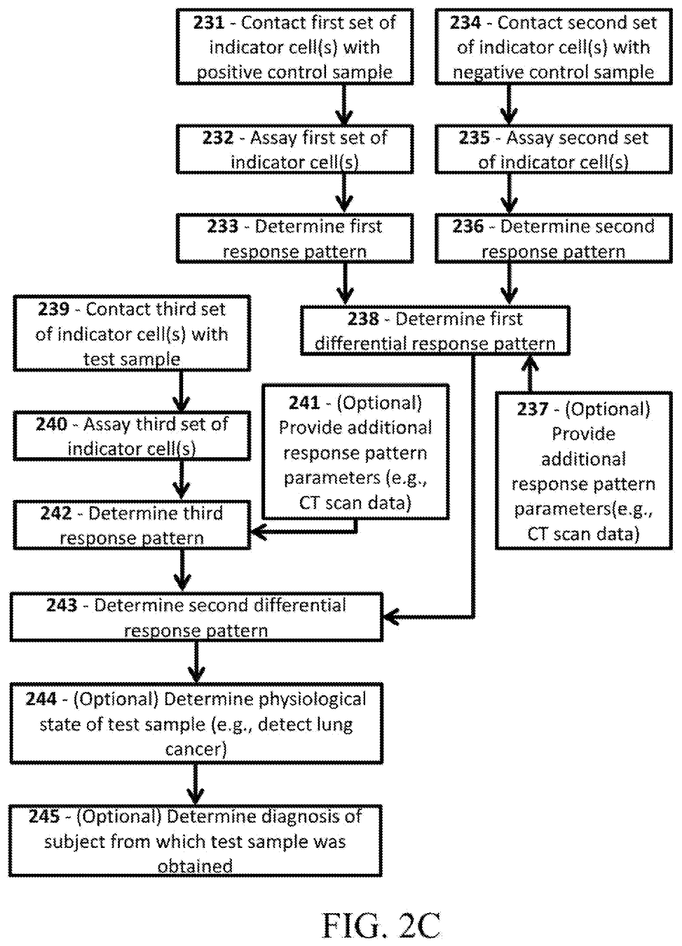

A “differential pattern” or “differential response pattern” as used herein can refer to a response pattern obtained by comparing the response pattern generated by indicator cells in contact with a sample from a subject with a known condition or stage of condition (e.g., a positive response pattern) with a response pattern generated by indicator cells in contact with a sample from a subject known to be negative for a disease/condition (negative response pattern). In some cases, a differential response pattern can be determined from a first response pattern determined by contacting a first set of indicator cells with a sample from a subject with an unknown physiological state and/or unknown risk for a physiological condition (e.g., lung cancer) and one or more additional response patterns (e.g., comprising one or more differential response patterns) determined by contacting a second set of indicator cells with a sample (e.g., test sample) from a subject with a known physiological state (e.g., positive or negative) or risk for a physiological condition (e.g., as shown in FIG. 2A and FIG. 2B). In some cases, the second response pattern can be a differential response pattern. FIG. 2C shows an example of a method comprising the determination of a differential response pattern from a first response pattern determined from a positive control sample and a second response pattern determined from a negative control sample. In some cases, a differential response pattern (e.g., a second differential response pattern) can be determined from a response pattern (e.g., a first differential response pattern) and a third response pattern (e.g., a response pattern determined from a sample from a subject with unknown physiological state or risk of a physiological state (e.g., risk of lung cancer), for example, as shown in FIG. 2C. A differential pattern can also be generated by comparing a response pattern generated by indicator cells in contact with a sample from a test subject (e.g., a subject with an unknown risk for lung cancer) with either the positive or negative response pattern. The negative response pattern can also be generated by indicator cells in contact with a buffer or indicator cells that have not been contacted with any extraneous biological fluid/sample (negative response pattern). Each pattern can be normalized to a control. The normalization factor can be an internal control derived from the response pattern itself, such as the average expression level of a group of genes that are known to be stable or unresponsive across a variety of conditions. The normalization factor can be an external control such as a second normalizing pattern obtained when the indicator cells are contacted with fluids or fractions from one or more normal tissue or disease-free subjects, which can be used to determine the background signal. Other types of normalizing patterns could be used, including, but not limited to, a pattern obtained when the cells are cultured in the absence of any biological fluids other than culture media. In both cases, the differences can be evaluated statistically depending on the number of subjects included in any of these groups. Thus, if sufficient numbers of independent patterns are used, statistically significant differences can be evaluated, and if desired, can be used as a criterion for including a specific parameter in the final response pattern or profile. In some cases, only a single subject is used to create a standard for abnormal condition or disease stage and a single normal control may be used. In some embodiments, multiple response patterns can be independently generated from a sample in order to generate an average response pattern to minimize fluctuations in the sample or in detecting response patterns.

In some embodiments, one can identify individual components of a biological fluid that effect complex changes in indicator cells or result in differential response patterns. In some embodiments, iCAP assays provide an approach to identify a risk for a disease or physiological state from body fluids collected from a subject. For example, analyzing the differential expression profile after exposure to a positive control sample (e.g., a positive control serum) known to have diseased or lung cancer cell elements and negative control serum that does not have the disease can lead to the discovery of biological pathways in the indicator cells that are activated or repressed by exposure to the disease serum. These targeted biological pathways can include cell surface receptors with known substrates, which indicate the substrate as a blood biomarker for lung cancer.

Elements and signals of a response pattern can be defined or validated in a number of ways, including sequencing of nucleic acids (e.g., DNA, RNA, or mRNA), identification of proteins/peptides, microarray, digital barcode technology, direct detection (e.g., mass spectrometry or sequencing), indirect detection, light microscopy, reporter assays, and cell morphology analyses. Indirect detection can comprise detection of detectable markers or reporters associated with a protein or nucleic acid, such as an antibody, an aptamer, or a fusion protein tagged with a detectable marker, which can comprise a fluorophore, chemiluminescence, or a radionuclide. Indirect detection can also include detection of enzymatic activity of a protein or evidence of specific enzymatic activity on a molecule of interest (e.g., an element or signal comprising a component of the response pattern).

Indirect detection can include immunohistochemistry, immunoprecipitation, oligonucleotide hybridization, microarrays, polymerase chain reaction (PCR), reverse-transcription PCR (rt-PCR), fluorescence in situ hybridization (FISH) or Western blotting. Sequencing of elements or signals that comprise components of the response pattern can include DNA-seq, which can include Sanger sequencing and next-generation sequencing techniques, and RNA-seq.

iCAP systems and methods can comprise steps or components for assessing the expression levels of hundreds or thousands of genes, typically as a transcriptome, e.g., levels of mRNA or micro RNA present in the cell. iCAP systems and methods can also comprise steps or components for measuring the proteome, e.g., levels of multiple proteins that are produced in the cell. Methods of assessing gene expression can comprise direct or indirect measurement of mRNA present in a cell or fluid. The iCAP can have a multi-component gene expression readout from a genetically identical population of cells, eliminating challenges due to variable abundances of particular cell types in blood, genetic variation between individuals and prominent responsiveness of immune cells to generic inflammatory signals. Gene expression analysis can also comprise the use of plasmids that include expression cassettes that can produce a detectable marker and that can be activated or inhibited by the presence of specific nucleic acids or oligonucleotides. iCAP systems and methods can comprise methods to interrogate secretion profiles, wherein cells may secrete multiplicities of materials into the environment. Other parameters that may be measured include the levels of various small molecules in the cells, e.g., the metabolome. In some embodiments, criteria can include behaviors of the cells themselves such as proliferation, changes in morphology, and the like.

### Biological Samples

Normal cells that grow in close proximity to cancer cells can be collected from subjects and their expression profiles can be used as indicators of cancer. Of the ongoing efforts to identify diagnostic and prognostic lung cancer biomarkers, breath- or blood-based assays have the advantage of being low-cost and non-invasive. Using blood-based assays, such as cellular response assay of blood or biological fluids, e.g., in combination with imaging diagnostic tools can boost the accuracy and sensitivity of diagnosis.

Test substrates or test samples (e.g., samples from patients having an unknown status with respect to a physiological state of interest, including a risk for having the physiological state) to be screened using an iCAP system or method described herein can include various biological fluids obtained from a subject or human patient, such as blood serum, blood plasma, urine, tissue sample, biopsy sample, or cell extract. A control sample can be obtained from an animal (e.g., an inbred, outbred, or engineered animal model), cell lines, human tissue banks, or human subjects. A test sample refers to a sample having at least one unknown physiological state (e.g., risk for lung cancer).

A sample can comprise one or more factor. A test sample can comprise a plurality of factors. In some cases, a test sample comprises at least 20 different factors, at least 50 different factors, at least 100 different factors, at least 1000 different factors, at least 10,000 different factors, at least 100,000 different factors, or at least 1,000,000 different factors. In some cases, 90% or less, 80% or less, 70% or less, 60% or less, 50% or less, 40% or less, 30% or less, 20% or less, 10% or less, 5% or less 1% or less, 0.1% or less, 0.01% or less, or 0.001% or less of the factors in a sample are detected, identified, evaluated, or analyzed. Representative examples of a factor of sample include a peptide, a polypeptide or fragment thereof (e.g., a protein), a nucleotide, a polynucleotide or fragment thereof (e.g., a nucleic acid, such as mRNA, tRNA, miRNA, rRNA, snRNA, snoRNA, gRNA, shRNA, siRNA, crRNA, tracrRNA, RNAi, genomic DNA, cell-free DNA, or a fragment of any thereof), a small molecule (e.g., nitric oxide), a metal or an oxide thereof, and an inorganic material.

As used herein, “biological fluid” or “sample” or “biological sample” can include any fluid or sample obtained from a subject (human, mouse, mammal, or animal model of a disease, e.g., lung cancer), including lung expiration, biopsy, or blood sample, a fraction or sample prepared from any sample obtained from a subject. As used herein, a subject can be an animal, mammal, or a human. In some cases, the sample may be treated or processed before being applied in an iCAP assay. For example, plasma or serum obtained from a subject may be treated or processed to remove albumin in order to provide a cleaner test substrate. In some cases, cellular extracts derived from a tissue sample can be used as a sample in iCAP. Thus, “biological fluid” can be understood to include fractions or samples of a tissue, cells, or fluids obtained from or derived from a subject. iCAP can be adapted for use with any biological fluid or sample. In addition to serum, plasma, cell lysate, or cerebrospinal fluid or fractions thereof, other fluids that may be tested include, but not limited to, semen, urine, saliva, and bile.

Biological fluids can be processed after collection. Processing of biological fluids can include centrifugation (e.g., differential centrifugation, rate-zonal centrifugation, isopycnic centrifugation, or other density gradient centrifugation). Processing of a biological fluid can include concentrating, removing, isolating, and/or diluting individual components of the biological fluid or groups of components of the biological fluid.

An indicator dye (such as calcein AM or ethidium homodimer-1) can be added to the biological fluid, e.g., to be used during response pattern characterization. In other embodiments, biological fluids from other lots and/or patients can be added to a given biological fluid. In yet another embodiment, substances (e.g., cryoprotectants) can be added to a biological fluid to aid in storage. In some cases, a biological fluid can be chilled, heated, or frozen during handling or storage. In some cases, a biological fluid can be analyzed for its properties (e.g., viscosity, specific gravity, etc.) or components (e.g., proteins, nucleic acids, pH, etc.) during handling, prior to use in iCAP systems, or during use in iCAP systems.

The subjects from which the biological fluids are obtained may be mammals, including primates, such as humans and animal models of lung cancer or lung disease, such as primates, rabbits, rats, and mice, as well as livestock such as sheep, goats, horses, cattle, and pigs and companion animals such as dogs and cats. The methods of the invention may be particularly useful in combination with model systems for disease, and in testing the effects of various therapeutic protocols thereon.

### Methods of Detecting a Lung Cancer

The present disclosure contemplates methods of detecting lung cancer or determining risk for lung cancer in a subject, the method comprising contacting a plurality of lung indicator cells with a biological fluid of said subject and comparing expression pattern in the indicator cells to that obtained when the indicator cells are contacted with a biological fluid from a normal subject, wherein an alteration in the expression pattern of the indicator cells contacted with the fluid from the subject as compared to indicator cells contacted with fluid from a normal subject determines a probability that said subject has lung cancer. Such methods can comprise all or a portion of an indicator cell assay platform (iCAP) assay (e.g., which may be referred to as an “indicator cell assay”, or a “cellular response assay” in some cases).

In some cases, a method of detecting or diagnosing lung cancer can comprise one or more of the following steps: a) contacting a first culture of responder cells with a biological fluid, or fraction thereof, from at least one diseased subject known to have lung cancer; b) determining a first response pattern of the first culture of responder cells to the biological fluid or fraction thereof by measuring levels of gene products, metabolites, biomarkers, or secretions of the first culture of responder cells, the first response pattern comprising a multiplicity of elements; c) contacting a second culture of responder cells with a biological fluid, or fraction thereof, from one or more subjects not having any lung cancer or by culturing the second culture of responder cells in the absence of extraneous biological fluid; d) determining a second response pattern of the second culture of responder cells to the biological fluid or fraction thereof by measuring levels of gene products, metabolites, biomarkers, or secretions of the second culture of responder cells; e) subsequent to steps a) through d) above, identifying elements of the first response pattern that differ from corresponding elements in the second response pattern as representing a third, differential, response pattern characteristic of lung cancer as compared to a lack of lung cancer; f) contacting a biological fluid, or fraction thereof, from the test subject (e.g., one in need of a diagnosis or confirmation of lung cancer status or type of lung cancer one has) with a third culture of responder cells; g) determining a test response pattern of the third culture of responder cells to the biological fluid or fraction thereof by measuring levels of gene products, metabolites, biomarkers, or secretions of the third culture of responder cells; h) comparing the third, differential, response pattern as determined in step e) with the test response pattern as determined in g) by determining, for the respective elements of the third, differential, response pattern as determined in step e), the levels of the corresponding elements in the test response pattern as determined in g); and i) detecting lung cancer in the test subject if the levels of the corresponding elements in the test response pattern as determined in step g) are statistically similar to the third, differential response pattern as determined in step e), wherein the responder cells are contained in at least one culture of cells of a type associated with the disease of interest or lung cancer; and wherein the elements of the (i) first response pattern, (ii) second response pattern, (iii) third, differential, response pattern, and (iv) test response pattern are elements of a transcriptome, proteome, metabolome, or secretion profile of the responder cells (e.g., protein, mRNA, RNA, DNA modification, DNA methylation, cytokine, or cellular byproduct). In some cases, an indicator cell can be referred to as a responder cell.

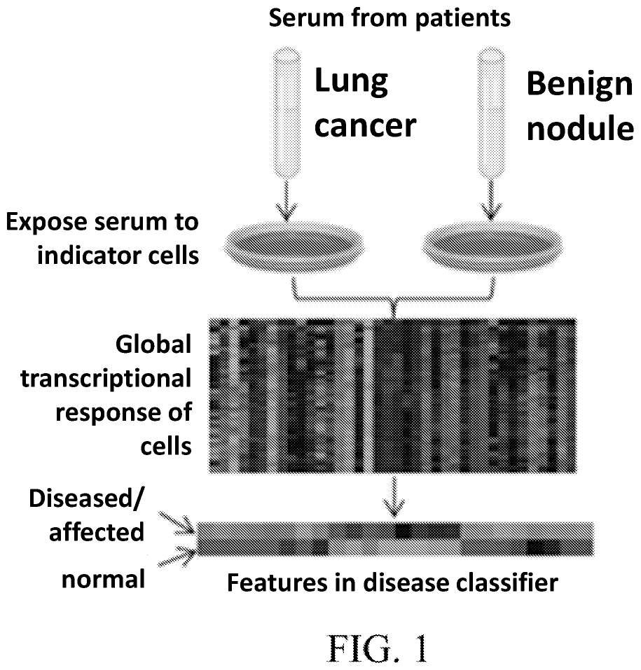

FIG. 1 shows a diagram of a representative example of the iCAP system, involving exposing standardized, cultured cells to serum from patients with cancerous cells, e.g., lung cancer, or benign nodules, identifying a global differential cellular response (e.g., differential response pattern) to the serum, and using disease classification tools (e.g., a classifier) to identify a subset of features for classifying and diagnosing disease state of patients. Shades of gray in the cellular response output data reflect levels of gene expression.

In some cases, the combination of performing a CT scan (e.g., via one or more feature comprising data from the CT scan) and using a lung cancer iCAP can be used to diagnose or screen patients who have or may have one or more indeterminate pulmonary nodule (IPN) (e.g., a non-calcified nodule). In some embodiments, an iCAP system or method can comprise (e.g., optionally, in combination with performing a CT scan) determining the presence of and/or the risk of lung cancer from one or more IPNs having a diameter of at least 3.0 mm, at least 4.0 mm, at least 5.0 mm, at least 6.0 mm, at least 7.0 mm, at least 8.0 mm, at least 9.0 mm, at least 10.0 mm, at least 11.0 mm, at least 12.0 mm, at least 13.0 mm, at least 14.0 mm, at least 15.0 mm, at least 16.0 mm, at least 17.0 mm, at least 18.0 mm, at least 19.0 mm, at least 20.0 mm, at least 21.0 mm, at least 22.0 mm, at least 23.0 mm, at least 24.0 mm, at least 25.0 mm, from 7 mm to 20 mm, from 6 mm to 10 mm, from 6 mm to 12 mm, from 6 mm to 15 mm, from 6 mm to 20 mm, from 6 mm to 30 mm, from 3 mm to 25 mm, from 4 mm to 25 mm, from 3 mm to 20 mm, from 4 mm to 20 mm, from 5 mm to 20 mm, from 5 mm to 25 mm, or from 5 mm to 30 mm. In some embodiments, an iCAP system or method can comprise (e.g., optionally, in combination with performing a CT scan) determining the presence of, absence of, or risk of malignancy for one or more IPNs having a diameter of at least 3.0 mm, at least 4.0 mm, at least 5.0 mm, at least 6.0 mm, at least 7.0 mm, at least 8.0 mm, at least 9.0 mm, at least 10.0 mm, at least 11.0 mm, at least 12.0 mm, at least 13.0 mm, at least 14.0 mm, at least 15.0 mm, at least 16.0 mm, at least 17.0 mm, at least 18.0 mm, at least 19.0 mm, at least 20.0 mm, at least 21.0 mm, at least 22.0 mm, at least 23.0 mm, at least 24.0 mm, at least 25.0 mm, from 7 mm to 20 mm, from 6 mm to 10 mm, from 6 mm to 12 mm, from 6 mm to 15 mm, from 6 mm to 20 mm, from 6 mm to 30 mm, from 3 mm to 25 mm, from 4 mm to 25 mm, from 3 mm to 20 mm, from 4 mm to 20 mm, from 5 mm to 20 mm, from 5 mm to 25 mm, or from 5 mm to 30 mm

In some cases, a combination of performing a CT scan (e.g., via one or more features comprising data from the CT scan) and using a lung cancer iCAP can be used to determine the presence of one or more IPNs. In some cases, an iCAP system or method described herein can be used to predict a presence of, absence of, or risk of malignancy of lung nodules identified by CT scan having a pretest risk of malignancy of at least 5%, at least 6%, at least 7%, at least 8%, at least 9%, at least 10%, at least 20%, at least 30%, at least 40%, at least 50%, at least 60%, less than 60%, less than 50%, less than 40%, less than 30%, less than 20%, less than 10%, less than 9%, less than 8%, less than 7%, less than 6%, less than 5%, from 5% to 60%, from 6% to 60%. Determining a risk of malignancy of one or more IPNs can comprise determining the number of IPNs present. Determining the risk of malignancy can comprise determining the density, size, shape, and/or texture of one or more IPN. Determining a risk of malignancy of one or more IPNs can comprise determining changes in the density, size, shape, spatial location, and/or texture of one or more IPN over time. In some cases, determining the risk of malignancy can comprise determining the size and growth rate of one or more IPN. In some cases, the determination of an IPN's density can result in the IPN being determined to be a soft tissue nodule, a ground glass nodule, or a semi-solid nodule. In some cases, determining the risk of malignancy can comprise use of an iCAP system or method comprising one or more response pattern features comprising data pertaining to age, presence of symptoms, cancer history, current or past smoking history, impaired lung function, history of exposure to environmental or occupational toxins or ionizing radiation (e.g., asbestos, radon, or uranium), genetic predisposition and/or low consumption of fruits and vegetables.

In some cases, a patient having a risk for lung cancer is first screened using an imaging diagnostic, such as a CT scan, followed by a lung cancer iCAP. In some cases, iCAP can be performed before an imaging diagnostic or without any imaging diagnostic. In some cases, lung cancer iCAP is a companion diagnostic for a lung cancer therapy or treatment. In some cases, a patient, characterized as having a nodule or a risk for lung cancer, such as a nodule previously identified using an imaging tool, e.g., CT scan, is administered a lung cancer iCAP or subjected to testing using an iCAP, to determine whether the nodule identified is benign, requires further testing, e.g., biopsy, or is at high risk for lung cancer. In some cases, iCAP can provide information on the presence of lung cancer, stage of lung cancer, and/or type of lung cancer to inform treatment decisions. In some cases, iCAP is a companion diagnostic that allows one to profile or determine the specific type or sub-type of lung cancer in a patient or whether a patient falls within a subset of population for which a therapy is indicated or known to be efficacious. In some cases, a patient undergoes or is administered iCAP testing before a therapy is administered or prescribed. In some cases, iCAP is used to track the progression of lung cancer or to monitor the health status of a patient, e.g., improvement over time following administration of a therapy.

In some aspects, the methods and diagnostics described herein can be used to characterize a suspicious nodule identified by CT scan to determine a probability of disease in a patient using a continuous variable.

In some embodiments, lung cancer is pre-diagnostic, pre-symptomatic, or pre-invasive lung cancer. Lung cancer also refers to any one of non-small cell lung cancer, small cell cancer, adenocarcinoma, squamous cell carcinoma, mesothelioma, and large cell carcinoma. In some embodiments, a subject is screened for a presence of nodules, such as indeterminate pulmonary nodule (IPN), using an imaging tool, such as a CT scan or x-ray. In some embodiments, subjects with IPN of 3-25 mm, 4.8-25 mm, or 6-25 mm are further tested using the cellular response methods described herein to further determine whether the IPN is benign, or malignant or non-benign, or at risk for developing cancer.

In some embodiments, a method described herein involving an iCAP can be performed at least two different times with the biological fluid, or fraction thereof, taken from the test subject at least two different times in order to determine the progression of lung cancer or a change in the subject's lung cancer status, including responsiveness to or a change resulting from a drug or a therapy. In some cases, a method described herein involving an iCAP can be performed at least two times with the biological fluid, or fraction thereof, from the test subject treated with a protocol, wherein the method can be performed before and after treatment with the protocol to determine effectiveness of the protocol.

In some embodiments, a set of differential response pattern features of a differential response pattern can be determined or generated by comparing response patterns of indicator cell cultures contacted with a positive control (incubated/contacted with a biological sample known to have lung cancer) and a negative control (incubated/contacted with a biological sample known to be negative for lung cancer). Response patterns from positive and negative controls (e.g., comprising response pattern feature values measured during experiments involving contacting an indicator cell population with either a positive control or negative control sample) are compared to identify elements or factors (e.g., features) that allow for efficient discrimination between a negative and a positive sample (e.g., elements of a transcriptome, proteome, metabolome, or secretion profile of the responder cells), or a differential response pattern comprising such elements or factors. For example, including measured or detected indicator cell parameters (e.g., features) that are strongly divergent between experiments in which the indicator cells are contacted with a positive control sample versus experiments in which the indicator cells are contacted with a negative control sample in the set of differential response pattern features can improve the accuracy and/or precision of an iCAP system or method. These parameters (e.g., features, which can also be called elements or factors) can be evaluated individually or in combination. In some cases, 5 or more, 10 or more, 15 or more, 20 or more, 30 or more, 40 or more, 50 or more, 60 or more, 70 or more, 80 or more, 90 or more, 100 or more elements or factors are identified and/or used to evaluated response patterns from indicator cells. Advantages of using such differential response patterns can include that one does not need to identify or have knowledge of the elements or factors beforehand in order to use the iCAP or methods of use thereof. This advantage can allow one to use the iCAP or methods of use thereof without any prior knowledge of biomarkers associated with a disease (e.g., lung cancer). Once a differential response pattern (e.g., comprising a plurality of differential response pattern features) has been generated, one can generate a test response pattern from a sample obtained from a test subject using indicator cells (e.g., comprising the same set of features as the differential response patterns, for example, populated with test response pattern feature values obtained from contacting a population of indicator cells with the test sample). The test response pattern feature values can be compared to the differential response pattern values (from the positive and negative controls), e.g., to determine how similar the values of the test response pattern features are to the positive control response pattern feature values (e.g., the response pattern feature values determined using a positive control sample) or the negative response pattern feature values (e.g., the response pattern feature values determined using a negative control sample). A statistically significant similarity between the test response pattern values and a negative response pattern values can suggest the test subject is negative for lung cancer, while a statistically significant similarity between the test response pattern and a positive response pattern suggests presence of lung cancer. In some cases, comparing response patterns for a statistically significant difference can refer to statistically significant difference in the measured levels of the elements/factors in the response pattern, e.g., level of mRNA, expression level of a protein or a biomarker, level of DNA methylation, level of a post-translational modification on a protein, or level of a cellular metabolite. In some cases, comparing response patterns (e.g., response pattern feature values) for a statistically significant similarity can refer to statistically significant overlap in the measured levels of the elements/factors in the response pattern, e.g., level of mRNA, expression level of a protein or a biomarker, level of DNA methylation, level of a post-translational modification on a protein, or level of a cellular metabolite.

In some embodiments, each response pattern can be generated using a culture of indicator cells. In some instances, indicator cells are of the same cell type relevant to the disease of interest, such as cultures of lung cells for detecting lung cancer. In some cases, more than one cell type can be used in an indicator cell assay. For example, multiple differential response patterns can be generated using separate cultures of bronchial epithelial cells and stem cells as indictor cells. In some cases, use of multiple indicator cell types can increase the specificity and sensitivity of the indicator cell assay to allow more accurate diagnosis and/or earlier diagnosis of lung cancer. In some embodiments, data from one or more individual elements/factors are combined and evaluated for significance as a group (e.g., hierarchical clustering). In some embodiments, measurements from multiple elements are compressed into a single value, or a smaller number of values to reduce dimensionality (such as principle component analysis), and significance can be measured for the compressed values. In some embodiments, statistical significance can be measured with a p-value (e.g., p<0.01, p<0.005, p<0.001, p<0.0005, or p<0.0001), a false discovery rate (FDR), or a confidence interval.

As described herein, lung cancer or disease can include, but may not be limited to, lung carcinoma, small-cell lung carcinoma (SCLC), non-small-cell lung carcinoma (NSLC), adenocarcinoma, adenocarcinoma in situ (AIS), or bronchioloalveolar carcinoma (BAC), squamous cell carcinoma, large cell carcinoma, mesothelioma, and large cell neuroendocrine tumor. Lung cancer can also include other cancers or tumors that have metastasized to the lung. Lung disease can include a disease or condition where lung cell or tissue is impaired including sarcoidosis, idiopathic pulmonary fibrosis. In some cases, lung cancer can be pre-invasive or pre-symptomatic.

Clinical tools for early diagnosis of lung cancer amongst high-risk patients include chest computed tomography (CT) scan, which reduces the relative risk for lung cancer deaths by 20% compared to chest X-ray. CT scans can be expensive for some patients. In some cases, CT scans can be cost-effective for screening patients that are of the highest risk for lung cancer. In some cases, CT scans can identify 63% of patients with early-stage cancer. In some cases, CT scans can have a high false positive rate (FPR) of 96%. In some instances, CT scans, especially when used alone, can result in over diagnosis of indolent cancers. Over diagnosis of cancer or false positives can lead some patients with benign nodules to undergo unnecessary and invasive follow-up testing, such as a biopsy to rule out cancer. Thus, there is a need in the field for a low-cost, non-invasive assay to improve lung cancer detection by indicating which patients should undergo CT scan and/or identifying which patients with positive CT results should undergo further diagnostic tests. In some cases, such non-invasive assay is used before or after, or used in conjunction with a CT scan, to improve the accuracy of diagnosis and/or improve early diagnosis or detection of lung cancer.

In some cases, compositions and methods disclosed herein contemplate a non-invasive blood-based test that helps classify indeterminate pulmonary nodules (IPNs) detected by CT scans. Such IPNs can be any nodule detectable with an imaging tool, e.g., CT scan, and where the pathology of the nodule has not yet been determined. Such IPNs can be benign, cancerous, or have a risk of becoming cancerous. In some cases, using such non-invasive blood-based cellular response assays in combination with CT scans reduces cancer deaths as compared to using CT scan alone and/or reduces the costs and morbidity associated with unnecessary follow up procedures as compared to using CT scan alone. Cellular response assays described herein provide an approach to further classify or determine the risk of such IPNs without invasive testing procedures, e.g., biopsy. In some cases, such cellular response assays can be used to confirm a nodule negative for cancer or malignancy or to confirm a positive diagnosis for cancer or a malignant nodule.

### Systems

An iCAP system can comprise a computer with a non-transitory memory on which instructions are stored, which when executed cause a processor of the computer to perform the methods or individual method steps disclosed herein. In some cases, an iCAP system can be used to determine a risk for lung cancer in a subject (e.g., based on a first response pattern and a second response pattern).

An iCAP system can comprise a population of cells (e.g., a population of indicator cells). In some cases, an iCAP system can comprise a first population of indicator cells. An iCAP system can comprise a plurality of populations of indicator cells. For example, an iCAP system can comprise a second population of indicator cells, a third population of indicator cells, and/or one or more additional indicator cell population.

In some cases, an iCAP system comprises a sample from a first subject, for example, to be used to contact a first indicator cell population (e.g., in determining a first response pattern). In some cases, an iCAP system comprises a sample from a second subject, for example, to be used to contact a second indicator cell population (e.g., in determining a second response pattern).

An iCAP system can comprise a classifier, as described herein. In many cases, an iCAP system can use a classifier to determine a differential response pattern (e.g., from a first response pattern, a second response pattern, a third response pattern, and/or one or more additional response patterns). For example, an iCAP classifier of an iCAP system can be used to create a differential response pattern based on a first response pattern (e.g., determined by detecting a first signal from a first population of indicator cells) and a second response pattern (e.g., determined by detecting a second signal from a second population of indicator cells). In many cases, an iCAP system can use a classifier to determine a set of key response pattern features (e.g., using a first response pattern, a second response pattern, a third response pattern, and/or one or more additional response patterns). In some cases, a classifier of an iCAP system can be used to determine a set of key response pattern feature values (e.g., based on the set of key response pattern features and a set of response pattern feature values, for example, of a first, second, third, or additional response pattern). As described herein, a classifier of an iCAP system can be a supervised, semi-supervised, or unsupervised classifier. In some cases, a classifier of an iCAP system is an ensemble classifier, as described herein.

An iCAP system can comprise an imaging module. An imaging module of an iCAP system can comprise a detector for measuring values (e.g., for use as response pattern feature values) from an iCAP assay (e.g., an experimental assay in which a population of indicator cells are assayed). In some cases, an imaging module comprises a lens, a stage (e.g., a motorized stage), and or a heating block (e.g., a thermocycler). In some cases, an iCAP system can be used to operate the imaging module. In some cases, an iCAP system can be used to operate the imaging module to detect one or more signals from an indicator cell population. For example, one or more response pattern feature value (e.g., of a first response pattern, a second response pattern, a third response pattern, or one or more additional response patterns) can be measured or determined by operating the imaging module. In some cases, comprises operating the imaging module to detect the second signal after the second indicator cell population is contacted with the sample from the second subject. In some cases, the imaging module can be used to detect one or more signals from an indicator cell population after an indicator cell population (e.g., a first, second, third, or additional indicator cell population) is contacted with the sample from a subject (e.g., a respective first, second, third, or additional subject). In some cases, operating the imaging module can comprise performing an RNA-seq assay, a reporter gene assay, a polymerase chain reaction (PCR) assay, an enzyme-linked immunosorbent assay (ELISA), next-generation sequencing, direct nucleic acid detection with molecular barcodes, microarray analysis, analysis of cell morphology, fluorescence microscopy, cell viability, or any combination thereof.

### Assays or Diagnostics

In some cases, iCAP systems, methods, and diagnostics described herein can provide for an assay with a high negative predictive value (NPV) and/or low false negative rate (FNR) to minimize the number of patients with malignant tumors that have negative test results. In some cases, the methods and diagnostics described herein can have intermediate specificity and false positive rate (FPR) and provide actionable results to patients by correctly identifying benign nodules or distinguishing benign nodules from malignant or cancerous nodules. In some cases, the methods and diagnostics described herein can have positive impacts on economics, e.g., lower the cost of diagnosis and/or allow early detection and treatment of lung cancer. In some cases, the methods and diagnostics described herein can have clinical utility and superior performance compared to other assays, such as CT scan alone.

In some embodiments, methods and diagnostics described herein can have ≤5% FNR (95% sensitivity), ≤40% FPR (60% specificity), and/or >90% NPV, or any combination thereof. In some embodiments, methods and diagnostics described herein can have a false negative rate of ≤5%, ≤4%, ≤3%, ≤2%, ≤1%. In some embodiments, the methods and diagnostics described herein can have a sensitivity of at least 90%, at least 91%, at least 92%, at least 95%, at least 96%, at least 97%, at least 98%, or at least 99%. In some embodiments, methods and diagnostics described herein can have a false positive rate of less than or equal to, or no more than: 40%, 35%, 30%, 25%, 20%, 15%, 10%, 5%, 4%, 3%, 2%, or 1%. In some embodiments, the methods and diagnostics described herein can have a specificity of at least 20%, at least 30%, at least 35%, at least 36%, at least 37%, at least 38%, at least 39%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least or at least 99%. In some embodiments, the negative predictive value (NPV) can be at least 80%, 85%, 90%, 91%, 92%, 93%, 94%, 95%, 96%, 97%, 98%, or 99%. In some embodiments, the positive predictive value (PPV) can be at least 40%, at least 45%, at least 50%, at least 55%, at least 60%, at least 65%, at least 70%, at least 75%, at least 80%, at least 85%, at least 90%, or at least 95%. In some embodiments, the overall detection rate can be at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%. In some embodiments, the overall detection rate of lung cancer can be 60-70%, 60-75%, 60-80%, 70-80%, 70-85%, 70-90%, 75-85%, 75-90%, 75-95%, 80-90%, or 80-95%. In some embodiments, iCAP systems, methods, and diagnostics described herein can have an accuracy rate of at least 60%, at least 65%, at least 70%, at least 75%, at least 80%, at least 85%, at least 90%, or at least 95% in detecting lung cancer or distinguishing IPNs as measured by cross-validation or using a hold-out set or independent samples. In some embodiments, iCAP systems, methods, and diagnostics described herein can have a sensitivity of at least 95% and a specificity of least 45%. In some embodiments, iCAP systems, methods, and diagnostics described herein can have a negative predictive value of at least 90%.

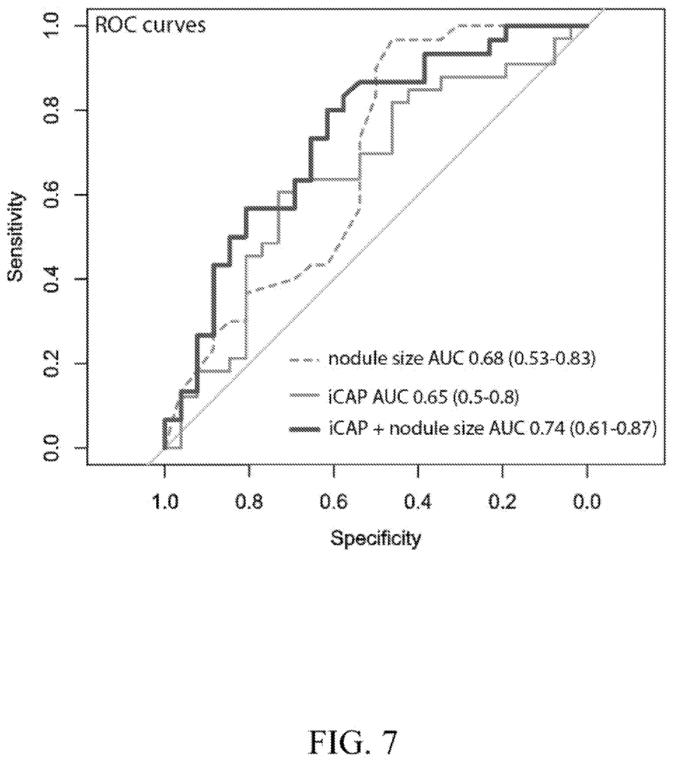

In some embodiments, an iCAP system can be a robust blood-based assay to distinguish patients with benign nodules from those with non-small cell lung cancer (NSCL), which represents about 85% of all lung cancer diagnoses. iCAP systems and methods can yield similar performances with a hold-out test and by cross-validation. Validation with a hold-out set can yield a ROC curve AUC of 0.74, and a cutoff approaching clinical utility with 92% sensitivity and 38% specificity. See FIG. 7.

In some embodiments, iCAP systems and methods can achieve low risk of missing malignant tumors (8% FNR), and actionable results for 38% of patients with benign nodules. In some embodiments, iCAP systems and methods can comprise FNR of less than 10%, 8% 5%, 4%, 3%, 2%, or 1%. In some cases, iCAP systems and methods can comprise sensitivity of at least 90%, at least 95%, at least 96%, at least 97%, at least 98%, at least 99%, or more than 95%, more than 96%, more than 97%, more than 98%, more than 99%, or more than 99.5%. In some embodiments, iCAP systems and methods can comprise FPR of less than 65%, less than 60%, less than 50%, less than 40%, less than 35%, less than 30%, less than 25%, less than 20%, less than 15%, less than 10%, or less than 5%. In some cases, iCAP systems and methods can comprise specificity of at least 60%, at least 65%, at least 70%, at least 75%, at least 80%, at least 85%, at least 90%, at least 95%, or more than 60%, more than 65%, more than 70%, more than 75%, more than 80%, more than 85%, more than 90%, more than 95%, more than 96%, more than 97%, more than 98%, or more than 99%.

Parameters of iCAP systems and methods can be enhanced and validated with cohorts from multiple independent sites, e.g., to further improve and validate the accuracy of the assay.

In some embodiments, iCAP systems and methods can achieve at least 94% accuracy (or at least 90%, 91%, 92%, 93%, 94%, 95%, 96%, 98%, 99%, or 99.5% accuracy). In some embodiments, the iCAP systems and methods can achieve at least 90%, 91%, 92%, 93%, 94%, 95%, 96%, 98%, 99%, or 100% sensitivity. In other aspects, iCAP systems and methods disclosed herein can achieve at least 40%, 45%, 50%, 55%, 60%, 65%, 70%, 75%, 80%, 85%, 90%, 92%, 94%, 96%, 98%, or 100% specificity in detecting affected versus unaffected samples in an independent test set. In some embodiments, an iCAP using human plasma or serum samples can be capable of at least 90% sensitivity and at least 95% specificity in validation with a hold-out set; or at least 90%, 91%, 92%, 93%, 94%, 95%, 96%, 98%, 99%, or 100% sensitivity and at least 40%, 45%, 50%, 55%, 60%, 65%, 70%, 75%, 80%, 85%, 90%, 92%, 94%, 96%, 98%, or 99% specificity in validation with a hold-out set.

In various aspects, the present disclosure provides an Indicator Cell Assay Platform (iCAP) that can use cultured cells as biosensors. In some cases, using cells as biosensors, as described herein, can capitalize on the ability of cells to respond differently to signals present in the serum (or other biofluid) from normal or diseased subjects with exquisite sensitivity. Advantages of indicator cell assays such as these make them better and more sensitive than traditional assays, e.g., which rely on direct detection of molecules in blood. In some aspects, the iCAP can involve exposing cultured cells to serum from normal or diseased subjects and/or measuring either a global differential response pattern or the response pattern of only a subset of elements or processes. A differential response pattern can be any detectable cellular difference that allows one to distinguish between the affected and unaffected biofluids. Affected biofluid can be from a subject with a physiological state or condition of interest, such as lung cancer. In some cases, affected biofluid (e.g., biofluid from an affected subject, for example, a subject with a high risk of having lung cancer) can contain substances indicative of lung cancer development and/or substances that can produce a response in one or more indicator cell that is indicative of lung cancer or a risk for lung cancer. A difference or change in response pattern feature values can be a difference or change in RNA, DNA, protein, gene expression, transcription level, and/or one or more lung cancer biomarkers in the indicator cells.

In some aspects, a reliable disease classifier (e.g., trained to compare samples based on response patterns comprising features, preferably a small number of features selected for their contribution to overall predictive power of the system) can be developed for use in an iCAP system, or a method of use thereof, and can be developed (e.g., trained) using iCAP. In some aspects, deploying the iCAP can involve measuring expression of one or more genes that are features of the disease classifier (e.g., using cost-effective tools such as RNA-Seq or PCR).

In some aspects, indicator cells can be chosen based on the disease application. In some aspects, indicator cells can be selected based on known relationships to a disease pathology.

In other aspects, the iCAP can overcome barriers to blood-based diagnostics like broad dynamic range of blood components, low abundance of specific markers, and high levels of noise. The sensitivity and/or the specificity of the iCAP approach can rely on the selection of the indicator cell type and the use of clonal cell populations derived from stem cells. Benefits of measuring the response of cultured cells compared to direct detection in a human sample can include normalization, buffering, amplification, transformation, and integration that a cell line provides.

In some aspects, iCAP can be used to diagnose patients who present with an indeterminate pulmonary nodule (IPN) detected on imaging to rule out benign nodules. The iCAP-lung cancer test can provide better performance with higher specificity and sensitivity when evaluating patients who first present with IPNs as compared to existing technology. In some aspects, an iCAP test for lung cancer can provide: i) identification of patients who present with IPNs who have minimal risk of having lung cancer, ii) non-invasive blood-based biomarker interrogation and identification, and iii) lower cost.

In some aspects, iCAP can leverage biological complexity and survey all serum molecules and their combinations that are detected by indicator cells. This can shift the paradigm in blood diagnostics from monitoring a few molecules to capturing complex disease signals with multicomponent readouts that can indicate disease with potentially better performance and earlier detection than other methods. For example, iCAP can detect lung cancer at early stages when diseased cell counts are too low for detection by conventional methods.

In some respects, diagnostic systems, methods, and compositions described herein can comprise cells that can translate a complex signal or pattern associated with a diseased or unhealthy cellular state into a detectable readout or a measurable response, such as differential gene expression, even when the nature of this signal or pattern is not known or understood. In some cases, this can be achieved by using a plurality of cells (e.g., indicator cells) as detectors, biosensors, or indicators such that a complex pattern associated with the indicator cells provides the readout of the assay. Since indicator cells (or responder cells) can themselves be complex systems, they can provide a multiplicity of parameters that can be measured substantially simultaneously to provide a detectable response pattern.

In some cases, application of this concept can involve employing indicator cells to assess complex changes in biological fluid of a subject when the condition of the subject deviates from normal. The presence of cancer, diseased, or abnormal cells, for example, can result in a change in the contents or composition of bodily fluids, for example, blood or cerebrospinal fluid (CSF). In some cases, when these fluids are contacted with indicator cells, the cells themselves can exhibit a response (e.g., endogenous signals such as qualitative or quantitative transcriptomic, proteomic, metabolomics, or lipidomic elements) that can constitute a response pattern, which can be detected or measured to determine the health status of the subject or risk of having or developing the disease, such as lung cancer.

In some aspects, an indicator cell assay can be used to determine a differential response pattern characteristic of an abnormal condition (e.g., a disease such as lung cancer) or a disease stage in a subject, for example, wherein the method comprises contacting a first sample of a culture of indicator cells with a biological fluid or fraction thereof of a subject known to have said abnormal condition or disease stage and determining a first response pattern of said cells to said fluid, contacting a second sample of said culture of indicator cells with the bodily fluid or fraction thereof of a normal subject or cells that have not been contacted with bodily fluid and determining a second response pattern of said indicator cells to said fluid, comparing the first response pattern with the second response pattern; and/or identifying elements or parameters of the first response pattern that differ from corresponding elements or parameters of the second response pattern as representing a third, differential, response pattern characteristic of the abnormal condition.

In some cases, the indicator cell assay system can be used to perform a method to detect an abnormal condition or disease stage in a subject by determining whether the subject has a differential response pattern characteristic of the abnormal condition or disease stage. In some cases, this can be accomplished by contacting a biological fluid or fraction thereof of said subject with indicator cells and determining a response pattern of said cells. The response pattern can then be compared to those of control cells which have not been contacted with said biological fluid, or that are contacted with the corresponding biological fluid of a normal control subject, or to a standard normal response pattern compiled from other subjects, which may be accessed in a database, in some embodiments. In some cases, this “normalized” differential response pattern can be compared to the differential response pattern determined as described above.

In some cases, it is also possible to compare the profile (e.g., response pattern feature values) obtained when a culture of indicator cells is exposed to a fluid sample obtained directly from one or more subjects with a known condition (e.g., physiological state) or with a particular stage of said condition with the profile (e.g., response pattern feature values) obtained by subjecting a second culture of cells to fluids or fractions from a subject with an unknown condition or disease stage. In some embodiments, similar profiles can indicate a correspondence of the condition or stage of the test subject with that obtained from the subject who is afflicted with the known condition or stage.

In some embodiments, iCAP systems and methods can measure complex responses of cultured cells in vitro to multiple external factors carried in blood and use them to assess disease state. iCAP cells can improve reproducibility of these assays. The use of terminally differentiated and genetically identical indicator cells that are reproducibly obtainable from a self-renewing, single source of stem or progenitor cells maintained under stringent conditions can reduce significant gene expression noise in the readout arising from genetic diversity of the individual subjects being assayed or diagnosed. The iCAP readout can also provide disease specificity. For example, an iCAP system or method can distinguish between diseases and disease subtypes in separate subjects or subject populations, in many embodiments. iCAP cells can have known responsiveness to extrinsic signals of disease and disease-specific response patterns (e.g., key response patterns or signatures).

In some cases, iCAP systems and methods can reveal mechanistic insights into a physiological state of interest (e.g., a disease or disease subtype) and/or its progression. iCAP can be an effective blood-based diagnostic tool for human diseases. Differentially expressed genes and gene sets (e.g., which can be response pattern features of an iCAP system or method) can be significantly enriched for those implicated in the disease processes, suggesting that the iCAP has additional utility for understanding disease mechanism and identifying candidate therapeutic targets. Success of the iCAP does not necessarily depend on understanding the biological responses. For example, the cellular roles of the genes in the readout may be irrelevant, in some cases, for example, if there is significant differential expression in response to disease versus normal serum or sample.

In some embodiments, iCAP systems and methods can capture cellular responses to complex signals of active disease generated in vivo, and thus can have relevance to understanding disease processes or progression (e.g., the spread of neurodegenerative disease and cancer pathologies to unaffected cells via secreted material). In some cases, iCAP systems and methods can be used to study disease-related genes and their pathways.

To optimize experimental parameters of the assay, preliminary data (e.g., as obtained under a standard or control condition in an assay, such as a cellular response assay) can be used to generate (e.g., train or build) disease classifiers and calculate the information added to the classifier from the replicates performed under each tested condition. Information-based methods can be used to evaluate the effect of the new experiments on the classifier using different criteria.

Various computer models for in silico predictions can be used to train classifiers that predict or enhance diagnosis of a disease or condition. For example, an active learning tool such as Maximum Curiosity can be used to improve classifier accuracy, while Minimum Marginal Hyperplane can be used in some cases to improve classifier confidence as encoded by the distance of new examples from the decision boundary. In some cases, modifying experimental conditions of the iCAP such as serum concentration and time of incubation of serum with indicator cells can also produce data that improve the classifier. Adding other data to the classifier (e.g., as values of a feature), such as nodule size, nodule location, smoking history, patient gender, genetic data, and environmental factors can improve accuracy and confidence of the iCAP system or method, in some cases. For example, if a particular sample was correctly classified as disease with 55% confidence before the new data was added, after the new data, the confidence may increase to 75%. In some cases, a regimen including an active learning trial, leave-one-out cross-validation, and repeated leave-two-out cross-validation can be used to design or enhance classifiers for diagnosis.

Aggregate increase in confidence or accuracy can be reflected in an increased area under the curve (AUC) measurement. In some embodiments, experimental parameters (e.g., measurable experimental metrics, which may be selected as features of a response pattern) can subsequently be selected by choosing those that most increase classifier AUC across multiple cross-validation runs when those experiments are added to the classifier. In some embodiments, the conditions that minimize false positives or false negatives can also be determined from this type of analysis. A condition-specific (e.g., disease-specific) response pattern (e.g., key response pattern or signature) generated (e.g., determined) for a given indicator cell type (and, optionally, for a given experimental condition) may be determined to be unique. This does not necessarily preclude testing the new conditions using this approach. For example, an iCAP system or method can be used to develop multiple response patterns or multiple key response patterns for analyzing the same physiological state (e.g., condition or disease, such as classifying samples into lung cancer and benign classes), for example, by using a different set of positive and/or negative control samples as an input to the system when developing the system. In some embodiments, no version of data from a left-out test sample is present in the training set.

In some cases, iCAP classifier performance can be optimized by testing experimental parameters in pairs. An advantage of testing parameters in pairs, rather than a greedy search where parameters are tuned sequentially can be that the paired parameter space may have several local minima, which would be partially revealed. By recording a larger sampling of the search space, more room for future refinements and cost-sensitive exploration of all promising parameter combinations can be provided.

For example, a matrix of 6 experimental parameters (e.g., features potentially useful for inclusion in a response pattern) for each of two cell types can be used and an optimal condition can be chosen based on improvement of the accuracy and/or precision of the key response pattern (e.g., disease signature) and/or the classifier performance. If both cell types have similar rankings, it can be beneficial to select endothelial cells derived from (e.g., differentiated from) stem cells (e.g., induced pluripotent stem cells, or iPSCs) as indicators over, for example, lung epithelial cells due to their level of suitability for clinical-stage development.

iCAP system and methods (e.g., for determining the presence, absence, or risk of lung cancer) can include optimization of several technical aspects to improve utility, including the collection and handling of patient biofluids, the use of RNA-seq instead of microarrays for global gene expression analysis, and the use of specific cell culture plates to control well-to-well variation.

Within-plate and/or between-plate variation can be monitored and/or corrected, e.g., by analyzing two iCAP plates, each with 6 reference serum replicates in edge, middle and corner plate positions, and by running and analyzing a single reference serum samples on every assay plate for the entire project. Reference data, e.g., for monitoring and/or correcting within-plate and/or between-plate variation, can be used for standard normalization and co-variate correction approaches. Variation can also be corrected by normalizing each gene expression value to a standard value derived from a subset of stably expressed or unresponsive genes in the same expression pattern.

Quality control filters to remove samples failing technical processing standards can improve classifier performance. Sample complexity can be monitored and assigned a threshold, which reflects the number of unique sequencing reads per sample, to flag problems with sequencing or library preparation. The complexity threshold can be 30%, and can be adjusted based on the distribution of the data. Grubb's outlier analysis of sample complexities can be applied to remove outliers from the dataset. To reduce technical variation, libraries can be prepared using a robot and failed samples can be re-prepared from stored RNA without the need to repeat the cell assay. Technical effects of library preparation and sequencing can be controlled for with RUV (e.g., “remove unwanted variation” normalization methods) or other RNA-seq normalization approach.

A QC threshold can be set for biovariance of each sample (e.g., correlation of top differentially expressed genes) and Grubb's outlier testing can be performed to flag samples that had technical failure. Samples that fail at a point after the cell assay can be reanalyzed from stored RNA without repeating the assay. Within- and between-batch intra-class coefficient of variation (CV) of gene expression can be monitored and quality controlled with co-variate correction. Uncorrected median of mean CVs for within- and between-batches can be 9.6% (e.g., +/−1.2%) and 19.9%, respectively, and co-variate batch correction can reduce between-batch CV to 10.6% (which can be within 1% and 1 standard deviation of the within-batch variation, suggesting successful correction).

In some cases, assay data generated from two different users, can be compared by duplicating cell-based assays for 10 samples (N=5 of each class) for classifier training. iCAP data can be generated using samples from 2 or more clinical sources to improve classifier robustness.

In some cases, measurement and control of potential technical variation from various sources including within and between batch variation, and variation from different assay users can be accomplished by adjusting culture and design aspects of the iCAP system.

In some cases, computational parameter optimization of the iCAP can be used to improve iCAP performance, including optimization of upstream data analysis such as the genome alignment method, normalization and covariate correction, and gene expression value transformation, and classification approaches, including the feature selection method, dimension reduction method, and machine learning or pattern learning approaches. Normalization can include within-sample normalization, for which expression of a gene is normalized to that of another gene(s) in the same profile, or within-batch normalization, for which expression of a gene in one sample is normalized to gene expression in another sample in the same experimental batch. The optimal computation parameters between lung cancer iCAP classifiers and those identified (e.g., generated and/or trained) for other diseases may be similar, allowing one disease model to inform or assist in selecting or identifying classifiers for another model. Batch specific effects can impact the lower limit of detection of gene expression. Using RNA spike-in controls, a threshold can be set to filter out genes with low expression levels leading to unreliable expression quantification.

Gene expression values from poly-adenylated transcripts as response pattern features (e.g., features used in a classifier) can be used for classification. Data can be from protein coding genes, as well as non-coding genes, which appear to represent 80% of transcription in mammalian genomes and may have important regulatory roles. RNA-seq approaches can also capture RNA splicing that can be informative to the classifier, feature types used for classification can be expanded to include 1) adding RNA splicing as a feature type, and 2) using different genome annotation libraries with improved annotation of non-coding transcripts.

Optimal prior probability of disease can be determined in a training set to maximize NPV (and reduce FNR), while still achieving a clinically useful specificity of 60%. An unbalanced iCAP-lung cancer training set with 75-80% malignant samples can enhance sensitivity over specificity, which is predicted to remain high when applied to the intended clinical population with 23% prevalence.

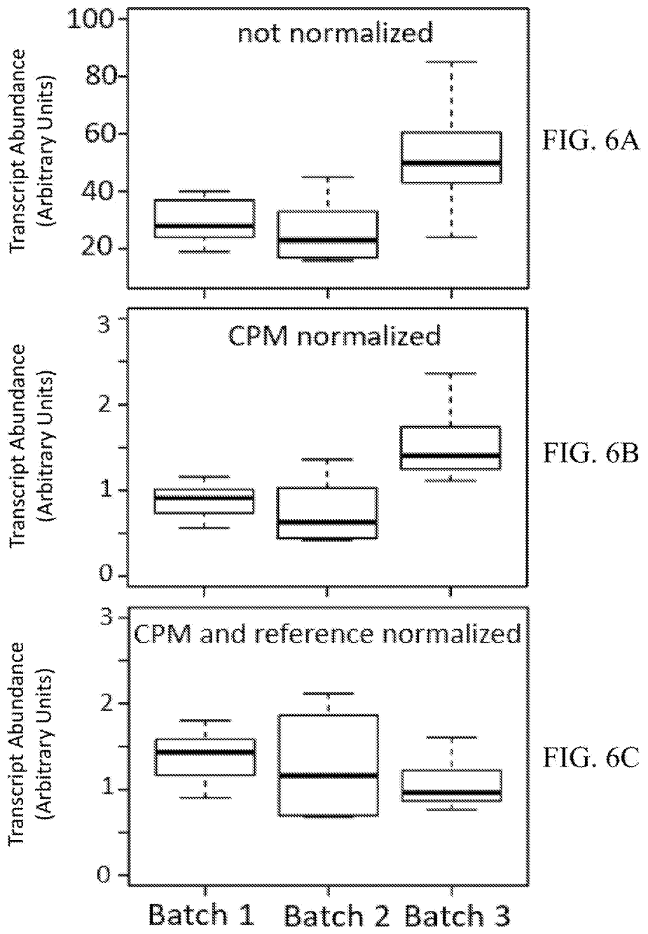

Co-variates in the iCAP, such as assay well position, or assay batch (which could lead to within- or between-batch variation, respectively) can be corrected for using open source co-variate correction software. However, including standard reference sample on each plate can provide powerful batch correction capabilities. The abundance of a randomly chosen transcript across three different iCAP batches can be compared, either without normalization or after either counts per million (CPM) mapped reads normalization, where the counts for each transcript are scaled by the number of fragments sequenced, or normalization by standardizing transcript abundance in the test sample to that in the reference sample on the same plate. Reference-normalized ratios can have less variation between batches than CPM alone. Average gene of interest expression data (e.g., WASH7P) obtained from three experiments and analyzed using three separate normalization methods (not normalized, CPM normalized, and CPM and reference normalized) are shown in FIG. 6A, FIG. 6B, and FIG. 6C, respectively.

After optimization of experimental parameters (e.g., a first response pattern), the iCAP-lung cancer classifier can be retrained and tested using optimal parameters (e.g., a key response pattern). The classifier can be generated using as few as 115 samples; to increase power, and to comprehensively test accuracy and robustness, sample size can be increased to 318 samples for training (e.g., data for 298 new samples can be generated using optimal parameters and merged with data for 20 samples that were used for optimization and generated using optimal parameters). Such a sample size (and the sample size used for testing) can be comparable to those used for other lung cancer diagnostic assays in development and may be advantageous given the large size of gene expression-based datasets. Data can be generated and analyzed in stages so that preliminary classifiers can be iteratively developed and tested against newer blind data. This approach can reduce likelihood of overfitting and establishes an accuracy trajectory that can be used to corroborate the number of replicates needed for a robust classifier. Importantly, this approach also can allow many computational approaches to improving or evaluating various parameters of the iCAP assay.

For each round of analysis, differentially expressed features (including genes and gene sets) can be used for feature selection or feature reduction to select the smallest subset of features that maximizes the number of informative features for classification. In some aspects, feature selection can be a multi-step process that involves initial user-directed feature selection, in some cases based on differentially expression and/or other attribute such as disease relevance, followed by automated model-based feature selection involving multiple iterations of classifier training. In some embodiments, there is no specific, pre-defined process of feature selection; it can be driven instead by an iCAP system user's understanding of pertinent biology pertinent to the physiological state or states of interest, the conditions and assumptions of the experimental system, and the statistical nature of the data produced during use or development of an iCAP system, composition, or method. Feature selection can be an important aspect of developing an iCAP in some embodiments. In some cases, inclusion of non-informative features in a model can result in dilution of key informative features and can increase the chance of overfitting, which can reduce the likelihood of the resulting system or method having robust performance on independent samples. In some cases, inclusion of highly correlated features (e.g., those with correlated profiles of expression across the samples) in model-based feature selection, can result in flat feature importance scores and may impede the ability to identify informative features.

Selected features can be used to train disease classifiers, exploring various approaches applied previously. To iteratively test robustness and improve accuracy, several rounds of classification with different parameters can be simulated as data are collected. Classifiers can be trained using 25 samples of each class, and accuracies can be tested against blind left-out data (25 of each class). Classifiers can also be trained with all data and tested by 10-fold cross validation. Classifiers can also be trained using iCAP data for samples in one experimental batch (6 samples of each class) and tested against data for the other samples (51 samples of each class). In such cases, the test samples (e.g., subject samples) can be independent and may not necessarily be used to train the classifier.

Advantageously, an iCAP can be based on biosensor data that is orthogonal to other patient data and other assays that directly detect molecules in serum. Therefore, clinical data (e.g. patient age, nodule size, and smoking history), or other response pattern feature data (e.g., cell parameter values) can be included in the classifier to improve iCAP performance (e.g., accuracy), in some embodiments. In addition, clinical or other data can be used to direct feature selection (e.g. features can be selected whose pattern of expression matches the pattern of tumor subclass or other clinical data). The data can also be explored by performing unsupervised classification of samples to identify unknown subclasses of the data that might correspond to different subclasses of the disease or patient status.

iCAP can comprise a robust disease classifier that can differentiate patients with benign nodules from those with non-small cell lung cancer (NSCL) with significant validated accuracy with ≤62% FPR and ≤8% FNR.

Given that iCAP classifiers can be based on global gene expression data, and the number of potential features can be much greater than the number of patient samples tested, consideration may be given to avoid overfitting. An iterative approach of retraining the classifier at various stages as new data are obtained can be used. In some cases, this allows for classifier testing with multiple configurations allowing one to recognize an increasing accuracy trajectory, an important measure of classifier robustness. Another measure to combat overfitting can be to ensure the number of features for classification is fewer than the number of samples used to train the classifier. If overfitting is a problem the number of potential features can be reduced, while minimizing information loss, e.g., by using gene sets as features instead of individual genes/transcripts. Gene sets can be related genes that have been grouped based on co-expression in other datasets, or their involvement in the same cellular process or another commonality.

A robust final classifier can be validated on intended use samples from two or more independent sites to achieve blind predictive accuracy corresponding to >90% NPV, e.g., to reduce the post-test probability of cancer to ≤10% amongst patients classed as benign, and/or to achieve an FPR≤40%, which would save 60% of those with benign nodules from further diagnostic testing.

In some embodiments, a new classifier can be generated with all available samples (e.g., at least 400, at least 300, 318, at most 300, at most 200, at most 100, at most 50, or at most 25 samples can be used to train a classifier, plus at least 200, at least 150, 165, at most 150, at most 100, at most 50, or at most 25 new independent samples) and tested by repeated 10-fold cross-validation. Increasing the size of training set can increase the accuracy of the classifier. The new classifier can be tested against a new, independent sample set. In addition, other non-iCAP features can be incorporated into the classifier. Including clinical assessments such as age, nodule size and smoking history into a classifier can improve accuracy.

To control for overfitting, iCAP systems and methods can comprise pathway analysis, which can involve assessing the significance of pre-defined gene-sets rather than individual genes, to reduce the multiple hypothesis testing problem due to the large number of genes in the genome.

A blood-based classifier with a 45% specificity can save almost half of the patients with benign nodules from further diagnostic evaluation including invasive biopsy. A very high 95% sensitivity would minimize the risk of misclassifying malignant tumors as benign. In some embodiments, the methods and diagnostics disclosed herein have at least 45% specificity and/or at least 90% or 95% sensitivity.

In some embodiments, an iCAP classifier can be developed to distinguish NSCL from benign nodules initially identified as IPNs by CT scan. Classifiers can be trained and/or tested against a left-out test set and by cross-validation with similar results demonstrating classifier robustness. The classifier can have clinically useful sensitivity and specificity values of 95% and 45%, respectively.

After selection of indicator cell types, the concentration of serum and exposure time in the iCAP can be evaluated for improving the sensitivity and/or specificity of the assay. Evaluation of improvements to sensitivity and/or specificity can be evaluated, for example, by: 1) testing for improvements to case versus control disease signature, including maximizing the number of differentially expressed genes, the magnitude of differential expression, and the enrichment of disease relevant genes, and minimizing within-class variance, and/or 2) testing for improvements to classifier performance. To do this, data for a given condition can be added to existing data and used to retrain the existing classifier. Improvements to classifier performance (accuracy and AUC) can suggest improved experimental conditions.

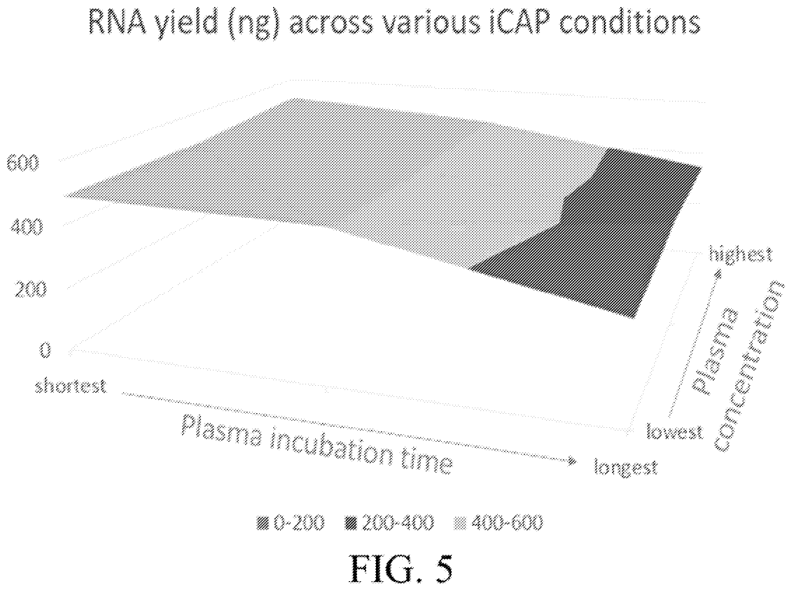

Plasma concentration and exposure time can be tested for multiple iCAP assays using factorial design. RNA yield can be inversely correlated with incubation time (Pearson correlation p-value <0.05). An example of results from a factorial experiment to evaluate plasma concentration and incubation time in an indicator cell assay, showing RNA yield (ng) plotted across various iCAP conditions is shown in FIG. 5. Parameters analyzed can include the number of significantly differentially expressed genes between disease and normal classes, within-class variance, culture health and enrichment of disease-related processes amongst differentially expressed genes. For the iCAP systems and methods, shorter incubations can lead to stronger disease signatures (e.g., higher magnitude of differential expression and number of differentially expressed genes). Within-class variability can be evaluated whether it falls in an acceptable range.

Diagnosis of a disease (e.g., lung cancer) can be useful in clinical and research settings, and iCAP systems and methods can be used to do so. Response patterns (e.g., first response patterns, second response patterns, differential response patterns, etc.) can be used to diagnose a disease (e.g., lung cancer) in part by, for example, comparing the response pattern generated by contacting an indicator cell with a biological fluid from a patient with the disease and the response pattern generated by contacting an indicator cell with a biological fluid from a patient that does not have the disease. Response patterns can also be compared longitudinally using the multiple aliquots of biological fluid from the same patient to identify and track disease progression or severity of disease.

In some aspects, a differential response pattern comprising iCAP systems and methods can be established by comparing responses obtained from fluids obtained from abnormal subjects with those obtained from fluids of normal subjects. The responses can be compared by identifying individual transcripts that are significantly differentially expressed between the two responses, or by generating and testing a more complex disease classification model using approaches such as support vector machines or random forest algorithms. Such algorithms can identify diagnostic signatures composed of sets of candidate transcripts and disease classification decision rules (which can be based on more complex aspects of the data such as the relative intensities of two different transcripts in the same sample).

The analysis can be expanded to obtain a longitudinal or cross sectional set of disease signatures, by obtaining complex multicomponent readouts from indicator cells (e.g., gene expression microarrays) after exposure to biological fluids (e.g., sera) from normal or diseased subjects taken at various stages of disease progression. In longitudinal studies, a single subject at various disease stages can be assessed, whereas in cross-sectional diagnoses, multiple subjects at various disease stages can be used as subjects. In some cases, a differential response signature can be the difference between the expression patterns of the same patient at two different stages of disease, or between expression patterns from different patients at different stages of disease.

In some embodiments, disease progression can comprise constructing a differential response pattern made up of log 2 expression ratios (disease serum exposure/normal serum exposure) obtained of indicator-cell genes in the cultured cells that are good indicators of disease progression. Expression values of various genes for disease subjects at each stage of progression can be evaluated relative to matched normal subjects (e.g., subjects can be matched with respect to genetics, age and/or environment). This can be a standard pattern to which expression level data obtained similarly with respect to fluids of a test subject can be compared. If a large number of genes make up the signature, it is possible, if so desired, to cluster genes in some way; for example, genes with similar response profiles can be clustered. Categorizing genes in this way can enable recognition of higher-level disease signatures such as response characteristic of a cellular process rather than expression level of an individual gene.

In some embodiments, iCAP assay can be performed with serum from the subject and disease state can be assessed by mapping to the longitudinal progression pattern. This can be done by obtaining readouts from indicator cells after exposure to query serum of a test subject and control serum using the same experimental conditions that were used to generate the longitudinal (progression) data. In some embodiments, the control can be non-disease serum from a genetically matched subject of the same age, but for other disease applications, it could be serum taken from the subject itself before disease onset.

In other aspects, the pattern obtained from various subjects representing the stages of disease progression can be compared directly with the expression pattern obtained from a test subject to compare similarities between their expression patterns.

In some embodiments, advantages of iCAP can include: 1) sensitivity—blood components of low abundance can elicit robust cellular responses; 2) specificity—the iCAP capitalizes on the naturally evolved ability of cells to detect specific signals in noisy environments, and the concept of a field effect in which presence of cancer is reflected by changes in distal tissue by secreted material; 3) captures complexity—cells naturally respond to a broad range of molecules (including proteins, nucleic acids, lipids and other metabolites, or combinations thereof). In some embodiments, the sensitivity and specificity can be any of the values or combination of values disclosed above.

In some embodiments, iCAP can allow for multicomponent gene expression readout from a genetically identical population of cells and early detection of a disease or condition, even when indicators are of low abundance at early stages, eliminating challenges faced by analysis of biomarkers directly in plasma or cells sampled from the subject due to variable abundances of particular cell types in blood, genetic variation between individuals, and prominent responsiveness of immune cells to generic inflammatory signals as opposed to true indicators of cancer or disease.

In some embodiments, the iCAP-lung cancer can be configured as a high throughput low cost assay and implemented as an assay for lung cancer diagnostics. The cell biology component of the test can be configured as manually manipulated 12-well plates or 96-well plates or 384-well plates with islands of automation. Automation can increase robustness and significantly reduce hands on time and reagent use. Manual RNA extraction can be replaced in iCAP systems and methods with automated RNA-Seq and medium-scale multiplexed RNA detection assays such as RNA-Seq and PCR platforms.

In some embodiments, iCAP can be used to reveal underlying mechanisms or pathways of a disease or condition. In some cases, iCAP is configured to capture cellular responses to complex signals of active disease generated in vivo, which can provide insights into disease processes.

In other aspects, iCAP can be used to further define or refine classification of complex diseases based on the underlying pathway or mechanism. Such refining of disease classification can inform treatment decisions. For example, subsets of patients with lung cancer can be better defined so that more targeted therapeutics can be prescribed. In some cases, iCAP can be used to define the appropriate patient population or subset within a patient population that is most likely to benefit from a clinical trial of a new therapy. In some cases, iCAP is used as a companion diagnostic to better target a therapeutic agent to the appropriate patient population or a subset of the population. In other cases, iCAP can be used to monitor patients over a course of a therapy, such as during a clinical trial, as a standard for monitoring efficacy of the therapy or as a surrogate outcome or endpoint. iCAP's sensitivity also allows one to better measure endpoints and monitor efficacy of a therapeutic agent in complex disease progressions. In some cases, iCAP systems and methods can be used to monitor drug efficacy in a subject, wherein efficacy of therapeutic agent can be measured as a change in the response pattern of indicator cells, e.g., a change from a late stage response pattern to an earlier stage response pattern.

In some embodiments, the methods and diagnostics described herein can be used in combination with any of the existing methods to increase accuracy of diagnosis, including but not limited to, CT scan, PET scan, bronchoscopy, thoracoscopy, pulmonary function tests, fine needle aspirate, surgery, biopsy, bronchoscopy and genetic testing, and multi-factorial protein blood test, or any combination thereof. In some cases, use of iCAP in conjunction with a CT scan can help to rule out or screen out low risk patients, who can avoid more invasive tests, such as biopsies. In some cases, iCAP can be used to confirm an IPN identified by CT as a malignant nodule and alert the subject to further, more invasive testing and treatment methods.

In some embodiments, the indicator cells or the cellular response assay can be provided in the form of a kit. In some embodiments, the kit can comprise a set for indicator cells or cell lines for detecting lung cancer. In some embodiments, a kit can comprise software for comparing expression patterns obtained from indicator cells with data of a control or a set of controls. In some embodiments, the software in the kit can contain an trained iCAP classifier and/or a list of iCAP response pattern features or iCAP key response pattern features for determining a subject's risk for developing lung cancer or for determining whether an IPN is benign. In some embodiments, classifiers and control data can be provided in a kit in the form of a computer program or as a database in the cloud, which can be accessed by a user for analysis and comparison to a response pattern of a test sample. In some embodiments, the kit can contain a device for collecting a biological sample, whereby a user can mail the sample to a laboratory for testing the sample using the cellular response assay described herein.

In some embodiments, a kit or a diagnostic system can comprise one or more cultures of indicator cells for contacting with a test sample, instructions for generating a test response pattern, and access to a software or database containing various positive response patterns, negative response patterns, and/or differential response patterns based on a plurality of patients with known/verified lung cancer risk and/or status. Statistical comparisons between the test response pattern and such database of previously characterized response patterns can allow one to determine presence of lung cancer-associated elements or factors in the test sample, lung cancer status of a test subject, risk of an IPN, prognosis and/or the effectiveness of a therapy. In some cases, such database of previously characterized response patterns can be used to validate and/or refine classifiers for detecting lung cancer, e.g., to increase signal-to-noise ratio in the response patterns or to increase sensitivity or specificity of iCAP.

### Treatments

Assessing disease stage can also be useful in evaluating treatment, and iCAP systems and methods can be used to do so. The expression pattern of indicator cells characteristic of a particular stage of a disease, whether or not normalized to that of normal subjects can be compared to the pattern obtained from a test subject before and after treatment, again, either directly or where both patterns have been normalized to normal subjects. Effectiveness of the treatment can be reflected in finding that the pattern in the test subject represents an earlier stage of progression than was exhibited before treatment. Thus, if before treatment the subject exhibits the pattern characteristic of stage 4 lung cancer, successful treatment can be indicated if the pattern after treatment is representative of disease stage prior to 4, such as disease stage 3 in some embodiments. In some embodiments, methods or diagnostics disclosed herein can distinction the various stages of cancer, e.g., stage 1, 2, 3, and 4 lung cancer.

iCAP systems and methods can be used for early detection of lung cancer or disease from human samples and can be tested with the complex genetic and environmental diversity of a human population. Diagnoses made using iCAP systems and methods as well as response patterns generated using iCAP systems and methods can be used to determine treatment options of patients in need thereof (e.g., patients with lung cancer).

In some aspects, the present disclosure contemplates using the iCAP system or cellular response assays disclosed herein as companion diagnostics for use with any imaging tool, diagnostic tool, and therapy for lung cancer. Also contemplated herein is a method of treating lung cancer, the method comprising screening a subject using a cellular response assay, comprising: contacting a plurality of lung indicator cells with a biological fluid of said subject and comparing expression pattern in the indicator cells to that obtained when the indicator cells are contacted with a biological fluid from a normal subject, wherein an alteration in the expression pattern of the indicator cells contacted with the fluid from the subject as compared to indicator cells contacted with fluid from a normal subject determines a probability that said subject has lung cancer; and treating the subject with a therapy known to be responsive to the lung cancer identified by the cellular response assay. In some embodiments, the subject can be screened before or after the cellular response assay using another method, such as an imaging tool, e.g., CT scan. In some embodiments, the cellular response assay is designed such that it screens for lung cancer biomarkers specific to a patient population for which a therapy is indicated, approved, or known to be efficacious. Using the cellular response assay as a companion diagnostic with a therapy can decrease unwanted side effects by decreasing off-target effects and/or allow for more targeted treatment so that the treatment is given to patients who are most responsive to the therapy.

When such methods and drugs are used as companion diagnostics in clinical trials, by selecting the subset of patient population with the appropriate biomarkers that are most responsive to a therapy, one can increase the statistical power of the clinical trial by selecting the most relevant or responsive patients. This approach can decrease the number patients needed to achieve the endpoints needed for regulatory approval and thus expedite the clinical trial and approval process for new therapeutics or treatments.

In some embodiments, any of the methods and diagnostics disclosed herein can be used in conjunction with a gene therapy, small molecule, chemotherapy, immunotherapy, surgery, radiosurgery, proton therapy, radiation therapy, photodynamic therapy, targeted therapy, or any combination thereof.

In some embodiments, methods and diagnostics disclosed herein are used with a chemotherapy, including ethothrexate, everolimus, alectinib, pemetrexed disodium, brigatinib, atezolizumab, bevacizumab, carboplatin, ceritinib, crizotinib, ramucirumab, dabrafenib, docetaxel, erlotinib hydrochloride, methotrexate, afatinib dimaleate, gemcitabine hydrochloride, gemcitabine hydrochloride, gefitinib, trametinib, methotrexate, mechlorethamine hydrochloride, vinorelbine tartrate, necitumumab, nivolumab, osimertinib, paclitaxel, carboplatin, pembrolizumab, pemetrexed disodium, necitumumab, ramucirumab, dabrafenib, osimertinib, erlotinib hydrochloride, paclitaxel, docetaxel, atezolizumab, trametinib, vinorelbine tartrate, crizotinib, ceritinib, carboplatin-taxol, gemcitabine-cisplatin, doxorubicin hydrochloride, etoposide, topotecan hydrochloride, mechlorethamine hydrochloride, topotecan hydrochloride, or any combination thereof. In some embodiments, a chemotherapy is administered to a patient who tested positive for lung cancer using the cellular response assay described herein. In some embodiments, a chemotherapy is administered in combination with another therapy, or as a combination therapy.

Several aspects are described above with reference to examples of applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein. The section headings used herein are for organizational purposes only and are not to be construed as limiting the subject matter described.

### EXAMPLES

The following examples are included to further describe some aspects of the present disclosure, and should not be used to limit the scope of the invention.

### Example 1

### Developing an Indicator Cell Assay to Distinguish Lung Cancer from Benign Nodules

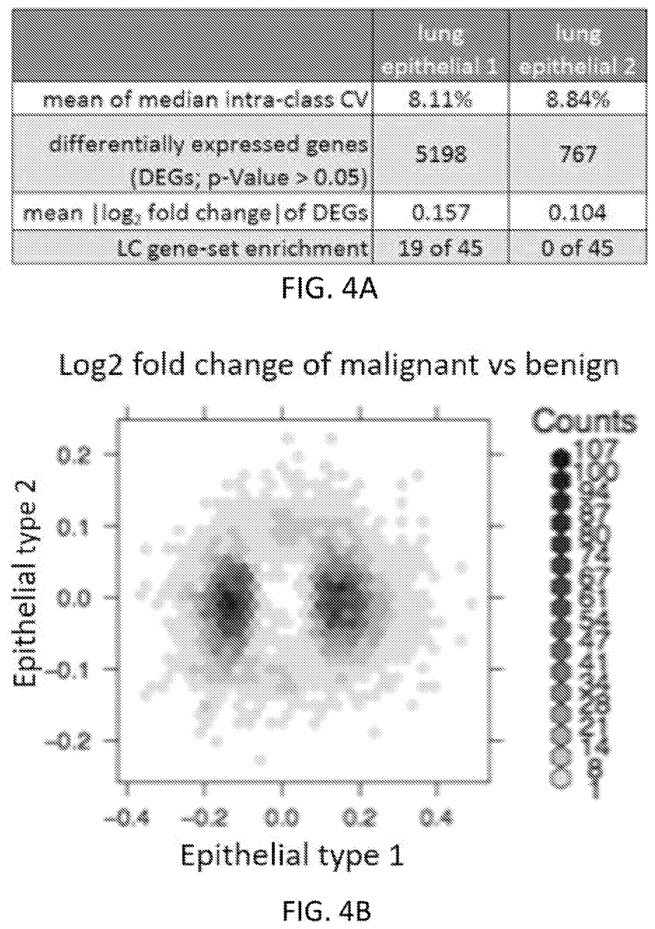

This example shows the use of blood-based indicator cell assays to distinguish lung cancer from benign nodules. As a first step, three different indicator cells were compared to identify the indicator cell population(s) with the best performance. Samples from subjects with benign or malignant lung nodules (n=6) were analyzed with the iCAP with different indicator cell types. As shown in FIG. 4A, lung epithelial cells type 1 (16HBE cells) were the best-performing cells with significantly lower intra-class CV and p-values for differentially expressed genes than other cell types. 16HBEs are useful as the indicator cells for the iCAP-lung cancer system.

To develop an iCAP with 16HBE indicator cells, 2×10⁶ 16HBE cells were thawed and plated into a T75 flask in complete growth medium (RPMI+10% FBS). Cells were trypsinized and counted and plated at 30,000 cells/cm 2 in a 12-well plate (Eppendorf) in complete medium. Cells were washed once with medium without FBS and incubated with 950 μl RPMI medium+50 μl patient serum for 24 h. Total RNA was isolated using RNeasy Mini Kit (Qiagen) and analyzed by RNAseq. All serum samples used were from patients with IPNs between 4 mm and 25 mm in size, identified by CT, which after resection or monitoring were found to be either benign or malignant (N=57 or 58 per class, respectively). Serum samples were obtained from the same source population, and matched for clinical risk factors including age, gender and smoking history. All cases were NSCL (except for one SCLC), and all controls were ascertained benign nodules by pathology or by absence of growth on chest CT after 2 years of follow up. iCAP experiments were performed in 7 batches and RNAseq was performed in 2 library preparation batches on a HiSeq4000™ (Illumina). Sequences were aligned to reference genome hg38 using STAR software, a gene count table was calculated using the R:featureCounts package, and ERCC spike-ins and genes with mean absolute counts <10 were removed. Data from the first two of 7 iCAP batches, consisting of 6 benign and 6 malignant samples, were used to determine a differential response pattern comprising a set of key response pattern features to classify the 103 lung cancer samples in the remaining batches. DESeq2 was used to identify differentially expressed genes (DEGs), based on the filtered raw count data. A total of 239 differentially expressed genes were identified at 5% FDR (Benjamini-Hochberg). To develop classifiers for use in the determination of a risk for lung cancer in a subject using key response pattern features, all RNAseq count data were then normalized using the DESeq2 r log transformation, and a series of random forest classifiers (R: randomForest package) were parameterized on normalized data from only the 12 initial samples, using increasing numbers of differentially expressed genes in rank order of FDR as features (5, 10, 20, 25, 50, 75, 100).

The remaining 103 samples were split into initial (73 sample) and final (30 sample) independent validation sets. The random forest classifiers were used to predict the initial 73 samples. All models parameterized on sets of key response pattern features comprising 20 or more DEGs made significant predictions with AUCs between 0.64-0.67. (Significance for these and other ROCs discussed below is defined as the entire 95% confidence interval of the ROC curve lying above 0.5.)

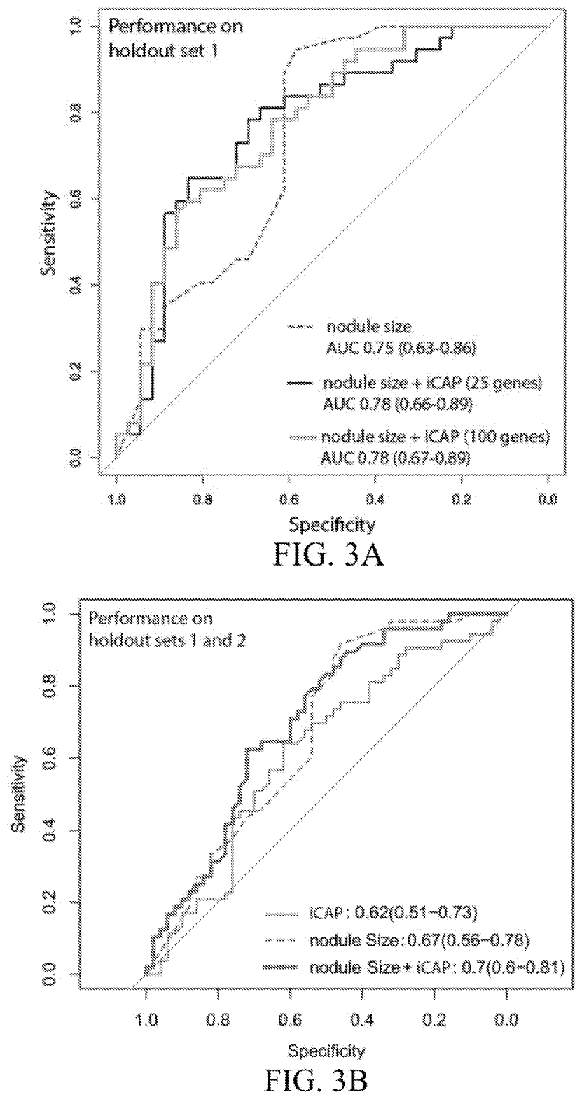

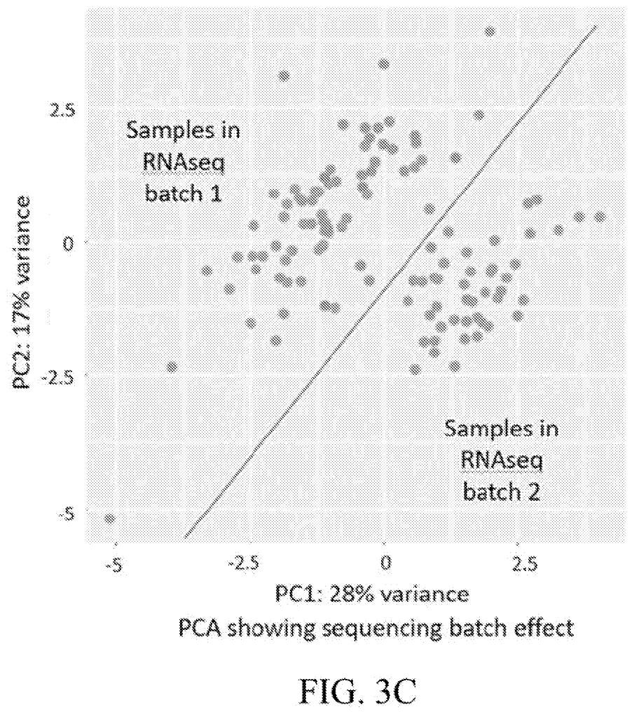

Next, the effect of adding nodule size to the set of key response pattern features on the performance of the classifiers was evaluated. To do this, a predictor score was calculated as the sum of the model prediction (0-1) and nodule size (0-30 mm) linearly scaled to a 0-1 scale. Adding nodule size to the set of features used for applying the classifier improved performance of the classifiers to 0.76-0.78 (vs 0.75 for nodule size alone) for classifiers comprised of 20, 25, 50, 75, or 100 DEGs (performances of classifiers with either 25 or 100 DEGs are shown in FIG. 3A (nodule size shown in dashed line; nodule size+25 DEGs as iCAP features shown in solid black line; nodule size+100 DEGs as iCAP features shown in solid gray line), Classifiers shown in FIG. 3A and FIG. 3B comprise all or a subset of 100 DEGs, including MACC1, CLIC4, MT1E, AKAP12, EFNB2, ITSN2, P4HA1, PDK1, STC1, IGFL1, SERPINB5, B4GALT4, KLF7, DYSF, IRF6, TPM4, F3, SESTD1, BMP6, C1orf74, ERO1A, DUS1L, ERRFI1, PLOD2, DKK1, NID2, KDM6A, EDN1, TNFRSF10D, OSMR, TFRC, RASSF3, HLA.V, MARCKS, EMP1, GAS2L1, CDCP1, DNAJC3, SOX4, GOLM1, SERINC5, LDHA, SPOCD1, PSTPIP2, PARD6B, PPP1R3B, HK2, TMEM45A, BTG1, PANX1, MYO5B, ANKRD33B, CALD1, SNX9, MORF4L2, GDNF, TRIM58, HN1L, BCAT1, PDE8A, EGLN1, KRTAP2.3, SLC9A2, JUN, ITGA3, RAP2B, SH3KBP1, PGK1, INSIG2, CRCT1, TACSTD2, ALCAM, TOR1AIP2, NMB, TPBG, OCLN, TARSL2, SAMD4A, EEFSEC, ABCC4, ITGAV, NPEPPS, RALA, AC006262.5, LGALSL, HCAR2, SLCO2A1, FHOD1, RABEP2, SLC25A37, DLG1, VEGFA, CDH1, IGFBP3, BRAT1, FAM174B, PRDM1, STS, USP53, PEAR1, (+/−nodule size) made statistically significant predictions on the holdout sets. The 25 gene model was chosen for further testing. The false discovery rate (FDR) for this model (adjusted based on the total number of gene-size combinations tested) was 0.048. First, the model was used to make blind predictions on the complete validation set (FIG. 3B, 25 DEG iCAP shown in solid thin gray line; nodule size classifier shown in dashed line; 25 DEG+nodule size classifier, having highest confidence interval, shown in thick black line). Next, with all sample classes unblinded, principle component analysis was performed, which revealed a significant sequencing batch effect in the data (FIG. 3C). The points on the graph in FIG. 3C indicate individual iCAP experimental batches for the representative examples of collected data. The diagonal line of FIG. 3C is added to illustrates the segregation of samples from different RNAseq library prep batches in the data. To mitigate this issue, the performance of the 25 DEG classifier, which was trained based on a set of 25 key response pattern features selected from the total number of differentially expressed features, was reassessed using only the 59 test samples processed in the same RNAseq library prep batch with the training set. The test showed significant performance that improved from 0.65 to 0.74 with inclusion of nodule size data (FIG. 7). Receiver operator characteristic (ROC) curves shown in FIG. 7 illustrate performances of three lung cancer classifiers (iCAP (gray line), nodule size (dashed line), and iCAP+nodule size (thick black line)) when tested with only the independent samples from the same RNAseq library prep batch as the test set (n=59). This correction yielded a ROC curve AUC of 0.74 (with nodule size data included), and a cutoff approaching clinical utility with 92% sensitivity and 38% specificity. The 25 differentially expressed genes comprising the set of key response pattern features used for analysis (e.g., used for training and applying the classifier) were CLIC4, MACC1, MT1E, ITSN2, AKAP12, EFNB2, P4HA1, PDK1, STC1, IGFL1, B4GALT4, SERPINB5, KLF7, DYSF, IRF6, TPM4, F3, SESTD1, BMP6, C1orf74, ERO1A, DUS1L, ERRFI1, PLOD2, and DKK1). These data demonstrate the iCAP has significant performance distinguishing patients with LC from those with benign nodules and that the iCAP data are complementary to nodule size data for disease classification.

In some embodiments, iCAP lung cancer is developed to distinguish patients with benign nodules from those with non-small cell lung cancer, which represents about 85% of all lung cancer diagnoses. The assay was tested with two hold-out test sets yielding a ROC curve AUC of 0.74, and a cutoff approaching clinical utility with 92% sensitivity and 38% specificity.

This example shows significant performance with a very small training set. To further improve iCAP-lung cancer configuration and increase performance in blind validation, classifier training can be repeated with an increased number of samples in the training set.

### Example 2

### Designing iCAP Configurations

This example shows the optimization of parameters of the iCAP system. For each cell type, serum concentration and serum exposure time are co-optimized in the iCAP by exploring combinations of these parameters. Three concentration levels (2.5%, 5%, and 10%) and two incubation times (6 h, and 18 h) are explored, resulting in a total of 6 experimental conditions.

For each cell type and each condition, aliquots of the same 10 case and 10 control samples are used. Each assay plate includes one assay of a reference serum sample, one of several aliquots from the same healthy donor used for normalization, quality control analysis and, if necessary, integrated into other computational analyses.

Each of the parameter sets is evaluated by evaluating strength of case versus control differential expression including maximizing the number of significantly differentially expressed genes (p-value <0.05), the magnitude of differential expression, and the enrichment of disease related processes, and minimizing within class CV to determine the set of key response pattern features to be used in classifier training and use (FIG. 4A and FIG. 4B). Significance of improvements are determined by Wilcoxon signed rank test with multiple hypothesis correction, and resampling/bootstrap-type testing when necessary.

Optimization and validation of lung cancer iCAP systems and methods can involve optimizing the experimental, technical and computational parameters of the assay to improve cellular readout; training and testing an improved lung cancer classifier using the optimal parameter sets (e.g., key response pattern feature sets) established in preliminary studies; and validating the assay with blind independent samples from at least two independent sources. Such optimization can improve clinical utility and superior performance compared to other assays, corresponding to ≤5% FNR and ≤40% FPR with >90% NPV, which provides an example of a cost-effective and high-throughput clinical iCAP to serve the clinical community.

In another approach, iCAP configurations were tested by repeated iCAP analysis of 4 technical replicates of each benign and malignant serum pool across configurations and comparison of the number of significantly differentially expressed genes for each configuration (FDR<0.1). For each class, the serum pools were comprised of serum from 8 subjects selected based on their iCAP RNAseq data to have key response pattern feature values that were representative of the training set samples in Example 1. Differential expression was measured by either RNAseq using HiSeq4000 ™ (Illumina), or by analysis of 74 genes of the key response pattern feature set described in Example 1 using nCounter® technology (Nanostring Technologies). Configurations tested were serum concentrations (1%, 5%, 10%, and 20%), serum incubation times (6 hours, 24 hours), 3 different 16HBE expansion batches, and 4 cell types (16HBE, A549, MRC5, and Nuli-1). Optimal iCAP configurations were found to be 16HBE cells, 5% plasma, and 24 hours incubation. Assay output showed stability across three expansion batches of indicator cells.

In this example, various cell types were tested as indicator cells. All cell types tested were homogeneous populations of cells from cultured cell lines. As far as practically feasible, all cells were of the same type and were genetically identical. If a heterogeneous cell population were used, even if multiple cell types were plated at the same ratio in each well, it would be useful to estimate and correct for variations in ratios of cell types over the course of the assay, as those factors may vary, e.g., due to variability in growth rates for each cell line, and variability of local environment of each cell. This variability would add to the existing variability in the assay readout due to environmental and biological diversity of the subjects, so minimization of these effects would be advantageous to maintaining high levels of detection of the disease-specific response and development of a classifier with significant performance or clinical utility.

### Example 3

### Validation of iCAP Classifiers

This example describes a means of validating iCAP classifiers. To avoid biases in the data, at each analysis stage, the fraction of samples from each source is the same for each class. For an iCAP assay, values for the set of key response pattern features are measured. Optionally, values for the set of key response pattern features along with the global response pattern pertaining to gene expression is measured.

A final classifier is established when the classifier has been rigorously tested for blind predictive accuracy with 165 independent samples from two or more independent sources using the set of key response pattern features.

Global analysis of gene expression using the entire set of response pattern features pertaining to gene expression (e.g., rather than only the key response pattern features) allows one to retrain the classifier with all data together and test accuracy by 10-fold cross validation. Such data is not necessarily used to change the final classifier configuration but can be used to measure classifier robustness and accuracy trajectory.

In some aspects, the classifier performance is improved by increasing the size of training set (e.g., the number of samples used for classifier training) to refine the selection of key response pattern features and to increase the accuracy of the classifier. In some cases, a new classifier is tested against a new, independent sample set. In addition, non-iCAP features can be incorporated into the set of features on which the classifier is trained and/or on which the classifier is applied to evaluate a response pattern, including but not limited to, clinical assessments such as age, nodule size, and smoking history into a classifier can improve accuracy.

### Example 4

### Method of Treating Lung Cancer

This example describes an improved method of treating lung cancer. Patients with a risk for lung cancer are screened using the cellular response assay or the cellular response assay in combination with a CT scan. One or more samples from a patient, such as serum samples, are analyzed using a cellular response assay for lung cancer classification. The assay is developed by exposing indicator cells to validated samples that are positive controls, such as samples from patients with lung cancer positive for EGFR or ALK mutations, and negative controls, such as samples from patients with lung cancer negative for EGFR or ALK, or from subjects without lung cancer. Assay readouts are used to identify a differential response pattern and/or to determine one or more values of a differential response pattern, such as quantitative, semi-quantitative, or qualitative changes in levels of iCAP biomarkers between affected and unaffected samples. A set of response pattern features are selected from the differential response pattern (e.g., key response pattern features) that, when measured and compared to assay readouts using positive and negative control samples, are used to accurately predict the disease status of the source of control samples by cross-validation and by validation with a holdout set. For screening, samples from a patient, such as serum samples, are incubated with cultured lung epithelial indicator cells. After normalization, the response pattern features measured from indicator cells contacted with the patient sample(s) are compared to response pattern features (e.g., key response pattern features) of indicator cells contacted with samples from positive controls and/or negative controls to predict a physiological state (e.g., disease class) of the subject. For example, a strong similarity between the measured response pattern features of an indicator cell population treated with a sample from a subject compared to the response pattern feature values measured in an indicator cell population contacted with a sample from a control subject positive for an EGFR mutation and/or an ALK mutation indicates that the patient has lung cancer (or a high risk thereof) that is related to the corresponding mutation with a calculated level of confidence based on the similarity. When the iCAP system returns results indicating that the patient has or is at high risk of having lung cancer related to an EGFR or ALK mutation, the patient is administered a therapy that is known to be most efficacious in patients with mutations in EGFR or ALK or is approved for treating patients with such mutations. After commencement of a treatment plan, patients are monitored periodically with iCAP cellular response assays to evaluate the patient's health status and efficacy of the treatment.

### Example 5

### Validating and Testing Robustness of iCAP Response Pattern Features with Independent Samples

This example shows an evaluation of independent samples using the iCAP system. To validate the iCAP for lung cancer detection, a group of differentially expressed genes were selected from the study described in Example 1 and tested in a second iCAP study with 10 new subjects from a different collection site. To test robustness of the genes, the assay was performed both under standard conditions (in three technical replicates across three iCAP batches), and with sequential changes to three experimental parameters including handling of the sample and the cells (95 assays total). First, a group of genes were selected for validation; from the data of Example 1, 182 genes were identified that were significantly differentially expressed in the iCAP between subjects with malignant nodules and subjects with no known cancer or nodules (e.g., reference samples) with FDR<0.2. From these genes, 77 were selected that also showed differential expression in the iCAP between subjects with benign and malignant nodules (p-value <0.1 for at least one of the 6 experimental batches in Example 1). Next, iCAP assays were performed with 6 new case samples from patients with malignant nodules from a different collection site than that used in Example 1, and 4 different age-matched samples with no known cancer. Expression of the 77 genes were measured by PCR-based analysis with a Biomark HD system (Fluidigm). Of the 95 samples tested, 3 were removed as technical outliers. Data were analyzed using linear mixed modeling and principal component analysis (PCA). The linear mixed modeling approach was used to separate cancer-specific differential expression from batch-specific and parameter-specific differential expression. By this approach, 39 genes had lung cancer-specific, significant differential expression in the iCAP, including ABL2, AKT3, ARSA, C2ORF69, CALD1, CBX1, COL6A1, COQ4, DDAH1, DLG1, DUSP6, EPHB6, FAM72A, FGF1, GJA5, IL18, LEPR, LRRN4, MMP9, MT1F, MT1M, MT1X, NSRP1, PLK2, PSG5, S1PR1, SFTA1P, SLC39A10, STX3, SYNPO2, TCF25, TM4SF1, TRIM65, TSKU, TXNRD1, UBE2J1, WAC, WDR13 (FDR<0.05). PCA with gene expression data was performed to use complex relationships between genes for sample stratification. This analysis identified two case samples that had exceptionally good separation from nonaffected samples, which was most prominent in the second principal component (the PC2 space). These samples had cancer spread to lymph nodes, obstructive pneumonitis, and higher histopathologic grade compared to other samples. iCAP response pattern features contributing most to this separation were 19 genes with absolute PCA loading >0.1 in PC2 including ADRA1B, SUSD2, MT1X, GPR143, GJA5, ANPEP, DUSP6, IL18, PLK2, MTMR10, CLIP4, ADGRG1, FILIP1L, LAMA1, FGF1, EPHB6, MMP9, LRRN4, TGFB2. This PCA-derived signature of 19 genes (e.g., set of key response pattern features) was used to classify all the samples from Example 1 with significant performance (AUC of ROC was 0.59 with p-value=0.047).

### Example 6

### Using a Cost-Effective Approach to Detect Differential Gene Expression Readout from the iCAP for Lung Cancer Detection

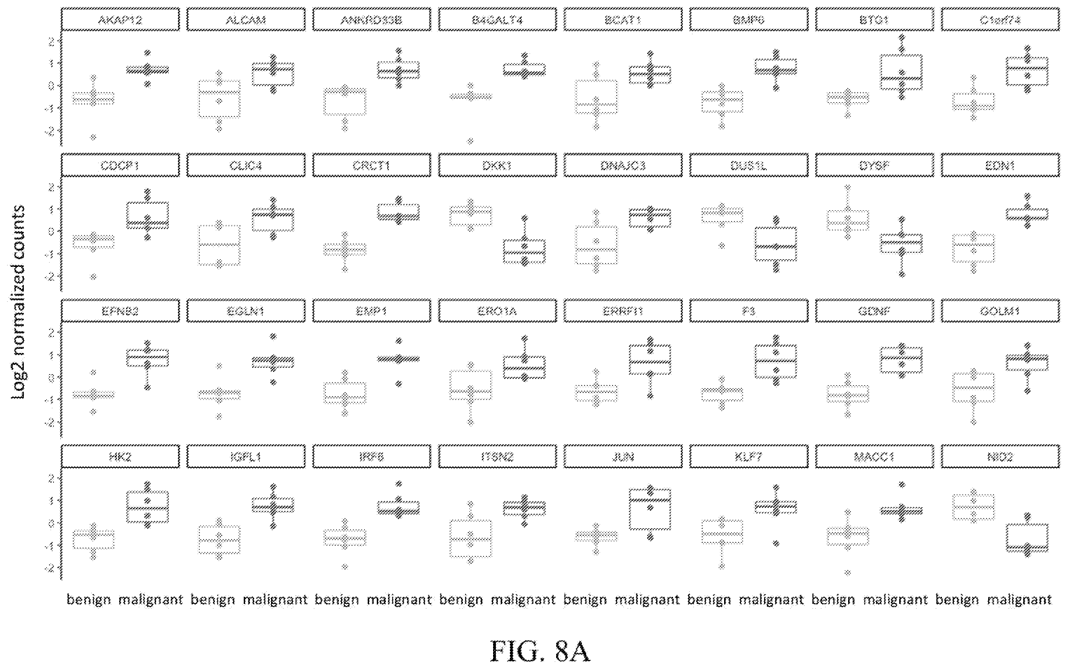

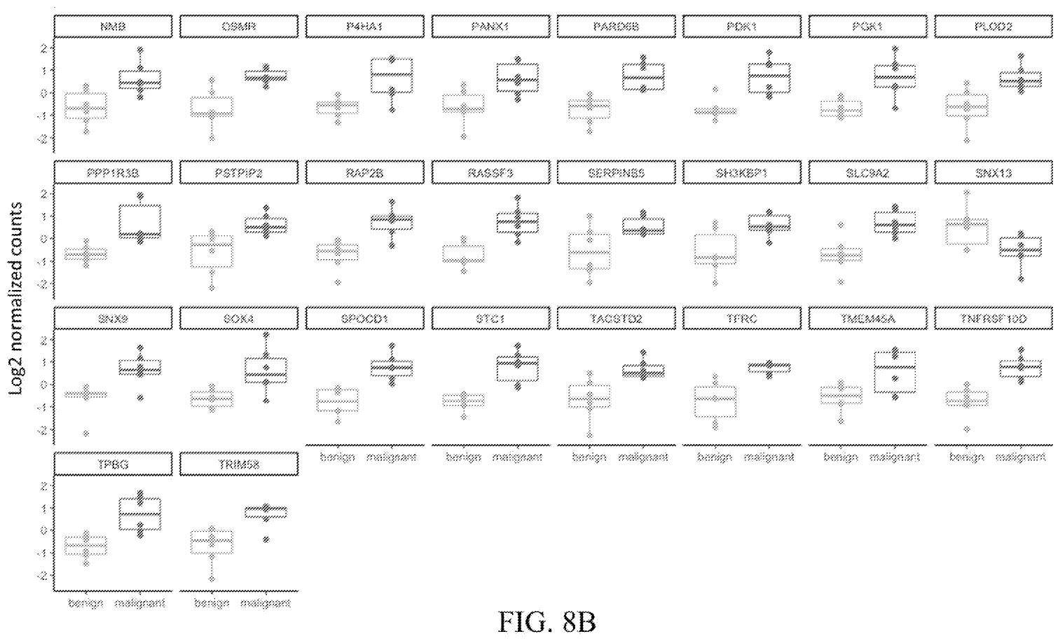

This example shows validation of differential expression observed in the training set samples detected by RNAseq in Example 1. The samples included samples from patients with malignant lung cancer and patients with benign nodules (n=6). The genes analyzed included 73 of the 100 genes in the iCAP readout used for generating the lung cancer classifier as well as 10 other genes with less robust differential expression. Differential expression was analyzed using direct nucleic acid detection with molecular barcodes technology (nCounter® technology, Nanostring Technologies). Of the 73 genes tested from the lung cancer (LC) classifier, 57 genes (78%) were significantly differentially expressed (FDR<0.1) between those with benign and malignant tumors. Of the 10 additional genes with less robust differential expression, one gene (SNX13) had differential expression (FDR<0.1) (See FIG. 8A and FIG. 8B; data shown on the left and right for each panel are from benign (B) and malignant (M) samples, respectively).

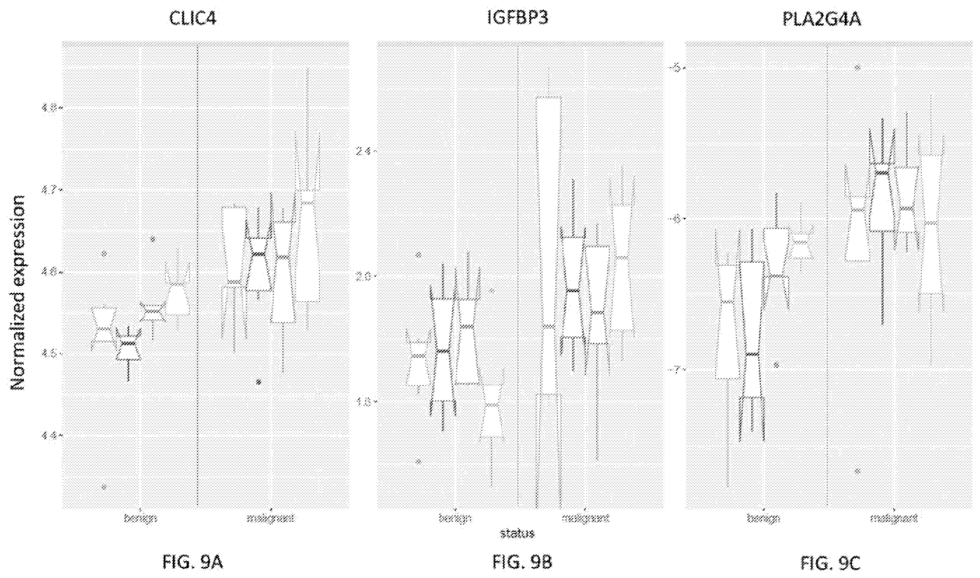

In a second experiment, differential gene expression output from the iCAP was analyzed for four experimental batches of samples from the training set and test set using a cost-effective PCR-based approach (Biomark™ HD, Fluidigm). FIGS. 9A-9C illustrate notched box plots showing gene expression levels of three iCAP biomarkers across 4 experimental batches of samples from patients with benign and malignant nodules (FDR<0.02), which were analyzed by a PCR-based approach (Biomark™ HD, Fluidigm). Samples in the training set were in the first batch (first of four box plots in each panel) and test set samples were in subsequent batches (second, third and fourth box plots in each panel). Significant differential expression between patients with benign and malignant nodules was detected across training and test set batches for three genes, CLIC4, IGFBP3, and PLA2G4A. (FDR<0.02) (FIG. 9A, FIG. 9B, and FIG. 9C, respectively).

### Example 7

### Unsupervised Hierarchical Clustering with iCAP Gene Expression Data to Separate Patients with Malignant and Benign Nodules

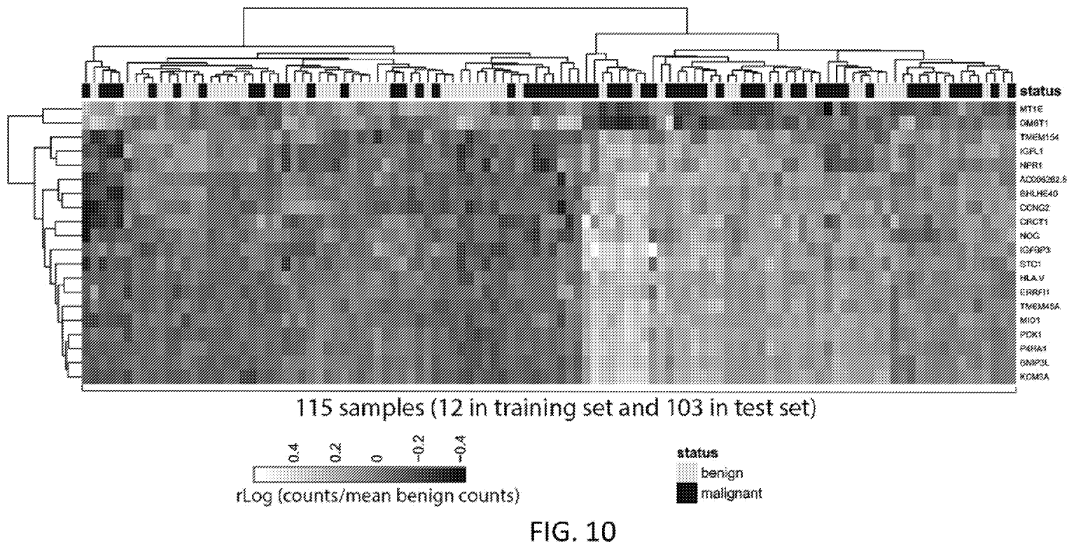

This example shows the use of an unsupervised approach to stratify patients into groups enriched for disease classes. Hierarchical clustering was used in the development of an iCAP classifier to separate samples into groups or clusters based on their iCAP gene expression profiles. FIG. 10 shows that an iCAP classifier employing hierarchical clustering successfully separated the 115 samples described in Example 1 into two groups, one enriched for patients with malignant and one enriched for patients with benign nodules, when used in the iCAP system. The dendogram at the top of FIG. 10 separates samples into two groups, one on the left and one on the right, which are enriched for benign and malignant samples, respectively. Gene expression data used to create FIG. 10 are r log-transformed counts from RNAseq data, which have been normalized to the mean expression of the benign samples of the same iCAP batch. Three benign samples were removed that appear to be outliers. Genes used to generate the clusters are the top 20 genes with the highest mean absolute deviation between malignant and benign samples of the training set only (N=6) including DMBT1, NPR1, BNIP3L, BHLHE40, MID1, CCNG2, KDM3A, TMEM154, NOG, HLA.V, CRCT1, ERRFI1, P4HA1, MT1E, PDK1, STC1, IGFL1, IGFBP3, AC006262.5, and TMEM45A. Samples clustered are the 12 samples of the training set and 103 samples of the test set used in FIG. 3. This example demonstrates the use of an unsupervised approach for clustering the data into groups to identify potential features for developing a disease classifier.

In another experiment, unsupervised hierarchical clustering was used to stratify malignant samples into groups as a way of identifying the presence of multiple iCAP signatures and multiple classes of malignant lung cancers. Clustering of malignant samples based on the expression of genes that had malignant versus benign differential expression within each iCAP experimental batch, led to the identification of a small group of malignant samples with a different iCAP profile than other samples including genes KISS1, KCP and PRSS22. With this profile and that of the other malignant samples, an ensemble classifier is trained to detect both types of malignant signatures to improve the predictive power of the iCAP. This approach for identifying malignant subgroups can also be used for improving drug discovery as some drugs have efficacy only on a subgroup of cancer patients.

### Example 8

### Lung Cancer iCAP System

This example shows the creation of a lung cancer iCAP system. Experimental and analytical covariates were computationally tested for their influence on gene expression in the RNAseq iCAP data, and several sources of variation in data were identified, including sequencing lane, experimental batch and intermittent GC (guanine-cytosine) content bias (e.g., GC-bias). Correction of these covariates increased the number of significantly differentially expressed genes across iCAP data for all 115 samples from 0 to 125 genes (FDR<0.1).

Corrections to covariates were tested to verify classifier performance. The samples were split into a 68-sample training set used for model building and 48-sample independent test set excluded from model building. The sample partition balanced the number of case and control samples of each experimental batch and sequencing lane in the training set within 20% of each other to reduce error. The test set included two complete experimental batches with no samples in the training set. A total of five 20-feature Random Forest classifiers were developed by modeling on the training set and then testing on the independent test set. Test set performance included: 1) a classifier with no GC-bias correction with accuracy of 66% (p-value 2.82E-02), 2) a classifier with conditional quantile normalization GC-bias correction with accuracy of 72.3% (p-value 2.4E-03, 3) a classifier with full quantile normalization GC-bias correction with accuracy of 70.2% (p-value 6.04E-03), 4) a classifier with GC-bias correction and nodule size included as a feature with accuracy of 76.6, (p-value 2.96E-04), and 5) one other classifier. A classifier using nodule size only had an accuracy of 66.2%. These data show that correcting for GC biases in the data and accounting for sequencing lane and experimental batch enhances detection of significant differential expression and classifier performance.

At least one of the 20-gene classifiers with GC-bias correction included 20 features selected from the following list of genes: AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318.

It was also found that classifiers with GC-bias correction comprising all 31 of the following 31 genes as features were highly accurate at differentiating between malignant and benign nodules (e.g., for determining the risk for lung cancer in a subject): AGAP1, API5, CNOT11, DNAJC5, EXOSC4, FBXO41, ITGB3BP, JRK, KCNMA1, LETMD1, LINC01588, METTL21A, MRPS15, MVB12A, MYSM1, NADK2, NIPA1, PIR, PLTP, PPIE, PPP1R12A, PRKCI, RBM17, RNF24, SNX33, TUBB, ULBP2, VGLL4, WARS, WDR45, ZNF318. In some cases, these 31 genes were used as features in a classifier of an iCAP system comprising other feature sets disclosed herein, resulting in high accuracy of the classifier.

This example illustrates that an important challenge of developing an iCAP is the need to control for technical biases in the data. Whereas the clinical version of the iCAP can use reliable, reproducible and targeted detection methods, the development of the iCAP involves the generation and analysis of large-scale data generated with high-throughput approach in some cases, which are prone to generating complex biases in the data that are not trivial to mitigate.

### Example 9

### Using a Reporter Protein to Distinguish Patients with Malignant and Benign Nodules

This example shows that a reporter protein can be used as an iCAP output to differentiate patients with benign and malignant nodules. Serum samples from 8 subjects of each benign and malignant class were selected from 115 samples based on iCAP RNAseq data and used to make benign and malignant serum pools. Next, pools of each class were analyzed in the iCAP in technical quadruplicate using the conditions described in EXAMPLE 1 using either 16HBE or Nuli-1 cells (two different bronchial epithelial cells) as indicator cells and RNAseq to measure differential gene expression.

47 and 14 genes were up and down-regulated, respectively, in malignant versus benign comparisons in both cell types (FDR<0.05) with correlation of log fold change of 0.79. Network analysis of these gene sets using STRING software showed upregulated genes were highly interconnected and enriched for genes involved in HIF1-alpha/hypoxia signaling, suggesting that transcription factor HIF1-alpha is upregulated in the malignant versus benign condition in the assay.

The benign and malignant serum pools were analyzed again in the iCAP using 16HBE indicator cells, and levels of HIF1-alpha were analyzed by Western blotting. The iCAP and Western blot experiments were performed in duplicate and HIF1-alpha expression levels were quantified using a LI-COR CLx Odyssey imaging system and compared to beta-actin as a control. HIF1-alpha was found to be 2-to-4 fold higher in the malignant versus benign replicates in both experiments.

These data demonstrate that interrogation of a protein reporter can be used as a readout of the iCAP lung cancer system to improve the accuracy of an iCAP lung cancer system in distinguishing serum from patients with benign and malignant nodules. For example, a determination of whether a subject's nodules are benign or malignant (e.g., in determining a risk of a subject for lung cancer) using an iCAP lung cancer system can be improved by including an expression level of HIF1-alpha measured in a subject's serum using Western blotting in the readout of the iCAP lung cancer system. Various quantitative, semi-quantitative, and/or qualitative methods can be used to detect or quantify protein reporters, such as HIF1-alpha. For example, protein reporters, such as HIF1-alpha, can be detected using one or more of fluorescence microscopy, cell sorting, immunoprecipitation assays, or chemiluminescence assays to enhance signal to noise and/or improve throughput of the assay.

The activation of HIF1-alpha and its target genes in the lung cancer iCAP in response to malignant versus benign samples is used in some cases to develop standard controls to monitor technical reproducibility of the iCAP readout across experimental batches. DMOG (dimethyloxalylglycine) and CoCl₂ are each known to activate HIF1-alpha. CoCl₂ and DMOG were tested at various concentrations in the iCAP up to 0.2 mM and 0.5 mM, respectively, and gene expression levels of HIF1-alpha targets were compared by amplification-free quantification of mRNA transcripts (NanoString Technologies, Inc.) to determine optimal conditions to yield differential expression in the linear range of the assay. The controls selected include performing the iCAP using standard conditions in the presence and absence of 0.25 mM DMOG and measuring HIF1-alpha targets as a readout. These controls were used to monitor assay performance across replicate batches of the iCAP and identify those with technical failure. Such controls are used in some embodiments to control assay quality in clinical deployment of the iCAP.

### Example 10

### Improving Performance and Generalizability of an iCAP for Clinical Utility Through Optimization of Feature Selection

This example shows that an aspect of developing an iCAP system with clinical utility is feature selection, including selecting the key response pattern features from the large number of potential features, and optionally combining these features with specific clinical features to optimize classifier performance. This example shows a process involving both user-directed feature selection and automated feature selection using iterative modeling and machine learning. The example shows that classifier performance and generalizability are sensitive to the feature selection method used by the user.

Feature selection is a major challenge for development of an iCAP because the response of cells to the exposure to patient biofluids can be very different from responses characterized in typical controlled laboratory studies. The high level of genetic and environmental diversity among subjects leads to a high level of variability in the assay readout between subjects of the same group or class. Therefore, even after correction for experimental and technical biases, aspects that are differential between exposure to disease and normal groups of samples tend to be variable and weak in predictive robustness. This heterogeneity includes the aspect of the response that is inferential of the disease state (e.g., which can, in many cases, include features that comprise a key response pattern for a given physiological condition). In addition, iCAP data from different sets of samples yield different disease versus normal differential expression patterns that often do not have significant overlap with each other. Thus, the best approach to identify aspects of the cellular response to patient biofluids that infer disease and generalize to new subjects is not obvious.

This example describes an analysis comparing different feature selection approaches using iterative model training and testing, which identified one approach that generated a model with superior performance and generalizability. To select iCAP gene expression features for developing disease classifiers, we used multiple approaches to identify genes that had differential expression patterns between malignant and benign conditions. We then used each feature set to train disease classifiers with and without additional automated feature reduction steps and compared the performance of the classifiers with a held-out, independent test set.

Pre-modeling data processing: For this study, serum samples of low quality (e.g., those that exhibited signs of hemolysis suggesting technical failure of sample collection) were omitted from the analysis. Serum from 141 patients (comprising a roughly equal number of samples from patients with benign and malignant nodules initially identified as IPNs on CT scans) were processed by iCAP in 8 batches, and RNA was isolated and sequenced in four batches (using the methods described in Example 1). RNAseq data were processed using R packages. RNA sequencing reads were mapped to the human genome using STAR and read counts were tabulated at the gene level using featureCounts. Counts were adjusted for GC bias using the FQN package. Genes were filtered to remove those with low counts. For modeling, the data were normalized for heteroskedasticity using VST from the DESeq2 package and for inter-iCAP batch variation using removeBatchEffect from the limma package. Three outlier samples were identified using robust principal components analysis implemented in the rrcov package and removed. The remaining samples were divided into training (65%), validation (26%), and testing (9%) groups. The training set was used for differential gene expression analysis and model training. Part of the validation set was used for pseudoblind model testing. The remaining part of the validation set and the testing set remain blinded and were not used for this analysis except for data normalization.

Feature selection: Three different methods were used to identify lists of genes that were differentially expressed between the malignant and benign classes and the gene lists were used as features for training and testing various random forest models and performances were compared. For method 1, training samples from all batches were combined and used for differential expression analysis using the DESeq2 package. For method 2, differential gene expression between malignant and benign classes was determined independently for each experimental iCAP batch using the DESeq2 package. For method 3, gene lists from method 2 were used for gene set enrichment analysis, which was performed using the fgsea package in combination with the 50 Hallmark pathway gene modules from MSigDB. Genes were ranked by absolute log fold change and filtered to those with absolute log fold change greater than 0.05 and non-adjusted p-value less than 0.05.

Feature reduction and modelling: The gene lists were used as features for generating models and the model performances were compared. For each model type, 8 different model versions were trained and tested, each with different sample filtering approaches, and for each model, 20 iterations were done with different random forest seeds. The methods for generating three top models are described below: (1) Model M4 used features selected using method 2 and was trained on samples from only one iCAP batch (batch 7) using the top 50 differentially expressed genes with an adjusted p-value less than 0.1 from another batch (batch 0). (2) Model M6 used features selected using method 2 and was trained on all training samples using the top ten differentially expressed genes from each iCAP batch. (3) Model M10 used features selected using method 3 and was trained on all training samples using nine genes with an adjusted p-value less than 0.1 in only one batch (batch 0). These genes included eight leading edge genes associated with the hypoxia module and one associated with DNA repair. Genes in models M4 and M6 were further filtered to include only 20 genes using an automated feature selection approach (e.g., selecting genes with highest variable importance in an initial round of modeling).

Models were trained using random forest implemented in the caret package. Each model, including the initial gene filtering step, was repeated 20 times initiated with different random seeds and performed with leave one out cross validation with 50 resampling iterations. Mtry values were automatically selected for each seed using the default settings except models were ordered by sensitivity rather than accuracy. Models were then tested on the partial validation set and ranked by out-of-sample AUC and specificity as well as in-sample out-of-bag AUC. M4 was trained and tested on patients with predicted forced expiry volume (FEV) greater than 50%. M6 was trained on patients that were former or current smokers at the time of serum collection and excluded samples from batch 6. M10 was trained on patients with high FEV that were current or former smokers and excluded batch 6. For M4, training samples were randomly removed with each modeling seed to balance the number of malignant and benign cases within each iCAP batch.

Results: The features of these top three models include ALPK3, ANKRD22, ANKRD37, ARMCX4, BMP6, CACNG6, CCDC66, CCNG2, CEMIP, CTF1, DEPP1, DKK1, FAXDC2, FBXL5, GPR17, HAGHL, HIF1A, IFNL2, IFNK, IGFBP3, IL1R2, KDM3A, KIRREL2, LOXL2, MT-ND4, NEDD9, P4HA1, PDK1, PDZD7, PRDM1, PRKCA, PRR22, PWP2, RASAL1, RNF223, ROR2, RSBN1, SLC2A3, SPOCD1, STC1, TFRC, TMEM45A, TRIM2. Features of model M4 include ANKRD22, RNF223, TFRC, ALPK3, CACNG6, NEDD9, STC1, HIF1A, LOXL2, PRDM1, KDM3A, GPR17, FAXDC2, DEPP1, FBXL5, TMEM45A, BMP6, P4HA1, PWP2, IL1R2. Features of model M6 include CACNG6, PRKCA, ROR2, RSBN1, PDZD7, CCDC66, ANKRD37, HAGHL, MT-ND4, BMP6, RASAL1, CEMIP, SPOCD1, PRR22, IFNL2, TRIM2, KIRREL2, CTF1, ARMCX4, IFNK. Features of model M10 include SLC2A3, STC1, PDK1, TMEM45A, KDM3A, IGFBP3, P4HA1, CCNG2, DKK1.

The top performing model was model M6, which had in-sample AUC of 0.86 and out-sample AUC of 0.78 when tested on an independent hold-out set of 27 samples. This model used gene expression levels of 20 genes to make predictions of the classes of held-out samples including CACNG6, PRKCA, ROR2, RSBN1, PDZD7, CCDC66, ANKRD37, HAGHL, MT-ND4, BMP6, RASAL1, CEMIP, SPOCD1, PRR22, IFNL2, TRIM2, KIRREL2, CTF1, ARMCX4, and IFNK. Some iCAP features identified as strongly predictive of lung cancer have not previously been shown to be associated with cancer, including gene expression levels of CACNG6, HAGHL, IFNL2, KIRREL2, CTF1, ARMCX4, and IFNK. The ROC of this model has a clinically useful cutoff with 100% sensitivity and 60% specificity, exceeding the performance of the currently available blood-based tests for lung cancer called Nodify. If this iCAP classifier were used as a rule-out test for lung cancer with this cohort of samples, 60% of patients with benign nodules would be saved from invasive follow-up procedures and 0% of patients with malignant nodules would be incorrectly classified as having a benign nodule.

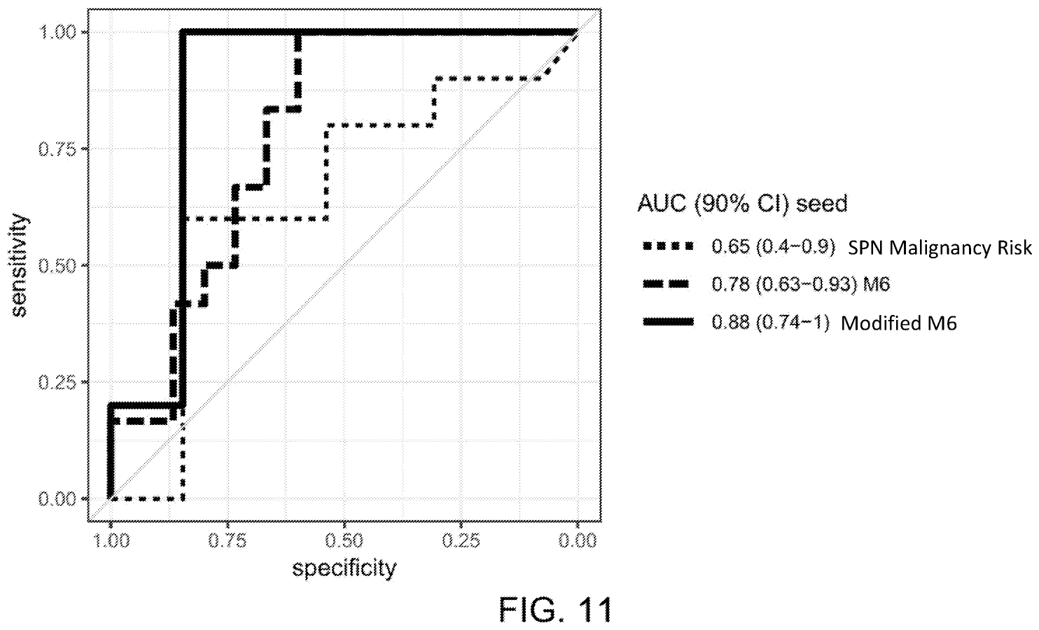

Next, we did a study, to observe the effects of including patient clinical information with iCAP data in a classifier. To do this, we made three models: 1) a model using the same features and parameters as M6 described above, 2) a Modified M6 model using the same features and parameters of M6 but also including one additional feature, which was a probability of malignancy score (based on the size, spiculation and location of the nodule, patient age, previous extrathoracic cancer diagnosis and smoking status) and 3) the SPN Clinical Malignancy Risk Score. For these analyses, the same samples and parameters used for model M6 as described above were used except, 23 samples were omitted because they did not meet criteria for diagnosis with SPN Clinical Malignancy Risk Score model. For each of the three models, 20 iterations of training/testing were performed with different seed and the seed with top performance is shown in FIG. 11. Inclusion of patient clinical data in the iCAP Modified M6 model (e.g., including probability of malignancy score), improved the performance of the model. The ROC curve for the combined Modified M6 model has AUC of 0.88 a clinically useful cutoff with 100% sensitivity and 85% specificity. If this iCAP classifier were used as a rule-out test for lung cancer with this cohort of samples, 85% of patients with benign nodules would be saved from invasive follow-up procedures and there would be no false negatives (e.g., none of the patients with malignant nodules would be incorrectly classified as having a benign nodule).

Comparison of performances of models generated for this analysis, shows that to maximize generalizability and performance of the classifier, feature selection should be based on data from as many samples as possible; however, the standard approach of merging data from all training samples together to find a differential pattern of gene expression across all available samples does not yield the best performing classifier. Instead, the best performing classifier (M6) used features selected from various different malignant versus benign differential expression patterns, each identified using various different subsets of samples. This method of differential expression analysis is not a standard approach.

All iCAP models described in this example used a random forest approach. We selected this approach because there is a high level of biological and environmental diversity of the patients within each class, including diversity of disease state between patients. This is reflected by the identification of multiple differential expression patterns from the iCAPs with different subsets of disease and normal samples. Random forest can be well suited for iCAP data because it is a learning method that makes predictions based on multiple decisions, each considering a different subset of samples and features, thus enabling the capture of diverse disease patterns, improving generalizability and performance.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-103. (canceled)

104. A method of determining a risk for lung cancer in a subject, the method comprising:
a) contacting a first indicator cell population with a sample from the subject;
b) determining a first response pattern;
c) contacting a second indicator cell population with a sample from a second subject, the second subject having a known risk for lung cancer;
d) determining a second response pattern of the second indicator cell population; and
e) determining a risk for lung cancer of the first subject based on the first response pattern and the second response pattern.

105. The method of claim 104, wherein the first indicator cell population comprises an alveolar cell, a lung epithelial cell, an immune cell, an endothelial cell, a fibroblast, or a combination thereof.

106. The method of claim 105, wherein the second indicator cell population comprises an alveolar cell, a lung epithelial cell, an immune cell, an endothelial cell, a fibroblast, or a combination thereof.

107. The method of any one of claim 104, wherein the first indicator cell population or the second indicator cell population comprises a clonal cell population derived from stem cells.

108. The method of claim 104, wherein the first indicator cell population or second cell indicator population are chosen by identifying a cell population that comprise one or more of the following characteristics when compared to other indicator cell populations:
greater number of significantly differentially expressed genes and magnitude of differential expression;
fewer median intra-class coefficient of variation (CV); or
greater significant enrichment of lung cancer-related gene sets amongst the differentially expressed genes.

109. The method of claim 104, wherein the first indicator cell population or second cell indicator population are chosen by identifying a cell population that comprises an optimized indicator cell population, wherein the optimized indicator cell population are selected by using the method comprising:
a) performing the method of claim 104, wherein the first subject and the second subject have a known risk of lung cancer; and
b) comparing the determined risk of lung cancer with the known risk of lunger cancer, wherein the optimized indicator cell population has greater correlation between the determined risk of lung cancer with the known risk of lunger cancer than other indicator cell populations.

110. The method of claim 104, further comprising determining a set of key response pattern features based on the second response pattern.

111. The method of claim 110, wherein determining the risk for lung cancer of the first subject is based on the set of key response pattern features of the second response pattern and a set of key response pattern features of the first response pattern.

112. The method of claim 104, further comprising determining a third response pattern of a third indicator cell population.

113. The method of claim 112, further comprising contacting the third indicator cell population with a sample from a third subject, the third subject having a second known risk for lung cancer.

114. The method of claim 104, further comprising determining a response pattern for each of one or more additional indicator cell populations.

115. The method of claim 111, further comprising contacting each of the one or more additional indicator cell populations with a sample from one or more additional subjects.

116. The method of claim 115, further comprising determining a differential response pattern based on two or more of the second response pattern, the third response pattern, or the response pattern for each of the one or more additional indicator cell populations.

117. A system for determining a risk of lung cancer in a first subject comprising:
a) a first indicator cell population;
b) a sample from the first subject;
c) an imaging module configured to detect a first signal from the first indicator cell population; and
d) a computer in communication with the detector, comprising a processor and a non-transitory memory on which is stored instructions that, when executed, cause the processor to: determine the risk for lung cancer in the first subject based on the first signal using a classifier stored in the non-transitory memory of the computer.

118. The system of claim 117, further comprising:
e) a second indicator cell population; and
f) a sample from a second subject having a known risk for lung cancer,
wherein the imaging module is configured to detect a second signal from the second indicator cell population; and
wherein the instructions, when executed further cause the processor to:
determine a first response pattern based on the first signal
determine a second response pattern based on the second signal,
determine a risk for lung cancer of the first subject based on the first response pattern and the second response pattern using the classifier.

119. The system of claim 118, further comprising:
g) a third indicator cell population; and
h) a sample from a third subject having a known risk for lung cancer different from the second subject,
wherein the imaging module is configured to detect a third signal from the third indicator cell population; and
wherein the instructions, when executed further cause the processor to:
determine a third response pattern based on the third signal; and
determine a risk for lung cancer of the first subject based on the first response pattern, the second response pattern, and the third response pattern using the classifier.

120. The system of claim 117, wherein the instructions, when executed, cause the processor to determine a response pattern for each of one or more additional indicator cell populations after the one or more additional indicator cell populations are contacted by a sample of one or more respective additional subjects.

121. The system of claim 120, wherein the instructions, when executed, cause the processor to determine a differential response pattern based on two or more of the second response pattern, the third response pattern, or the response pattern for each of the one or more additional indicator cell populations.

122. The system of claim 117, wherein the first indicator cell population comprises an alveolar cell, a lung epithelial cell, an immune cell, an endothelial cell, a fibroblast, or a combination thereof.

123. The system of any one of claim 118, wherein the second indicator cell population comprises an alveolar cell, a lung epithelial cell, an immune cell, an endothelial cell, a fibroblast, or a combination thereof.
